(19) United States
(12) Patent Application Publication
(10) Pub. No.: US 2026/0199678 A1
(43) Pub. Date: Jul. 16, 2026

(54) NEUROMODULATION DEVICES AND METHODS

(71) Applicant: Cala Health, Inc., San Mateo, CA (US)

(72) Inventors: Anne Brantigan, San Mateo, CA (US); Alexander R. Kent, Mountain View, CA (US); Crystal Knodel, San Mateo, CA (US); Kathryn H. Rosenbluth, San Francisco, CA (US); Gregory T. Schulte, Oakland, CA (US); Kyra Wells, Burlingame, CA (US); Belen Szentes, San Mateo, CA (US); Raghav Singhal, San Mateo, CA (US)

(21) Appl. No.: 19/121,745

(22) PCT Filed: Oct. 18, 2023
(86) PCT No.: PCT/US2023/035386

§ 371 Date: Apr. 16, 2025

Related U.S. Application Data
(60) Provisional application No. 63/380,225, filed on Oct. 19, 2022.

(51) Int. Cl. A61N 1/36; A61N 1/04

(52) U.S. Cl. CPC A61N 1/36036; A61N 1/0456; A61N 1/0476; A61N 1/36031

(57) ABSTRACT

Neuromodulation systems are provided in some aspects that are configured for placement inside the ear with a pressure applicator to bias electrodes to provide facilitate conductivity, for example, in a direction towards the ear for auricular neurostimulation. Neuromodulation systems are also provided in some aspect that can include one or more sensors that measure data from one or more biomarkers of a user's physiological state and an optional controller that uses the measured data to adjust one or more stimulation parameters.

### REFERENCE TO RELATED APPLICATIONS

This application claims the benefit of U.S. Provisional Application No. 63/380,225, filed Oct. 19, 2022, which is hereby incorporated by reference in its entirety.

### TECHNICAL FIELD

The present disclosure relates to devices, methods, and/or systems for neuromodulation (such as noninvasive nerve stimulation for example) for treatment of various conditions.

### SUMMARY

Wearable systems with compact, ergonomic form factors are needed to enhance efficacy, compliance, and comfort while using those systems. In several embodiments, a neuromodulation system is provided that comprises an earpiece and an electrical stimulation pulse generator, wherein the electrical stimulation pulse generator is configured to deliver a plurality of electrical stimulation pulses to the earpiece, which is configured for placement at least partially or fully in, on or near an ear. The earpiece, in some embodiments, comprises a body with a boot. In several embodiments, the boot comprises a housing with a material for placement on a portion of skin. In several embodiments, the boot is configured to rest on a portion of the ear (including but not limited to a concha (e.g., concha cymba and/or concha cavum), helix, scapha, anti-helix, triangular fossa, superior crus, inferior crus, helicis crus, tragus, intertragic notch, lobule, or anti-tragus of the ear, or combinations thereof), a first and second electrode protruding from the boot, a first pressure applicator comprising a first spring loaded actuating surface connected to the first electrode, and a second pressure applicator comprising a second spring loaded actuating surface connected to the second electrode. The boot may be made at least partially of silicone and designed to rest at an entrance of an ear canal. In some embodiments, the first electrode and the second electrode are configured to deliver the plurality of electrical stimulation pulses to stimulate one or more nerves in or around the ear such as an auricular nerve (such as the auricular branch of the vagus nerve, the greater auricular nerve, and/or other nerves that innervate the ear such as the ear canal, tragus, and/or auricle). In one embodiment, the first pressure applicator is configured to bias the first electrode in a first direction towards the ear by increasing a level of a first pressure at the first electrode against the ear so as to lower a first impedance between the first electrode and the ear, and the second pressure applicator is configured to bias the second electrode in a second direction towards the ear by increasing a level of a second pressure at the second electrode against the ear so as to lower a second impedance between the second electrode and the ear. In one embodiment, there is only one pressure applicator and one electrode. In some embodiments one pressure applicator is used for a single electrode or multiple electrodes. When two or more electrodes are used, they can be used to stimulate the same or different nerves. The electrical stimulation pulse generator may be separate from or integrated into the earpiece. Sensors may or may not be included in certain embodiments with pressure applicators.

In some embodiments, the neuromodulation system comprises one or more sensors that, for example, measure a biomarker. Such sensor may be on, couple to, or unattached and in communication with the earpiece or other worn device, such as on the wrist or leg. A sensor may integrated into the stimulator device, a skin patch, or a belt. One sensor may measure one biomarker or one sensor may measure two or more biomarkers. The biomarker may be indicative of a condition of a user, such as migraine, colitis, irritable bowel disease, rheumatoid arthritis, hypertension, or atrial fibrillation. If the biomarker is higher or lower than the desired level, at least one electrode is configured to deliver electrical stimulation to treat the condition (e.g., a symptom of the condition or the condition itself). In one embodiment, a sensor may detect that a heart rate is too low, high or variable and as a result neurostimulation is provided based on that feedback based on the user's control, the physician's control or automatically. The neurostimulation can then treat the biomarker (e.g., the undesired heart rate) and/or treat the underlying condition (cardiac arrythmia). In another embodiment, as sensor may detect altered electrodermal or EEG activity as biomarkers, and based on that sensed information, provided neurostimulation to treat migraine. In yet other embodiments, body temperature is used as a biomarker and based on a sensor determining that temperature is either lower or higher than the desired range, neurostimulation is provided to treat conditions (including but not limited to colitis, inflammation, arrhythmia, migraine or rheumatoid arthritis). Neurostimulation may be provided based on sensor information based on the user's control, the physician's control or automatically. A closed feedback loop may be used in some embodiments.

In some embodiments, the method provides multiple therapy pathways that are at least in part dependent on sensing of user biomarkers (e.g., heart rate, heart rate variability, heart rhythm, skin sympathetic nerve activity, electrodermal activity, body temperature as measured on the wrist, respiratory cycle, brain electrical activity, cytokine levels, physical activity, oxygen levels, etc.). In some embodiments, biomarkers can include patient demographics, prior medication usage, prior device therapy usage, and/or sleep cycles. In some embodiments, additional data such as weather or other information in the patient's local address (e.g., air temperature, humidity, pressure, elevation, etc.) can be monitored to impact stimulation and/or treatment. In one embodiment, physical activity can be measured with a motion sensor. In one embodiment, oxygen levels can be measured with a pulse oximeter (e.g., via pulse oximetry). For example, in certain embodiments, the method can provide not only an acute relief pathway but also a reduction of symptoms and/or preventative therapy pathway depending on the values of the sensed biomarkers. A preventative therapy may be one that reduces the onset, number, duration or severity of symptoms by at least 25-75% or more. As an example, through neurostimulation described herein, the severity or duration of symptoms may be significantly reduced as compared to not using such neurostimulation. For example, depending on the sensed level of the one or more biomarkers, the wearable device can determine whether the user is likely currently experiencing the condition or is instead about to experience the condition and then apply peripheral nerve stimulation via the wearable systems disclosed herein (e.g., wrist worn device, an auricular worn device, or any combination of wrist worn and auricular devices) that is effective for the therapy pathway actually being experienced at that time by the user.

In some embodiments, the wearable systems disclosed herein deliver electrical stimulation in or around the ear (for example, to the auricular branch of the vagus nerve and/or other nerves that innervate the ear) while also sensing one or more biomarkers of the user to increase efficacy of the electrical stimulation. For example, in some embodiments, the wearable system is configured as an earpiece which includes electrodes, a controller, and at least one sensor. The sensor on the earpiece can be configured to sense levels of the one or more biomarkers and provide those levels to the controller for adjusting parameters of the electrical stimulation. Stimulating the vagus nerve in this way can deliver various therapeutic benefits, including treatment of atrial fibrillation or other cardiac arrhythmias, colitis, rheumatoid arthritis, migraine, irritable bowel disease, hypertension, etc. The earpiece may be used in tandem with other neuromodulation devices (for example, those worn on the wrist) to further enhance therapeutic benefits.

In some embodiments, the wearable systems disclosed herein can determine a user's current respiratory phase and/or when a respiratory phase begins or ends. Being able to precisely determine respiratory phases can be advantageous for timing and efficacy of electrical stimulation. For example, improving timing correspondence between application of electrical stimulation when performing respiratory-gated peripheral nerve stimulation and the user's current respiratory phase increases treatment efficacy. In some embodiments, the wearable system includes a sensor which senses quantitative values relating to the respiration state of the user which alone may not be sufficient to precisely determine the user's current respiratory phase and/or when a respiratory phase begins or ends. Some embodiments of the wearable system disclosed herein includes an algorithm which takes the sensed quantitative values and then determines various user parameters (e.g., respiration threshold, sample check count, respiration slope threshold, and/or lockout length) which are indicative of whether the user is inhaling or exhaling.

Disclosed herein are various embodiments of devices, systems, and methods for delivering electrical neuromodulation (e.g., stimulation of one or more nerves) to a user. In some embodiments, the electrical stimulation is delivered to regions at or proximate an ear of the user. In some embodiments, alternatively or in addition to delivering stimulation to regions at or proximate the ear, electrical stimulation is delivered to regions at or proximate the wrist of the user. For example, in certain embodiments, an auricular device secured at least partially within an ear canal and/or a wrist worn device applies the electrical stimulation to the user. When electrical stimulation is applied to both locations, the stimulation modality parameters (e.g., frequency, phase, timing, amplitude, offsets, etc.) may be complementary so as to increase efficacy for treating the condition and/or symptoms of the condition.

In one embodiment, the electrical neuromodulation is delivered via an electrode (e.g., 1, 2, 3, 4, 5, or 6 electrodes). In several embodiments, the electrode comprises a base material and a loaded material. In one embodiment, the electrode is a dry electrode. A dry electrode may be advantageous, in some embodiments, by providing a dry skin interface between the electrode and the skin of the user without the need for adhering a hydrogel at the skin interface. The advantage of using dry electrodes is particularly acute for body-worn stimulation devices intended for long-term, repeated wear. In one embodiment, the electrode material conforms to the body, is flexible, couples well with the body, and is biocompatible. In one embodiment, a method framework for treating rheumatoid arthritis, atrial fibrillation or migraine with peripheral nerve stimulation comprises one or two therapy pathways: (a) an acute relief pathway, and/or (b) a reduction of symptoms and/or preventative therapy pathway. In one embodiment, the therapy framework and each therapy pathway may be executed by applying peripheral nerve stimulation via a wrist worn neuromodulation device, an auricular neuromodulation device, or any combination of wrist worn devices and auricular devices. In one embodiment, algorithms for detecting inspiration and expiration phases of respiration are used with an electrical neuromodulation as described herein. In some embodiments, dry electrodes, unlike wet electrodes, provide a dry skin interface between the electrode and the skin of the user without the need for adhering a hydrogel at the skin interface. The advantage of using dry electrodes is particularly acute for body-worn stimulation devices intended for long-term, repeated wear according to some embodiments. In some embodiments, the electrode material is flexible which allows the skin interface of the dry electrode to maintain contact with a surface of the body (e.g., arm, wrist, leg, ear, etc.) when repeatedly worn. The electrode material is further not harmful to living tissue (e.g., biocompatible).

In various embodiments, inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases (such as neuroinflammation) are treated. Cardiac conditions (such as atrial fibrillation, hypertension, and stroke) are treated in various embodiments. Epilepsy and other seizure disorders are treated in one embodiment. Headaches, such as migraine, are treated in other embodiments. Inflammatory skin conditions and immune dysfunction may also be treated in certain embodiments. In several embodiments, cytokine signaling proteins that help control inflammation are affected (e.g., decreased or balanced) by stimulation.

In various embodiments, such neuromodulation may be beneficial to treat inflammation (such as neuroinflammation), movement disorders, cardiac disorders, pain, psychiatric disorders, and other conditions. Some of the disclosed devices, systems, and methods can advantageously stimulate the vagus (aka vagal) nerve of a user while accommodating wide variation in ear anatomy and/or other characteristics across different users. In some embodiments, placement of an earpiece and/or stimulation is provided at, near or within the concha cymba and/or one or more of the following locations in or around the ear, such the helix, scapha, anti-helix, triangular fossa, superior crus, inferior crus, helicis crus, tragus, intertragic notch, lobule, anti-tragus, and concha cavum, or combinations thereof. In some embodiments, devices are provided that provide stimulation (e.g., vagal nerve stimulation for example, via the concha (e.g., concha cymba and/or concha cavum) and which can accommodate high variations in ear anatomy from person to person. Gradual increases or other alternations in burst pattern stimulation are provided in several embodiments and may, for example, help with patient comfort, compliance, habituation, and/or efficacy. The terms “vagal” and “vagus” can be used interchangeably herein.

In several embodiments, neuromodulation, such as neurostimulation, as used herein is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, gastrointestinal (“GI”) issues, nausea, confusion, dyskinesia, altered appetite, etc. In various embodiments, neuromodulation, such as neurostimulation, is used together with (e.g., synergistically with) pharmaceutical agents for treatment of epilepsy, depression, anxiety, inflammatory conditions such as inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory conditions (such as neuroinflammation and inflammatory skin conditions) are treated in several embodiments. Neuromodulation, such as neurostimulation, is used together with (e.g., synergistically with) pharmaceutical agents for treatment of cardiac conditions (such as atrial fibrillation, hypertension, and stroke) are treated in various embodiments. Neuromodulation, such as neurostimulation, is used together with (e.g., synergistically with) pharmaceutical agents for treatment of headaches, such as migraine, are treated in other embodiments. Neuromodulation may be delivered at the same time of day as the drugs are used. Alternatively, neuromodulation may be delivered at different times of day than the drugs are used, or neuromodulation may be delivered on different days than the drugs are used. Drugs may be used in lower doses, for shorter times, and/or with fewer side effects when used with the neurostimulation embodiments described herein. For example, a drug time course or dose may be reduced by 10-90% (e.g., 10-30%, 30-60%, 60-90% and overlapping ranges therein) when used with neurostimulation as described herein. Patient may also be able to tolerate a drug or other therapy for a longer time when used with neurostimulation as described herein because the side effects are reduced.

In some embodiments, the devices described herein (such as an auricular, leg or wrist device) employs a gradual stimulation burst pattern. In some embodiments, the auricular device employs the gradual stimulation burst pattern with respiratory gating. In some embodiments, the system may deliver the burst of stimulation pulses at a specific frequency (e.g., theta bursts in, for example, the 4-8 Hz range) that is dependent on the respiration cycle. In some embodiments, the auricular device employs the gradual stimulation burst pattern without respiratory gating. In some embodiments, the system may deliver the burst of stimulation pulses at a specific frequency (e.g., theta bursts in, for example, the 4-8 Hz range) that is not dependent on the respiration cycle. For example, in certain embodiments, the system may apply a gradual increase in stimulation intensity at the beginning of each burst of stimulation pulses. The gradual stimulation burst pattern can enhance patient comfort for certain patients (e.g., where gated or other stimulation may be uncomfortable and surprising for a user). For example, when a pulse applied to an ear turns on at full power, there may be no ramping period to mask the intensity of stimulation sensations to the ear. The gradual stimulation burst pattern can comprise a lower stimulation intensity that gradually increases to a higher intensity over the burst to provide a measure of ramping to the stimulation intensity.

In one embodiment, a method of determining the respiratory phase of a user includes the use of a sensor for detecting and measuring respiration. This sensor produces some quantitative measure relating to the respiration state of the user. In some embodiments, the respiration state is measured with mechanical, electrical, impedance, acoustic (e.g., microphone), ultrasonic, infrared, or video based measures. A controller then receives this value from the sensor and applies an algorithm that uses various parameters to determine whether a person is inhaling or exhaling. In one embodiment, one such parameter that the algorithm uses is a respiration threshold. This threshold value is the minimum difference in amplitude between two sample values obtained from the sensor. In one embodiment, a second potential type of parameter may be a sample check count. This sample check count is the minimum number of samples in a row required to be checked in order to consider whether the user has switched from one respiratory phase to another (e.g., inhalation to exhalation). In one embodiment, a third potential type of parameter might be the respiration slope threshold. The respiration slope threshold is the minimum slope value to assign a change from one respiratory phase to another (e.g., inhalation to exhalation). In one embodiment, a fourth potential parameter could be a lockout length. The lockout length is a minimum amount of time in which the algorithm is paused. The algorithm might also include a genetic evolutionary algorithm, a machine learning algorithm, or some other algorithm based in artificial intelligence. In these cases, the algorithms might rely on as few as zero parameters to determine the respiratory phase of the user.

In some embodiments, the gradual burst pattern is generated by one or more hardware processors of the system (such as an auricular, leg or wrist device). In some embodiments, the system can sense an increase or decrease in one or more parameters sensed by one or more sensors selected from the group consisting of a photoplethysmography sensor (PPG), a galvanic skin sensor (GSR), an inertial measurement unit sensor (IMU), a temperature sensor (e.g., for body/skin temperature or ambient temperature), a respiratory sensor, and an electroencephalography sensor (EEG), and combinations thereof. Based on the sensed increase or decrease, the system can tune the burst of stimulation pulses to the one or more parameters. In some embodiments, the system can tune or change one, two, or more stimulation modality parameters (e.g., frequency, phase, timing, amplitude, offsets, etc.) of the burst of stimulation pulses accordingly. Such tuning can be implemented for any of the described modalities herein (e.g., epilepsy, depression, migraine, vagus nerve stimulation (VNS), etc.).

In some embodiments, a first portion of the auricular device secures at least partially within an ear canal of a user while a second portion coupled with the first portion is placed adjacent (e.g., next to, within or in contact with) the concha cymba of an ear of the user. In several embodiments, contact with a helix, scapha, anti-helix, triangular fossa, superior crus, inferior crus, helicis crus, tragus, intertragic notch, lobule, anti-tragus, concha cavum, and/or concha cymba of an ear or combinations thereof is performed. In some embodiments, the auricular device comprises a third portion that has a stem and a boss. In some embodiments, the first portion comprises an ear canal element with the stem being slidably connected to the boss and rotatably connected to the ear canal element via the boss. In several embodiments, the gradual stimulation burst pattern is applied by a nerve effector (such as one, two, four or six electrodes) of the second portion and gently ramps up in intensity. The gradual stimulation burst pattern can ramp up in different ways. For example, in some embodiments, the lower stimulation intensity gradually increases to the higher intensity over a plurality of pulses of the gradual stimulation burst pattern. In some embodiments, the lower stimulation intensity gradually increases to the higher intensity over an initial pulse of the gradual stimulation burst pattern. In some embodiments, the stimulation may ramp up by 0.05-5.0 mA (e.g., 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0 mA, and overlapping ranges therein) over a period of 0.1-30 or more seconds (e.g., 0.1-0.5, 0.5-2, 2-5, 5-10, 10-15, 15-30 seconds, and overlapping ranges therein) or the ramp up may increase by about 10-50%, 50-100% or double or triple with each increment. The differential between the increments may be constant or different. For example, the gradual increase or ramp up may be 0.1 mA to 0.2 mA to 0.3 mA to 0.4 mA to 0.8 mA to 1.0 A to 1.6 A to 3.2 A (and higher if needed). Alternatively, the gradual increase or ramp up start at 0.5 mA and increase constantly by 0.2 mA to reach a setpoint such as 2.5 A. A ramp up may occur each time the stimulation is turned on initially or may occur during a treatment session when a user increases stimulation. A user may adjust the ramp up or it may be automated by the system. Ramp downs are included in some embodiments. In some embodiments, the gradual increase or ramping may also be used for non-burst stimulation (such as tonic stimulation).

In some embodiments, a method of neuromodulation or use of the systems described herein are provided to treat a condition and/or its symptoms. Conditions include but are not limited to rheumatoid arthritis, atrial fibrillation and migraine. The method can comprise positioning a first electrode against the patient's skin proximate a first peripheral nerve, positioning a second electrode against the patient's skin proximate the first peripheral nerve or a second peripheral nerve, and sensing a level of a biomarker related to the condition. If the sensed level is indicative of the user experiencing the condition, delivering, via the first and second electrodes, a first electrical stimulation to provide acute relief therapy. If the sensed level is indicative of the user being at an onset of the condition delivering, via the first and second electrodes, a second electrical stimulation to provide a preventative therapy. A preventative therapy may be one that reduces the onset, number, duration or severity of future symptoms or the condition itself by at least 25-75% or more. (e.g., 25, 40, 50, 70, 90%, etc.). A value of a stimulation modality parameter of the first electrical stimulation is different than a value of the stimulation modality parameter of the second electrical stimulation. The stimulation may be provided to nerves in and around the ear, arm (e.g., wrist) and leg (e.g., thigh, knee and ankle). In one embodiment, the biomarker is heart rate, and if the sensed level is indicative of the user experiencing a condition, delivering electrical stimulation via the first and/or second electrodes to provide relief therapy. In one embodiment, the biomarker is heart rate variability, and if the sensed level is indicative of the user being at an onset of experiencing or having rheumatoid arthritis, atrial fibrillation or migraine, delivering electrical stimulation via the first and/or second electrodes to provide therapy (such as preventative therapy to decrease the onset, number, duration or severity of symptoms by at least 25-75% or more (e.g., 25, 40, 50, 70, 90%, etc.). At least one nerve of the one or more targeted first nerves or of the one or more targeted second nerves is not common to both the one or more targeted first nerves and the one or more targeted second nerves. Additional sensors and/or electrodes may also be used.

In some embodiments, the stimulation modality parameter can be amplitude, and the value of the stimulation modality parameter of the first electrical stimulation can be lower than the value of the stimulation modality parameter of the second electrical stimulation. The stimulation modality parameter can be frequency. The stimulation modality parameter can be pulse width. The first electrode and the second electrode can be disposed on a neuromodulation device. The neuromodulation device can be configured as a wrist worn device. The first electrode and the second electrode can be disposed on a neuromodulation device. The neuromodulation device can be configured to be worn in or proximate the ear. One of the first electrode (or first set of electrodes) and the second electrode (or second set of electrodes) can be configured to be disposed on a wrist of the user and the other one of the first electrode and the second electrode can be configured to be worn in or proximate another location (such as the ear, other wrist or leg).

In some embodiments, the sensing of the level of the biomarker can occur during a detection phase, and the acute relief therapy and the preventative therapy can occur during a therapy delivery phase and after the detection phase. The sensing of the level of the biomarker can be performed by one or more sensors, and the first electrode, the second (or third, fourth, or additional) electrode, and the one or more sensors can be integrated into a neuromodulation device, the detection phase can be performed by patient self-reporting of symptoms.

In some embodiments, the therapy delivery phase can be commenced by directing a user to initiate delivering the first electrical stimulation to provide the acute relief therapy or delivering the second electrical stimulation to provide the preventative therapy. The therapy delivery phase can be commenced automatically, in one embodiment, after the sensed level is indicative of the user having for example rheumatoid arthritis, atrial fibrillation or migraine, or the user being at an onset of having rheumatoid arthritis, atrial fibrillation or migraine. In one embodiment, preventative therapy decreases the onset, number, duration or severity of future symptoms by at least 25-75% or more (e.g., 25, 40, 50, 70, 90%, etc.).

In some embodiments, the biomarker measured can be at least one of heart rate, heart rate variability, heart rhythm, skin sympathetic nerve activity, electrodermal activity, body temperature as measured on the wrist or ear, the respiratory cycle, brain electrical activity, and/or cytokine levels. The biomarkers may be measured by sensors located on the body (such as the ear, forehead, scalp or other parts of the head, the wrist or other parts of the arm, the ankle or other parts of the leg, the chest, etc.). A machine learning algorithm can be used to assess when to commence the therapy delivery phase depending on at least the sensed level of the biomarker. A predetermined threshold for heart rate can be greater than 90, 100, 110, or 120 beats per minute, and when the sensed level exceeds the predetermined threshold, the acute relief therapy can be applied. In some embodiments, the method provides multiple therapy pathways that are at least in part dependent on sensing of user biomarkers (e.g., heart rate, heart rate variability, heart rhythm, skin sympathetic nerve activity, electrodermal activity, body temperature as measured on the wrist, respiratory cycle, brain electrical activity, cytokine levels, physical activity, oxygen levels, etc.). In some embodiments, biomarkers can include patient demographics, prior medication usage, prior device therapy usage, and/or sleep cycles. In some embodiments additional data such as weather information in the patient's local address (e.g., air temperature, humidity, pressure, elevation) can be monitored to impact stimulation and/or treatment. In one embodiment, physical activity can be measured with a motion sensor. In one embodiment, oxygen levels can be measured with a pulse oximeter (e.g., via pulse oximetry). Biomarkers may be sensed immediately prior to, during and/or after therapeutic stimulation. In another embodiment, biomarkers are sensed hours or days prior to or after stimulation. For example, blood tests may be used to determine elevated biomarkers such as cytokines or other inflammatory compounds, and stimulation is applied as a treatment to reduce such biomarkers. In some embodiments, other bodily fluids are used to determine biomarkers, such as urine, saliva, sweat, tears, nasal excretions, etc. These may be measured with sensors that are separate from (e.g., independent from) or in communication with the neuromodulation components described herein.

In some embodiments, a predetermined threshold for heart rate variability can be greater than 1, 2, 3, 4, or 5, and when the sensed level exceeds the predetermined threshold, the neurostimulation (e.g., preventative therapy) can be applied. The predetermined threshold can be determined on a case-by-case basis and are patient specific.

In some embodiments, at least one of the first electrical stimulation or the second electrical stimulation can be delivered in bursts of pulses. In some embodiments, a burst frequency of stimulation can be any value between 0 Hz and 15 Hz or 4 Hz and 12 Hz. In some embodiments, a pulse frequency of stimulation can be any value between 0 Hz and 200 Hz or 50 Hz and 150 Hz.

In some embodiments, the electrical stimulation is delivered in bursts of pulses. In some embodiments, a burst frequency of stimulation can be any value between 0 Hz and 15 Hz or 4 Hz and 12 Hz. In some embodiments, a pulse frequency of stimulation can be any value between 0 Hz and 200 Hz or 50 Hz and 150 Hz. In some embodiments, a burst frequency of stimulation can be any value between 0 Hz and 15 Hz or 1 Hz and 14 Hz. In some embodiments, a pulse frequency of stimulation can be any value between 0 Hz and 150 Hz or 1 Hz and 149 Hz.

In some embodiments, a neuromodulation system is provided. The system (e.g., including one or more devices and/or components) can comprise an electrical stimulation pulse generator that delivers electrical stimulation pulses to an earpiece that is placed inside the ear. The earpiece can comprise two electrodes protruding from a top of a boot that rest on the concha of the ear to stimulate the auricular branch of the vagus nerve and/or other nerves that innervate the ear, one or more sensors that measure data from one or more biomarkers of a user's physiological state, and a controller that receives and uses the measured data to adjust one or more stimulation parameters of the electrical stimulation pulses. In several embodiments, the boot comprises a housing with a material for placement on a portion of skin. The boot may be made at least partially of silicone and designed to rest at an entrance of an ear canal.

In some embodiments, the electrical stimulation pulses can be delivered at a pulse frequency of 1 Hz to 100 Hz. In some embodiments, the electrical stimulation can be delivered continuously. In some embodiments, the electrical stimulation pulse generator can comprise a round form factor and is attached via a clip to some article of clothing. In some embodiments, the electrical stimulation pulse generator can be integrated into the earpiece to comprise a device resembling a hearing aid in appearance.

In some embodiments, the neuromodulation device can further comprise a watch-like wrist worn stimulation device that delivers electrical stimulation to peripheral nerves located in the wrist. The stimulation device can comprise a band containing two rows of three electrodes, where the center electrode in each row is a stimulating electrode and the electrodes on either side of the center electrode are charge balance electrodes. The stimulation device can further comprise a second electrical stimulation pulse generator worn on the wrist that delivers bursts of electrical stimulation pulses to the electrodes on the band, a user interface comprising a display on the face of the watch-like device, and a base station configured to charge and house the watch-like device.

In some embodiments, one of the sensors is a photoplethysmography sensor and the biomarker measured is heart rate or heart rate variability. In some embodiments, one of the sensors is an electrocardiogram and the biomarker is heart rhythm. In some embodiments, the electrocardiogram is integrated into the stimulator of the watch-like device. In some embodiments, the electrocardiogram is integrated into the base station described above. In some embodiments, the electrocardiogram is integrated into a patch to be worn on a user's body.

In some embodiments, one of the sensors described above is a sensor for detecting the biomarker of skin sympathetic nerve activity. In some embodiments, one of the sensors described above is a sensor for detecting electrodermal activity. In some embodiments, one of the sensors described above is a sensor for detecting skin temperature. In some embodiments, one of the sensors is a mechanical sensor integrated into a belt worn around the chest that detects changes in a respiratory cycle.

In some embodiments, electrical stimulation pulse generator communicates wirelessly to a belt respiration sensor and delivers electrical stimulation to the earpiece via a conduit. The earpiece can be a silicone boot that rests at the entrance of the ear canal. The earpiece can be a clip that attaches to the helix of the ear and comprise a reflective or transmissive photoplethysmography sensor.

In some embodiments, one of the sensors is a microphone that is worn in the ear to detect changes in the user's respiratory cycle. In some embodiments, one of the sensors measures the temperature in the ear. In some embodiments, one of the sensors is an infrared reflective light monitor integrated into the earpiece to detect changes in the user's respiratory cycle. In some embodiments, one of the sensors is an electroencephalogram integrated into the earpiece to measure brain activity. In some embodiments, one of the sensors measures cytokine levels in the body, and is integrated into the stimulator device, a skin patch, or a belt.

In some embodiments, the system and methods are used to provide acute therapy for users suffering from migraine, colitis, irritable bowel disease, rheumatoid arthritis, hypertension, an episode of atrial fibrillation, or other cardiac arrhythmias or pathologies. In some embodiments, the system and methods are used to prevent or reduce the severity or frequency of future episodes of atrial fibrillation, or episodes of other cardiac arrhythmias.

In some embodiments, the earpiece can comprise a pressure applicator configured to bias the two electrodes in a direction towards the ear. The earpiece can comprise two electrodes protruding or otherwise extending from the top of a boot In one embodiment. In several embodiments, the boot comprises a housing with a material for placement on a portion of skin. The boot may be made at least partially of silicone and designed to rest at an entrance of an ear canal. The pressure applicator(s) can be configured to increase a level of pressure applied by the two electrodes against the ear so as to lower an impedance between the two electrodes and the ear. The pressure applicator can include for example the following structural and/or functional features: actuating surface(s), such as a spring loaded actuating surface or other pressure application modalities (e.g., gas pressure, fluid pressure, foam pressure, magnetic, and/or temperature variant material pressure configurations). Although pressure applicators are used to increase the contact of the electrode to the skin on or near the ear, they can also be used for the same purpose for electrodes used on other parts of the body such as the arm (e.g., wrist) or leg. In some embodiments, the neuromodulation (e.g., neurostimulation) devices use means to apply pressure, bring an electrode in closer contact with the skin, increase conductivity, decrease impedance, or combinations of these functions.

In some embodiments, a system for determining a respiratory phase of a user is provided. The system can comprise a sensor for detecting and measuring a quantitative value generally relating to the respiratory phase of the user. The quantitative value can be one or more of a respiration threshold, a sample check count, a respiration slope threshold, and a lockout length. The system further includes a controller configured to apply an algorithm to the quantitative value and determine the respiratory phase of the user based on the application of the algorithm to the quantitative value. The quantitative value can be the respiration threshold, and the respiration threshold can be a minimum difference in amplitude between two sample values. The quantitative value can be the sample check count, and the sample check count can be a minimum number of samples in a row required to be checked in order to consider whether the user has switched from one respiratory phase to another. The quantitative value can be the respiration slope, and the respiration slope threshold can be the minimum slope value required to assign a change from one respiratory phase to another. The quantitative value can be the lockout length, and the lockout length can be a minimum amount of time in which the algorithm is paused. The determined respiratory phase can be an inhalation phase or an exhalation phase. The respiratory phase or other respiratory data can then be used, in some embodiments, as a biomarker to begin or ramp up stimulation to one or more nerves.

In some embodiments, a value of a stimulation modality parameter for the first electrical stimulation is different than a value of the stimulation modality parameter for the second electrical stimulation. In some embodiments, a value of a stimulation modality parameter for the first electrical stimulation is the same as a value of the stimulation modality parameter for the second electrical stimulation.

Any one of the devices described herein can be used for the prevention (e.g., reduction of symptoms, and/or treatment of depression (such as post-partum depression), inflammation, (such as neuroinflammation), Lyme disease, neurological diseases (such as Parkinson's and Alzheimer's), and gastrointestinal issues (including those in Parkinson's disease), inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases (such as neuroinflammation), cardiac conditions (such as atrial fibrillation, hypertension, and stroke), epilepsy and/or seizures, headaches (such as migraine), and inflammatory skin conditions and immune dysfunction.

In some embodiments, an auricular device for noninvasive nerve neuromodulation comprises: a first portion configured to secure at least partially within an ear canal of a user; and a second portion coupled with the first portion and configured to be placed adjacent (e.g., next to, within or in contact with) the ear of the user when the first portion is at least partially secured within the ear canal of the user, wherein the second portion comprises a nerve effector (such as one or more electrodes or means to deliver electrical stimulation) configured to modulate one or more nerves in or around the ear. The nerve effector can comprise at least a first and/or second electrode and the nerve effector can be configured to stimulate the vagal nerve. In one embodiment. The first electrode can comprise an active electrode and the second electrode can comprise a return electrode. The active and return electrodes can be spaced apart from one another by a distance that is between approximately 10 mm and approximately 15 mm. In some embodiments, said distance is approximately 10, 10.5, 11, 11.5, 12, 12.5, 13, 13.5, 14, 14.5, and 15 mm. In one embodiment, the range is 11-13 mm. In some embodiments, the nerve effector is configured to exert a normal force on the ear (e.g., at the concha cymba, concha cavum, helix, scapha, anti-helix, triangular fossa, superior crus, inferior crus, helicis crus, tragus, intertragic notch, lobule, and/or anti-tragus, or combinations thereof) when the first portion of the auricular device is at least partially secured within the ear canal of the user. In some embodiments, said normal force is between approximately 0.01 Newton (N) and approximately 1 Newton (N) (e.g., 0.01 to 0.05 N, 0.05 to 0.05 N, 0.01 to 0.1 N, 0.1 to 0.5 N, 0.5 to 1 N, and overlapping ranges therein). In some embodiments, the vagus nerve, trigeminal nerve and/or greater auricular nerve is/are neuromodulated. In some embodiments, only the vagus nerve (such as an auricular branch or non-auricular branch of the vagus nerve) is neuromodulated. In some embodiments, the vagus nerve (such as an auricular branch or non-auricular branch of the vagus nerve) and one, two or more other nerves are neuromodulated (e.g., trigeminal nerve, greater auricular nerve, nerves of the auricular branch, auricular branch of the vagus nerve, the facial nerve, the auriculotemporal nerve etc.). In some embodiments, the vagus nerve (such as an auricular branch or non-auricular branch of the vagus nerve) is not stimulated and instead, for example, another nerve is stimulated (e.g., trigeminal nerve, greater auricular nerve, the facial nerve, the auriculotemporal nerve, other nerves of the auricular branch, etc.). Neuromodulation according to several embodiments includes stimulation using, for example, the parameters disclosed herein. A second therapy (such as vibratory therapy) is provided in conjunction with neurostimulation in one embodiment.

In some embodiments, the first portion can comprise an ear canal element and the second portion comprises at least one prong, and the auricular device can further comprise a stem connecting the ear canal element to the at least one prong. In some embodiments, the nerve effector comprises an active electrode and a return electrode. In some embodiments, the active and return electrodes are positioned along the at least one prong. In some embodiments, the at least one prong comprises a first prong and a second prong, each of the first and second prongs having a first end connected to the stem and a second end opposite the first end, wherein the active electrode is positioned at the second end of the first prong and the return electrode is positioned at the second end of the second prong. In some embodiments, the second ends of the first and second prongs are spaced away from one another. In some embodiments, the second ends of the first and second prongs are spaced away from one another by a distance that is between approximately 10 mm and approximately 15 mm. In some embodiments, said distance is approximately 11-13 mm (e.g., 11.5 mm). In some embodiments, a diameter of an electrode-skin contact surface area of each of the active and return electrodes is between approximately 2 mm and approximately 8 mm (e.g., 2-4, 3-5, 4-6 mm, 6-8 mm, and overlapping ranges therein). In some embodiments, said diameter of said electrode-skin contact surface area of each of the active and return electrodes is approximately 3-5 mm (e.g., 4 mm). In some embodiments, the first and second prongs are angled with respect to one another at an angle between approximately 20° and approximately 90° (e.g., 20°-30°, 30°-40°, 40°-50°, 50°-60°, 60°-70°, 70°-80°, 80°-90°, and overlapping ranges therein). In some embodiments, the first prong has a greater length than the second prong. In some embodiments, the second prong has a greater length than the first prong. In some embodiments, one or more of the prongs are not rotatable and/or not bendable (e.g., relative to the stem). In one embodiment, the boss provides sufficient adjustability (e.g., allowing certain rotation) without the need for prong rotation and/or bendability.

The auricular device can further comprise a boss configured to couple the stem to the ear canal element. In some embodiments, the boss is configured to move relative to the stem while coupled with the stem and the ear canal element. In some embodiments, the boss is configured to allow the ear canal element to rotate relative to the stem while the boss is coupled with the stem and the ear canal element. In some embodiments, the boss comprises a pocket configured to receive the stem. In some embodiments, said pocket surrounds a portion of a perimeter of a cross-section of the stem when the boss is coupled with the stem. In some embodiments, said pocket comprises a protrusion and wherein said stem comprises one or more notches recessed from a surface of the stem, the one or more notches configured to receive at least a portion of the protrusion. In some embodiments, said one or more notches comprises a plurality of notches (e.g., 2, 3, 4, 5, 6 or more notches).

The auricular device can further comprise a power source configured to provide power to the nerve effector. The power source may be, for example, one or more batteries (e.g., rechargeable battery). In one embodiment, the power source is placed on the device coupled to the ear or in a location other than the ear, such as the wrist or other location on or within the body.

In some embodiments: the first portion comprises an ear canal element; the second portion comprises at least one prong; the auricular device further comprises a stem connecting the ear canal element to the at least one prong; and the nerve effector comprises an active electrode and a return electrode. In some embodiments, the at least one prong is flexible. In some embodiments, the at least one prong is resilient. In some embodiments, the at least one prong comprises a first prong and a second prong, each of the first and second prongs having a first end connected to the stem and a second end opposite the first end, wherein the active electrode is positioned at the second end of the first prong and the return electrode is positioned at the second end of the second prong. In some embodiments, the first and second prongs are configured to allow each of the active and return electrodes to simultaneously make independent contact with portions of the ear (e.g., concha cymba). In some embodiments, the first and second prongs are configured to exert a normal force on the ear (e.g., concha cymba, concha cavum, helix, scapha, anti-helix, triangular fossa, superior crus, inferior crus, helicis crus, tragus, intertragic notch, lobule, and/or anti-tragus, or combinations thereof) when the ear canal element is at least partially secured within the ear canal of the user. In some embodiments, said normal force is between approximately 0.01 N and approximately 1 N. In some embodiments, at least one of the first and second prongs are flexible and/or resilient. In some embodiments, only one of the first and second prongs are flexible and/or resilient. In some embodiments, both of the first and second prongs are flexible and/or resilient. In some embodiments, the stem is rigid. In some embodiments, the second ends of the first and second prongs are spaced away from one another. Additional prongs may also be used.

In some embodiments, an auricular device for noninvasive vagal nerve neuromodulation comprises an ear canal element configured to secure at least partially within an ear canal of a user, a boss rotatably coupled with the ear canal element, a stem slidably coupled to the boss and rotatable relative to the ear canal element via the boss, at least one prong coupled to the stem, and a nerve effector coupled to an end of the at least one prong. The stem and the at least one prong can be configured to position the nerve effector within, on or near the ear of the user when the ear canal element is at least partially secured to or within the ear canal of the user.

In some embodiments, an auricular device for noninvasive vagal nerve neuromodulation employing a stimulation burst pattern comprises a first portion configured to secure at least partially within an ear canal of a user, a second portion coupled with the first portion and configured to be placed within the concha cymba of an ear of the user when the first portion is at least partially secured within the ear canal of the user, and one or more hardware processors configured to generate the stimulation burst pattern. The second portion can comprise a nerve effector configured to apply the stimulation burst pattern so as to modulate a vagal nerve of the user.

In some embodiments, the auricular device further comprises one or more sensors (e.g., 2, 3, 4, 5, 6 or more sensors). In some embodiments, the one or more sensors are selected from the group consisting of a Photoplethysmography sensor (PPG), a galvanic skin sensor (GSR), an inertial measurement unit sensor (IMU), a temperature sensor (e.g., for body/skin temperature or ambient temperature), a respiratory sensor, and an Electroencephalography sensor (EEG). In some embodiments, respiration is measured with mechanical, electrical, impedance, acoustic (e.g., microphone), ultrasonic, infrared, or video based measures. In some embodiments, the one or more sensors can be employed to measure response to therapy as well as to calibrate therapy. In some embodiments, the auricular device is configured to electrically connect to a power source separate from the auricular device.

In some embodiments, the devices and methods described here do not include or use one or more of the following features: (i) a hydrogel material adjacent the active electrode and/or the return electrode; (ii) an adhesive material adjacent the active electrode and/or the return electrode; (iii) any percutaneous components; and/or (iv) any implantable components. Some embodiments include batteries and/or cables, while others do not.

Controllers are provided in several embodiments. In some embodiments, the system comprises one or more hardware processors configured to: generate a stimulation waveform for stimulation with one or more electrodes, wherein the stimulation waveform comprises a gradual burst pattern; and apply the stimulation waveform to the one or more electrodes. In some embodiments, the one or more hardware processors are further configured to modify the stimulation waveform based on one or more physiological parameters determined from a physiological sensor selected from the group consisting of a photoplethysmography sensor (PPG), a galvanic skin sensor (GSR), a temperature sensor, and an Electroencephalography sensor (EEG). In some embodiments, the one or more hardware processors are further configured to modify the stimulation waveform based on data determined from an inertial measurement unit sensor (IMU).

In several embodiments, depression (including but not limited to post-partum depression, depression affiliated with neurological diseases, major depression, seasonal affective disorder, depressive disorders, etc.), inflammation (such as neuroinflammation), Lyme disease, neurological diseases (such as Parkinson's and Alzheimer's), and gastrointestinal issues (including those in Parkinson's disease). Inflammatory bowel disease (such as Crohn's disease, colitis, and functional dyspepsia), rheumatoid arthritis, multiple sclerosis, psoriatic arthritis, psoriasis and other inflammatory diseases are treated in several embodiments. Inflammatory skin conditions may also be treated.

In some embodiments, cardiac conditions (such as atrial fibrillation, hypertension, and stroke) are treated in one embodiment. Epilepsy and other seizure disorders are treated in one embodiment. Headache, such as migraine, are treated in other embodiments. The neuromodulation devices, e.g., neurostimulation devices, described herein can be used for the treatment of chronic fatigue syndrome. In one embodiment, a method framework for treating rheumatoid arthritis, atrial fibrillation or migraine with peripheral nerve stimulation comprises a plurality of therapy pathways: (a) an acute relief pathway, and/or (b) a preventative therapy pathway. In one embodiment, the therapy framework and each therapy pathway may be executed by applying peripheral nerve stimulation via a wrist worn neuromodulation device, an auricular neuromodulation device, or any combination of wrist worn devices and auricular devices. Devices to stimulate nerves in the leg are also provided in some embodiments.

In some embodiments, the devices described herein can be used for the treatment of chronic inflammatory symptoms and flare ups. Bradykinesia, dyskinesia, gait dysfunction, dystonia and/or rigidity may also be treated according to several embodiments. In several embodiments, rehabilitation as a result of certain events are treated, for example, rehabilitation from stroke or other cardiovascular events. Systems and methods to reduce habituation and/or tolerance to stimulation in the disorders and symptoms identified herein are provided in several embodiments by, for example, introducing variability in stimulation parameter(s) described herein.

In some embodiments, a system for applying neuromodulation to a subject includes multiple neuromodulation devices placed on or proximate to different portions of a subject's body. For example, such system can include a first neuromodulation device (such as any of the auricular devices described herein) placed on or proximate to a subject's ear and a second neuromodulation device is placed on or proximate to a different portion of the subject's body (such as the wrist, palm, finger, portion of an arm, leg, ankle, foot, sole, toe, etc.). One, two, three or four neuromodulation devices may be worn by a subject. When two or more neuromodulation devices are used, they may be activated separately or together (e.g., synchronized). Modulation of the vagus nerve is accomplished with the devices described herein, according to several embodiments. In some embodiments, the devices described herein are used to stimulate the autonomic system. In some embodiments, the devices described herein are used to balance the sympathetic/parasympathetic systems. In some embodiments, improvement in the condition to be treated is one indicator of that such balancing has occurred. For example, reduction of one or more of tremor, inflammation, cardiac aberrations, imbalance, movement disruptions, headache, pain, etc. post use of the neuromodulation devices described herein (as compared to pre-use) is used, in some embodiments, to indicate balancing the sympathetic/parasympathetic system. Balancing may also be shown by measuring neurotransmitters and showing improvements in neurotransmitter function, amount, activity, uptake, etc.

In some embodiments, neuromodulation with devices and methods described herein (e.g., neuromodulation of the vagal nerve) affects (increases, decreases or maintains) neurotransmitter release, uptake and/or metabolism. Certain neurotransmitters may be increased, while others may be decreased to achieve the desired effect. The dopaminergic and/or serotonergic systems are regulated according to several embodiments described herein. In some embodiments, the brain-gut axis is regulated with the devices and methods described herein.

Although nerve stimulation is disclosed in several embodiments herein, it should be appreciated that downregulation of various pathways may be achieved. For example, cytokine production and/or activity may be inhibited to treat inflammation (including disorders such as various arthritic conditions, gastrointestinal disorders, etc.). Certain neurotransmitter production and/or activity may be decreased. Neurotransmitter uptake and/or metabolism may be increased.

Methods of using the systems described herein are also provided. For example, in some embodiments, a method of modulating a vagal nerve of a subject comprises: generating, with one or more hardware processors, a stimulation waveform for stimulation with one or more electrodes, wherein the stimulation waveform comprises a gradual burst pattern; and applying the stimulation waveform to a portion of the subject's body with the one or more electrodes. In some embodiments, the method further comprises determining one or more physiological parameters of the subject using a physiological sensor and modifying the stimulation waveform based on said one or more physiological parameters. In some embodiments, the method further comprises determining motion data of the subject with an inertial measurement unit sensor (IMU) and modifying the stimulation waveform based on said motion data. In some embodiments, said physiological sensor comprises a photoplethysmography sensor (PPG). In some embodiments, said physiological sensor comprises a galvanic skin sensor (GSR). In some embodiments, said physiological sensor comprises an Electroencephalography sensor (EEG) or a sensor that measures temperature.

In some embodiments, a method of noninvasively modulating a vagal nerve of a subject comprises: positioning a neuromodulation device proximate an ear of the subject; and modulating the vagal nerve of the subject with a nerve effector of the neuromodulation device. In some embodiments, said nerve effector comprises at least one electrode and said modulating the vagal nerve of the subject comprises stimulating the vagal nerve with said at least one electrode. In some embodiments, said nerve effector comprises at least a first and second electrode. In some embodiments, said first electrode comprises an active electrode and said second electrode comprises a return electrode. In some embodiments, said neuromodulation device comprises a first portion and a second portion coupled with said first portion, wherein said second portion comprises the nerve effector, and wherein the method further comprises securing the first portion at least partially within an ear canal of the subject. Additional electrodes may also be used.

In some embodiments: said first portion comprises an ear canal element; said second portion comprises at least one prong configured to operably position the nerve effector adjacent (e.g., next to, within or in contact with) a portion of the ear of the subject; said neuromodulation device further comprises a stem connecting the ear canal element to the at least one prong; and said method further comprises adjusting a position of the ear canal element relative to the stem. In some embodiments, said adjusting the position of the ear canal element relative to the stem comprises moving the ear canal element along a portion of a length of the stem. In some embodiments, said neuromodulation device further comprises a boss configured to couple the ear canal element to the stem and allow the ear canal element to move along the portion of the length of the stem. In some embodiments, said ear canal element is rotatably coupled to the stem and wherein said adjusting the position of the ear canal element relative to the stem comprises rotating the ear canal element. In some embodiments, said neuromodulation device further comprises a boss configured to rotatable couple the ear canal element to the stem and allow the ear canal element to rotate while coupled to the stem.

In some embodiments, the method further comprises positioning the nerve effector adjacent (e.g., next to, within or in contact with) the concha cymba or other portions of the ear of the subject, such as the concha cavum, helix, scapha, anti-helix, triangular fossa, superior crus, inferior crus, helicis crus, tragus, intertragic notch, lobule, and/or anti-tragus, or combinations thereof. In some embodiments, first and second nerve effectors (e.g., electrode or other means to deliver electrical pulses or energy) are spaced apart from one another by a distance. In some embodiments, said distance is between approximately 10 mm and approximately 15 mm. In some embodiments, said distance is approximately 11.5 mm. In some embodiments, the method further comprises applying a normal (e.g., perpendicular) force in or around the ear with the nerve effector. In some embodiments, said normal force is between approximately 0.01 N and approximately 1 N.

In some embodiments, vagal nerve activity is down regulated, up regulated or both (e.g., balanced) using the devices described herein. In various embodiments, the devices described herein can be used to apply neuromodulation of the vagal nerve to increase neurotransmitter release, uptake and/or metabolism. In some embodiments, neuromodulation is used to affect (e.g., decrease or increase) neurotransmitter release, uptake and/or metabolism. Several embodiments are used to apply neuromodulation of the vagal nerve to balance neurotransmitter release, uptake and/or metabolism by both increasing and decreasing neurotransmitter activity. Several embodiments are used to apply neuromodulation of the vagal nerve to activate or down regulate the dopaminergic system and/or serotonergic system. Several embodiments are used to apply neuromodulation of the vagal nerve to regulate the brain-gut axis. Several embodiments are used for the treatment of depression (including but not limited to post-partum depression, depression affiliated with neurological diseases, major depression, seasonal affective disorder, depressive disorders, etc.), inflammation (such as neuroinflammation), Lyme disease, neurological diseases (such as Parkinson's and Alzheimer's), and gastrointestinal issues (including those in Parkinson's disease. Several embodiments are used for the treatment of inflammatory bowel disease (such as Crohn's disease, colitis, and functional dyspepsia), rheumatoid arthritis, multiple sclerosis, psoriatic arthritis, osteoarthritis, psoriasis and other inflammatory diseases. In several embodiments, the devices described herein can be used for the treatment of inflammatory skin conditions; chronic fatigue syndrome; and/or chronic inflammatory symptoms and flare ups. In some embodiments, modulation of the blood vessel (either dilation or constriction) is provided using the devices and methods described herein (e.g., through nerve stimulation). Such therapy may, in turn, reduce inflammation (including but not limited to inflammation post microbial infection). The devices and methods described herein can, in various embodiments, increase, decrease or otherwise balance vasodilation and vasoconstriction through neuromodulation (such as modulation of the vagus nerve, trigeminal nerve and/or other nerves in or surrounding the ear). For example, reduction of vasodilation is provided in several embodiments to treat or prevent migraine or other conditions that are aggravated by vasodilation. In other embodiments, vasoconstriction is reduced in, for example, conditions in which dilation is beneficial (such as with high blood pressure and pain). In one embodiment, reduction in inflammation treats tinnitus. In some embodiments, modulation of the blood vessel (either dilation or constriction) is used to treat tinnitus. Tinnitus may be treated according to several embodiments through modulation (e.g., stimulation) of the vagus nerve (such as a non-auricular portion of the vagus nerve) alone or in conjunction with one, two or more other nerves (including for example the trigeminal nerve, greater auricular nerve, nerves of the auricular branch, auricular branch of the vagus nerve, facial nerve, the auriculotemporal nerve, etc.). In one embodiment, nerves other than the vagus nerve are modulated to treat tinnitus. Cranial/auditory nerves may be modulated to treat tinnitus and/or auricular inflammation in some embodiments.

Neuromodulation such as nerve stimulation, as described in several embodiments herein, can provide therapeutic benefit across a variety of diseases, including but not limited to movement disorders (including but not limited to essential tremor, Parkinson's tremor, orthostatic tremor, and multiple sclerosis), urological disorders, gastrointestinal disorders, cardiac diseases, inflammatory diseases (such as neuroinflammation), mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, and stroke) are treated in one embodiment. Epilepsy and other seizure disorders are treated in one embodiment. Inflammatory skin conditions and immune dysfunction are also treated in some embodiments.

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (including but not limited to neuroinflammation and other inflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation (e.g., neuroinflammation, etc.), headache, back pain, joint pain and stiffness, muscle pain and tension, etc.

In several embodiments, devices described herein can be used for the treatment of cardiac conditions (such as atrial fibrillation, hypertension, and stroke). Epilepsy and other seizure disorders are treated in one embodiment. In several embodiments, devices described herein can be used for the treatment of immune dysfunction. Several embodiments are used to stimulate or otherwise modulate the autonomic nervous system and more specifically treat diseases or disease symptoms exacerbated by autonomic dysfunction, including but not limited to depression, anxiety, insomnia, hypertension, cardiac arrhythmia, overactive bladder, inflammatory bowel diseases (e.g., Crohn's disease, colitis, and functional dyspepsia), fecal incontinence, headaches and migraine, chronic pain, vagal syncope, inflammatory diseases (e.g., rheumatoid arthritis, lupus, and other autoimmune diseases) and tinnitus. Several embodiments are used to balance the sympathetic/parasympathetic nervous systems and more specifically treat diseases associated with imbalance of the autonomic nervous system, including but not limited to tremor, a cardiac disorder, a mental health disorder, or another disease or condition such as those disclosed elsewhere herein using a wearable device.

Other disorders can also be treated using the embodiments described herein. For example, stimulation of the vagus nerve has been shown to improve symptoms of hypertension, dexterity, and cardiac dysrhythmias.

For purposes of summarizing the disclosure, certain aspects, advantages, and novel features are discussed herein. It is to be understood that not necessarily all such aspects, advantages, or features will be embodied in any particular embodiment of the disclosure. The disclosure herein supports a myriad of combinations of such aspects, advantages, or features.

### BRIEF DESCRIPTION OF THE DRAWINGS

Certain features of this disclosure are described below with reference to the drawings. The illustrated embodiments are intended to illustrate, but not to limit, the embodiments. Various features of the different disclosed embodiments can be combined to form further embodiments, which are part of this disclosure.

FIG. 1A illustrates an ear of a user in accordance with aspects of this disclosure.

FIG. 1B schematically illustrates an example contact locations for electrodes for delivering nerve stimulation in an ear in accordance with aspects of this disclosure.

FIGS. 1C-1F illustrate embodiments of an electrode ring interface in accordance with aspects of this disclosure.

FIGS. 1G-1K illustrate an experimental set up to measure applied force or pressure and its relation to impedance according to various embodiments of the present disclosure.

FIGS. 1L-1M illustrate electrodes with pressure applicators according to various embodiments of the present disclosure.

FIG. 2A illustrates a perspective view of an embodiment of an auricular device for delivering nerve stimulation in accordance with aspects of this disclosure.

FIGS. 2B-2C illustrate enlarged perspective views of the auricular device of FIG. 2A in accordance with aspects of this disclosure.

FIGS. 2D-2E illustrate side views of the auricular device of FIG. 2A in accordance with aspects of this disclosure.

FIGS. 2F-2G illustrate top and bottom views of the auricular device of FIG. 2A in accordance with aspects of this disclosure.

FIGS. 2H-2J illustrate a portion of the auricular device of FIG. 2A in accordance with aspects of this disclosure.

FIGS. 2K-2M illustrate a portion of the auricular device of FIG. 2A in accordance with aspects of this disclosure.

FIGS. 2N-20 illustrate back and front views of the auricular device of FIG. 2A in accordance with aspects of this disclosure.

FIG. 3 illustrates another embodiment of an auricular device for delivering nerve stimulation in accordance with aspects of this disclosure.

FIG. 4 illustrates embodiments of bosses that can be incorporated into any of the auricular devices disclosed herein.

FIGS. 5A-5B illustrate another embodiment of an auricular device for delivering nerve stimulation in accordance with aspects of this disclosure.

FIGS. 50-5D illustrate how the auricular device of FIGS. 5A-5B can be adjusted for compatibility with different ear anatomies.

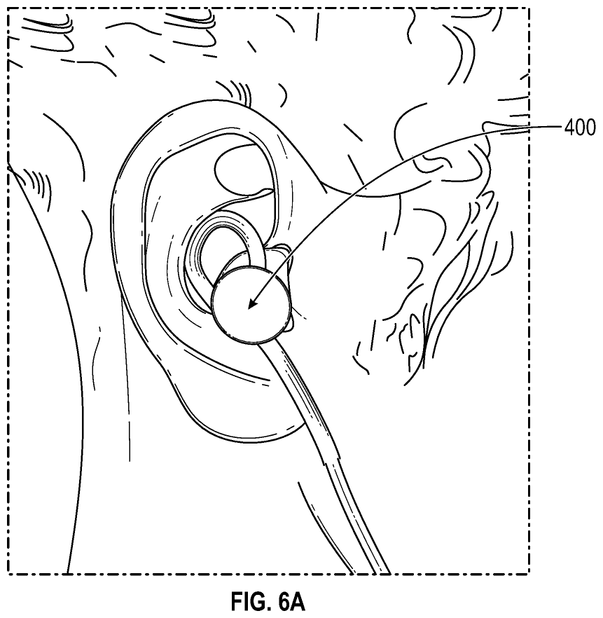

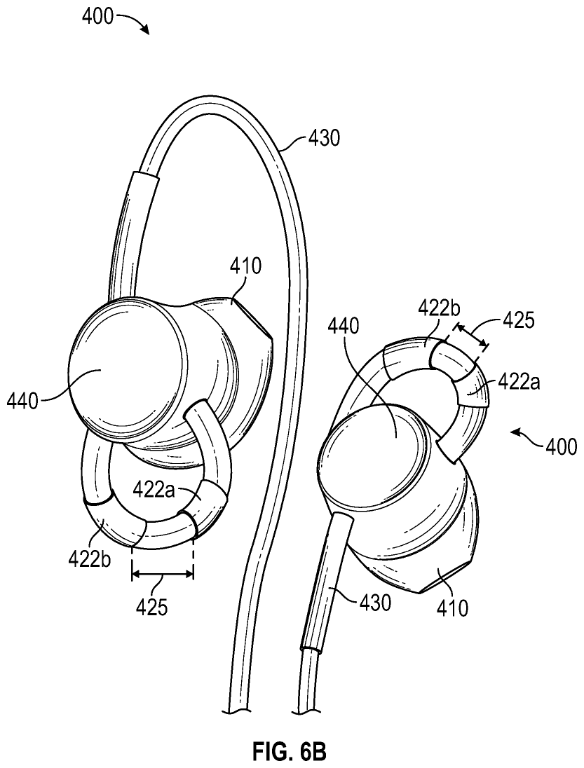

FIGS. 6A-6B illustrate another embodiment of an auricular device for delivering nerve stimulation in accordance with aspects of this disclosure.

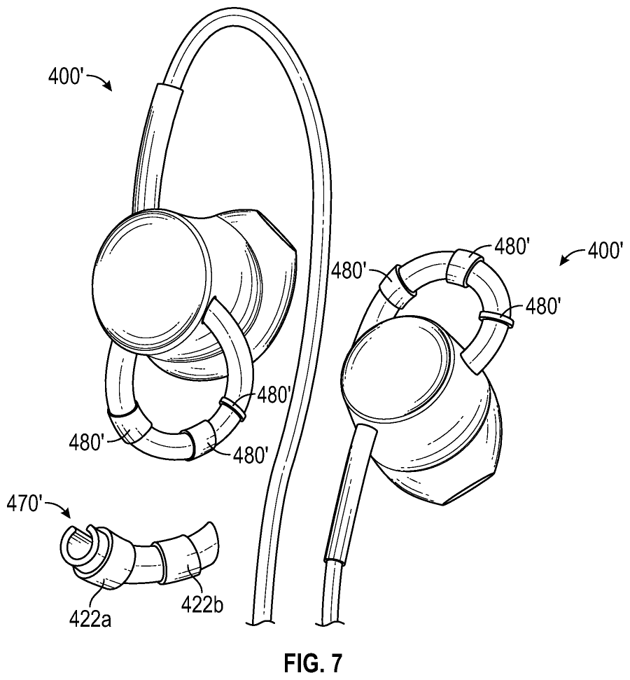

FIG. 7 illustrates another embodiment of an auricular device for delivering nerve stimulation in accordance with aspects of this disclosure.

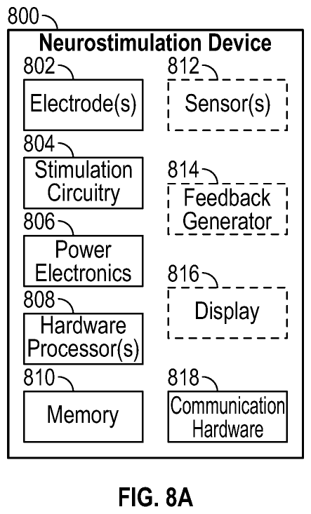

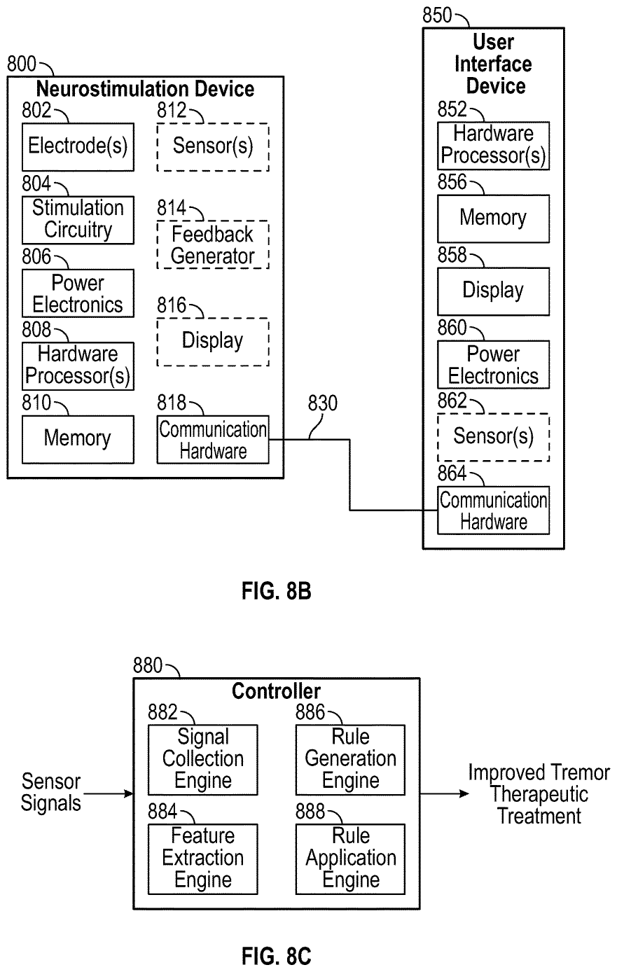

FIG. 8A illustrates a block diagram of an example neuromodulation (e.g., neurostimulation) device.

FIG. 8B illustrates a block diagram of a user interface device that can be connected with the neurostimulation device of FIG. 8A.

FIG. 8C illustrates a block diagram of an embodiment of a controller that can be implemented with some or all the hardware components described with respect to FIG. 8A or 8B.

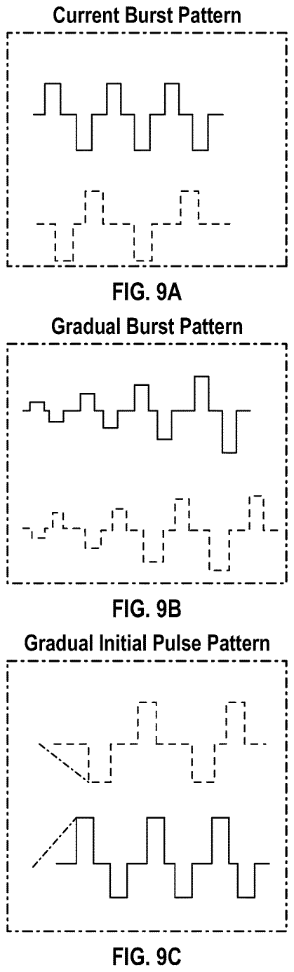

FIGS. 9A-9C illustrates example stimulation patterns that can be applied with the neuromodulation device of FIG. 8A.

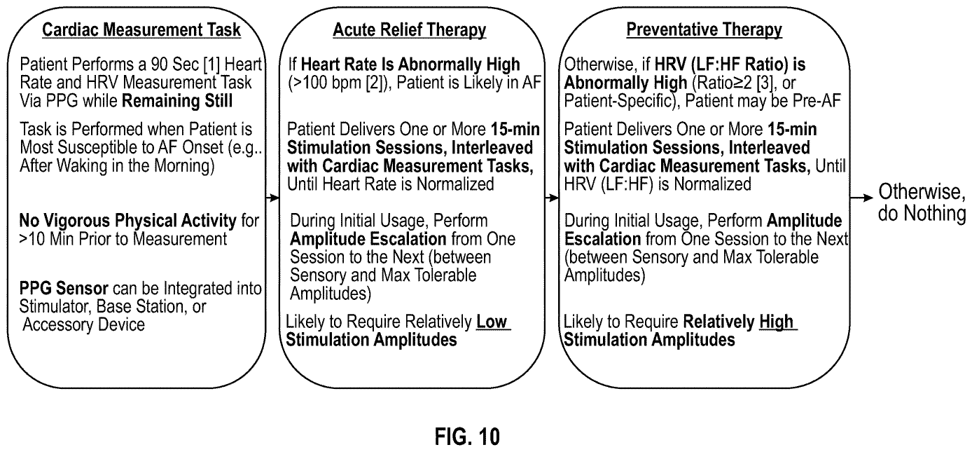

FIG. 10 illustrates a framework that involves a cardiac measurement task, an acute relief therapy, and a preventative therapy according to an embodiment of the disclosure.

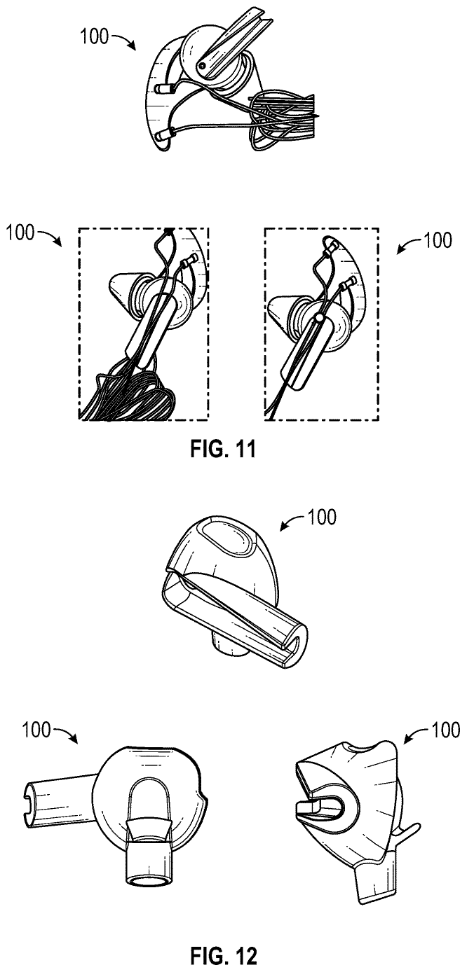

FIGS. 11-12 illustrate neuromodulation devices that deliver electrical stimulation to the auricular branch of the vagus nerve according to embodiments of the disclosure.

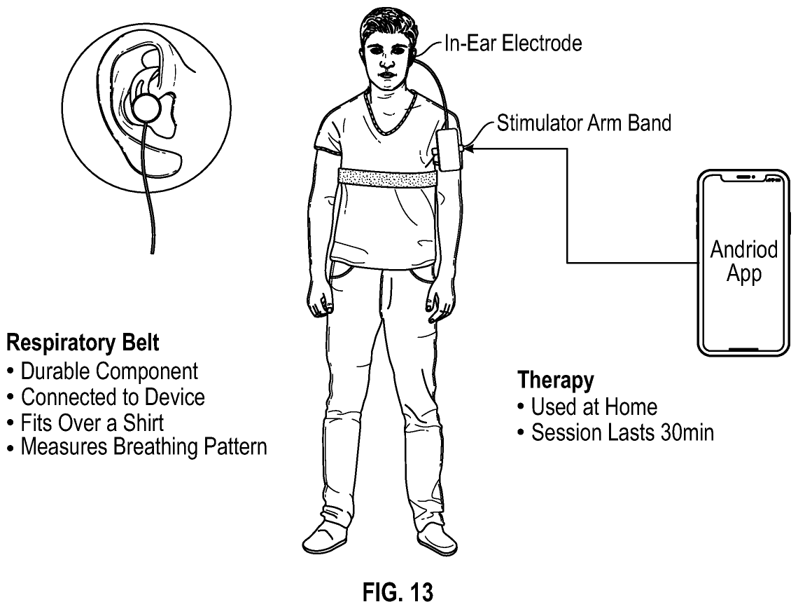

FIG. 13 illustrates devices that deliver electrical stimulation to the auricular branch of the vagus nerve with an additional stimulation device according to embodiments of the disclosure.

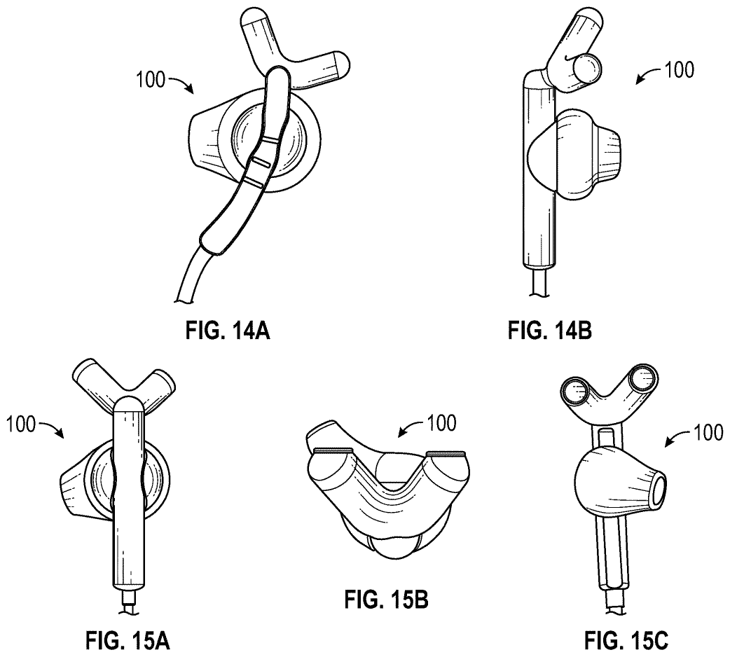

FIGS. 14A-14B illustrate neuromodulation devices that deliver electrical stimulation to the auricular branch of the vagus nerve according to embodiments of the disclosure.

FIGS. 15A-15C illustrate neuromodulation devices that deliver electrical stimulation to the auricular branch of the vagus nerve according to embodiments of the disclosure.

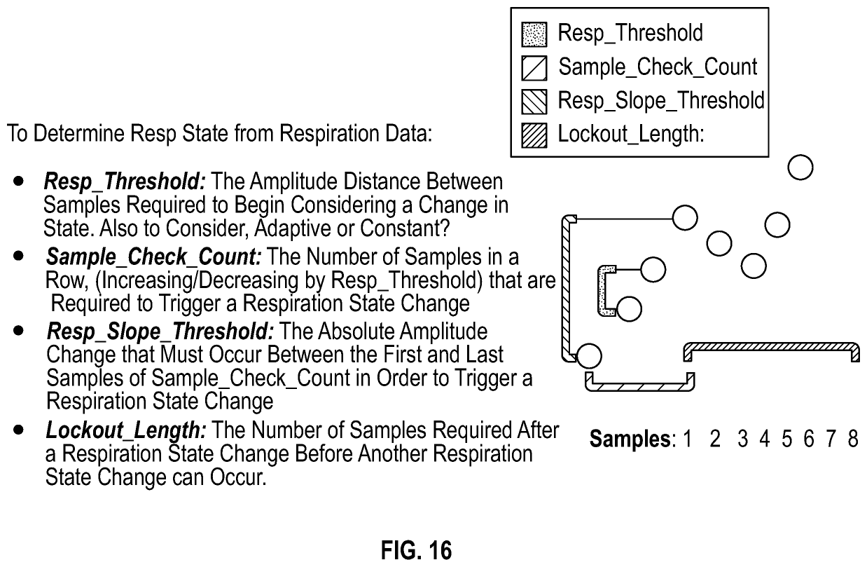

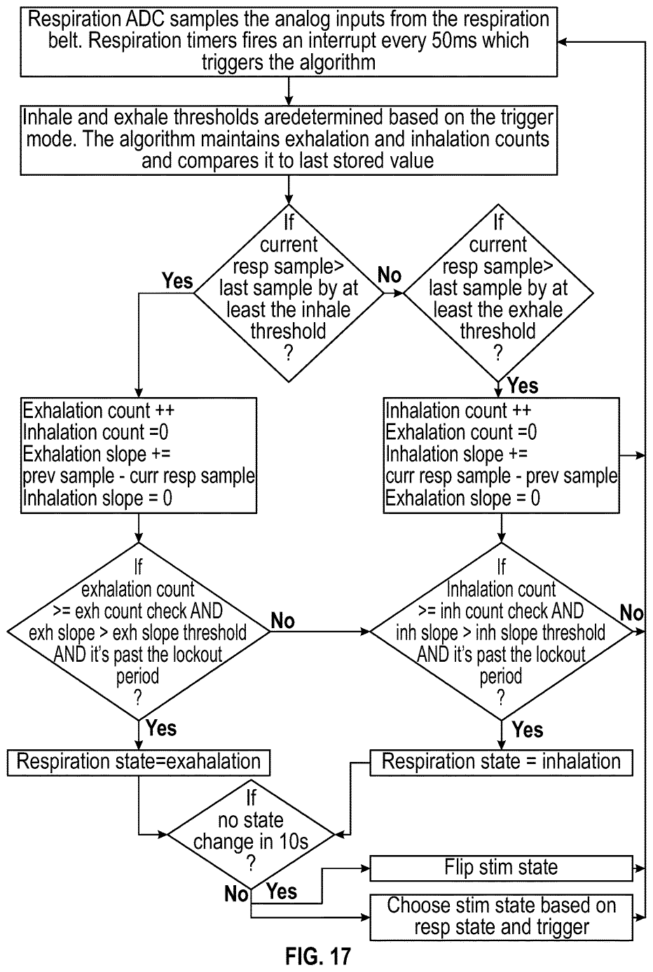

FIGS. 16 and 17 illustrate algorithms for determining a person's current respiratory phase and when a respiratory phase begins or ends according to embodiments of the disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed embodiments and/or uses and obvious modifications and equivalents thereof. Thus, it is intended that the scope of this disclosure should not be limited by any particular embodiments described below. The features of the illustrated embodiments can be modified, combined, removed, and/or substituted.

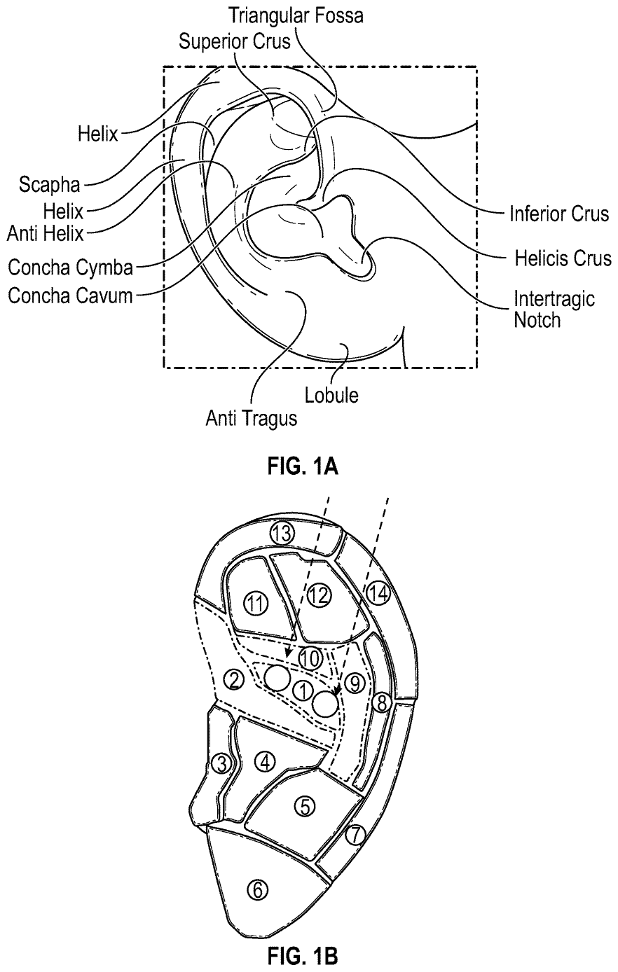

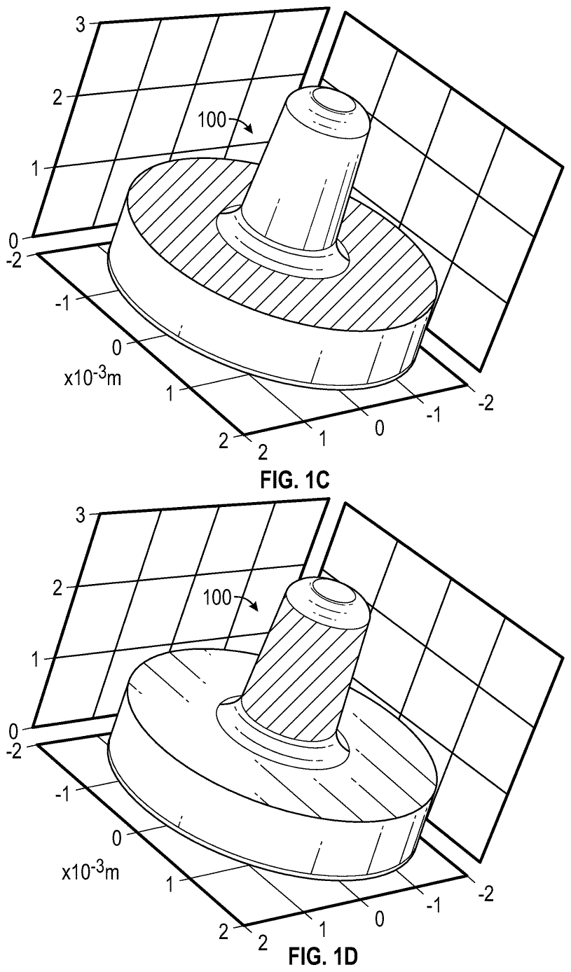

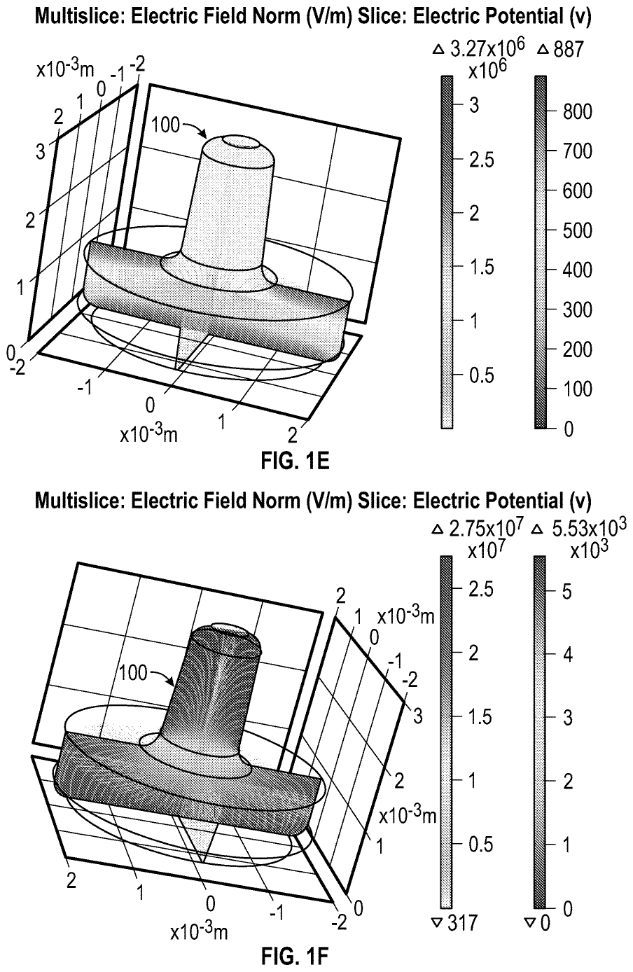

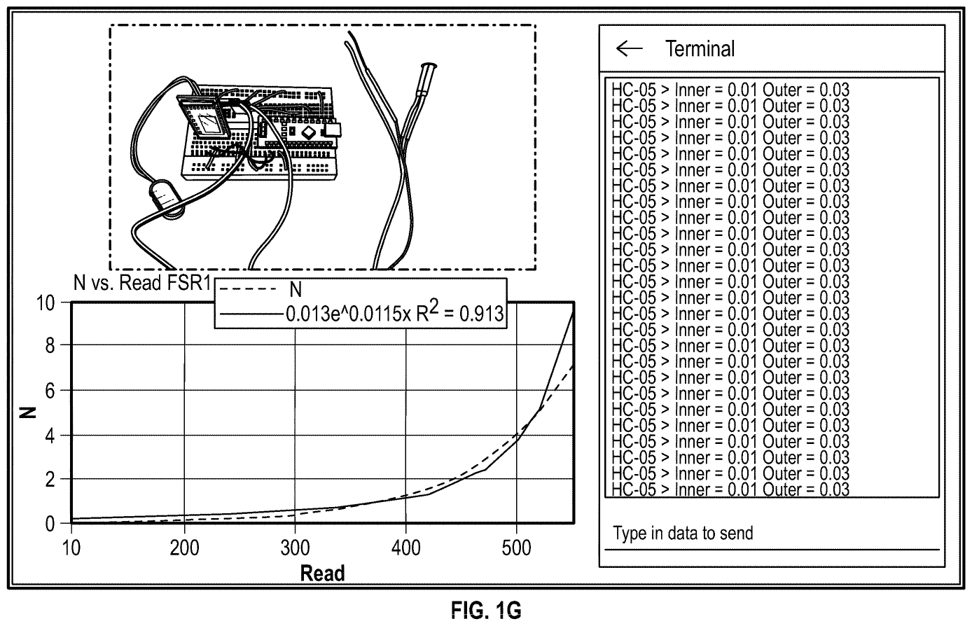

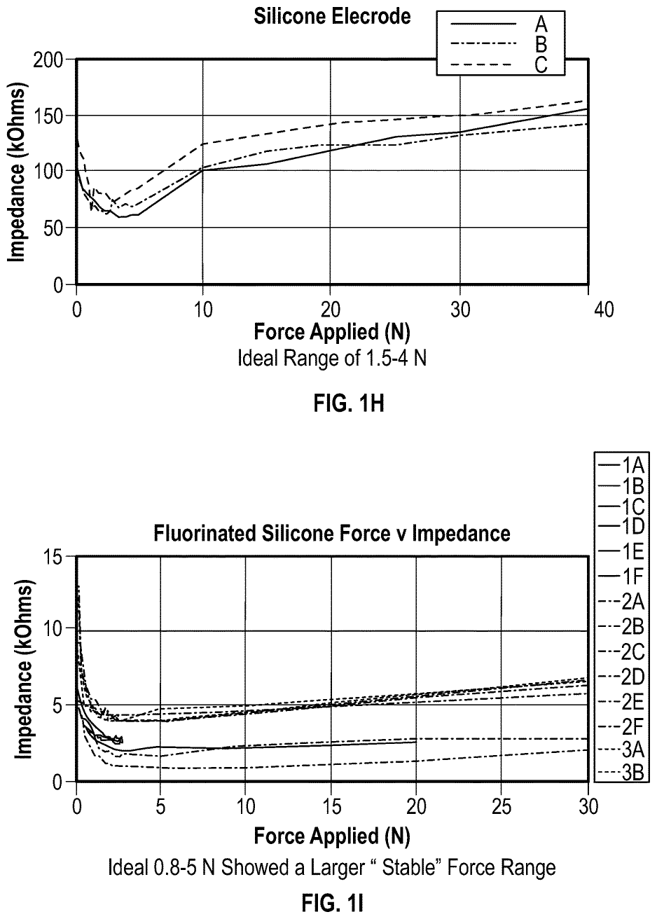

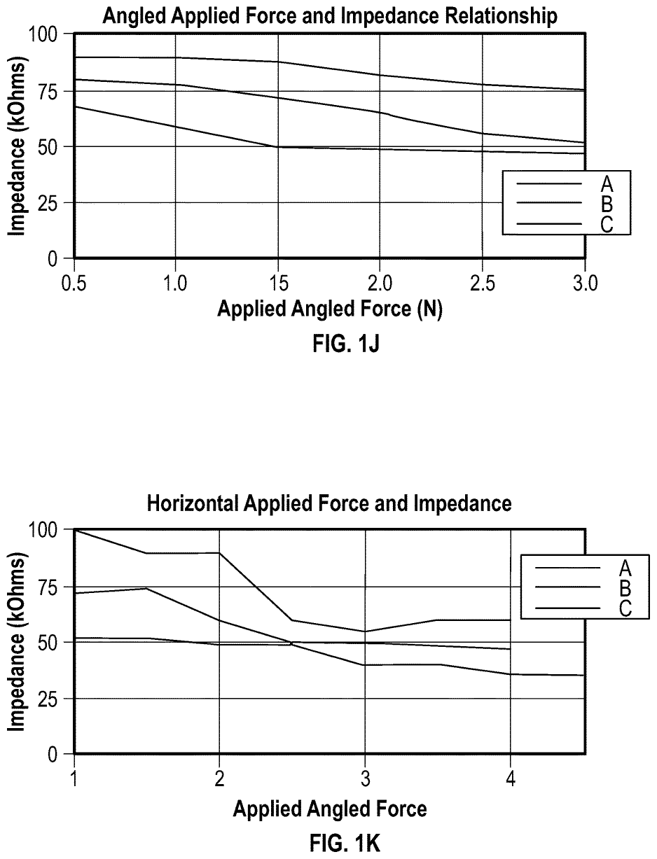

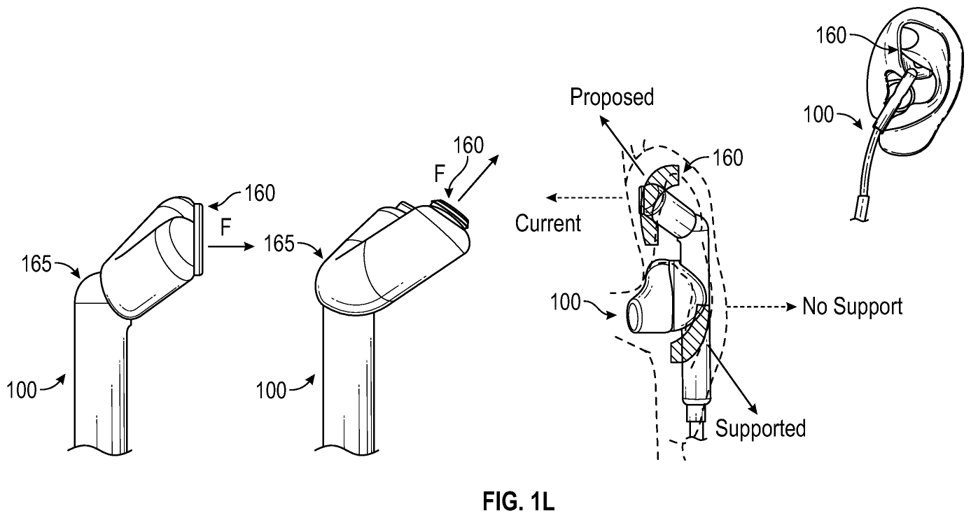

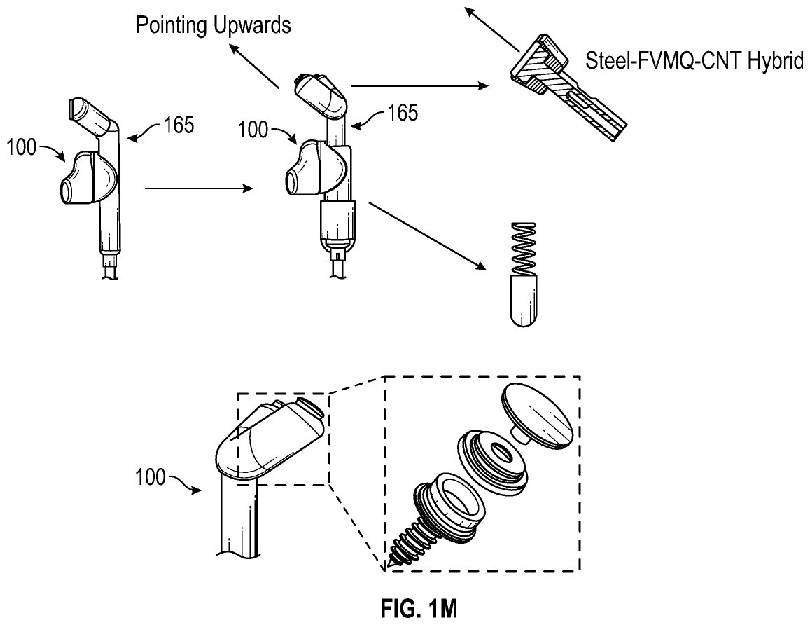

A number of disorders and conditions, including but not limited to, post-partum depression and gastrointestinal issues in Parkinson's disease can be treated by vagal nerve stimulation. FIG. 1A illustrates an example ear with various portions indicated with text, including, one or more of the helix, scapha, anti-helix, triangular fossa, superior crus, inferior crus, helicis crus, tragus, intertragic notch, lobule, anti-tragus, and concha (e.g., concha cavum and/or concha cymba). Due to the location of termination of the vagal nerve, in one embodiment, an appropriate region of the ear for stimulation is within the concha cymba. Other locations, such as a helix, scapha, anti-helix, triangular fossa, superior crus, inferior crus, helicis crus, tragus, intertragic notch, lobule, anti-tragus, and/or concha cavum of the ear, and combinations thereof, are also used in various embodiments. Modulation of the vagus nerve is accomplished with the devices described herein, according to several embodiments. In some embodiments, the devices described herein are used to stimulate the autonomic system. In some embodiments, the devices described herein are used to balance the sympathetic/parasympathetic systems (e.g., by upregulating/downregulating/maintaining nerve activity to achieve balance). Several embodiments of the system leverage multiple elements to vary therapy to prevent habituation and/or adjust amplitude to manage discomfort. In one embodiment, varying frequency or other parameters reduces tolerance or habituation and/or increase patient comfort/compliance.

FIG. 1B illustrates two example locations where nerve effectors (e.g., electrodes) can be placed within the concha cymba in order to deliver stimulation. In several embodiments, placement to a helix, scapha, anti-helix, triangular fossa, superior crus, inferior crus, helicis crus, tragus, intertragic notch, lobule, anti-tragus, concha cavum, and/or concha cymba of the ear, or combinations thereof are provided. Some embodiments of the auricular devices discussed herein include electrodes (e.g., an active and a return electrode) that are positioned in locations such as that shown FIG. 1B. In one embodiment, an electrode includes stainless steel, which has low impedance but needs gel for electrical coupling to the skin/tissue. In one embodiment, an electrode comprises a base material (e.g., silicone) and a conductive filler or loading material (e.g., carbon nanotubes (CNT)). In some embodiments, the conductive filler material may include a powder or fine particulate material. The conductive filler material may include metal, carbon, or a mixture thereof. In some embodiments, the conductive filler material may include single wall carbon nanotubes (SWCN). In some embodiments, the conductive filler material may include double wall carbon nanotubes (DWCN). In certain embodiments, loading material in the form of CNT does not require coupling gel, but has a high base line impedance and can have a rapid deterioration in performance with wear and use.

In one embodiment, illustrated at FIGS. 1C-1D and FIGS. 1E-1F, auricular device 100 includes an electrode 122 that includes a ring interface. In one embodiment, the width and thickness is varied. In one embodiment, a nub less design is used.

In one embodiment, impedance drops with greater pressure on the interface between the electrode and the skin/tissue. In one embodiment, an optimal pressure impedance is achieved with a pressure of ~ 2-3 Newtons (N). In various embodiments, 0.01 Newton (N) and approximately 1 Newton (N) (e.g., 0.01 to 0.05 N, 0.05 to 0.05 N, 0.01 to 0.1 N, 0.1 to 0.5N, 0.5 to 1 N, and overlapping ranges therein). In various embodiments, pressure impedance is achieved with a pressure of ~ 0.01-5 Newtons, (e.g., 0.01, 0.05, 0.1, 0.2, 0.3, 0.4, 0.5, 0.6, 0.7, 0.8, 0.9, 1.0, 1.2, 1.4, 1.5, 1.6, 1.8, 2.0, 2.2, 2.4, 2.5, 2.6, 2.8, 3.0, 3.2, 3.4, 3.5, 3.6, 4.0, 4.3, 4.5, 4.7, 5.0 Newtons and any values and ranges therein). In one embodiment, pressure is measured using an Arduino Nano with FSR, a HC-05 Bluetooth module with Android app to view terminal, and reveals an exponential regression calibration curve to give the reading in Newtons, as illustrated at FIGS. 1G, 1H, 1I, 1J, 1K. In various embodiments, as illustrated at FIGS. 1L-1M, an earpiece includes a pressure applicator 160 (e.g., spring loaded actuating surface, and other pressure application surfaces, e.g., gas pressure, fluid pressure, foam pressure, magnetic, or temperature variant material pressure configurations) and various angles to lower impedance between an electrode and the tissue. In some embodiments, the auricular device 100 can comprise a pressure applicator 160 configured to bias one or more electrodes in a direction towards the ear. The pressure applicator 160 can be configured to increase a level of pressure applied by the one or more electrodes against the ear so as to lower an impedance between the one or more electrodes and the ear. The pressure applicator 160 can be a spring loaded actuating surface. In various embodiments, the pressure applicator 160 involves gas pressure, fluid pressure, foam pressure, magnetic, or temperature variant material pressure configurations. In some embodiments, a neuromodulation system is provided (which includes devices and components). The system can comprise an electrical stimulation pulse generator that delivers electrical stimulation pulses to an earpiece that is placed inside the ear. The earpiece can comprise two electrodes protruding from the top of a boot 165 that rest on the concha of the ear to stimulate the auricular branch of the vagus nerve and a pressure applicator configured to bias at least one of the two electrodes in a direction towards the ear. In several embodiments, the boot 165 comprises a housing with a material for placement on a portion of skin. The boot 165 may be made at least partially of silicone and designed to rest at an entrance of an ear canal. In some embodiments, the pressure applicator can be configured to increase a level of pressure applied by the at least one of the two electrodes against the ear so as to lower an impedance between the at least one or more two electrodes and the ear. The pressure applicator 160 can be a spring loaded actuating surface. In various embodiments, the pressure applicator 160 involves gas pressure, fluid pressure, foam pressure, magnetic, or temperature variant material pressure configurations.

In one embodiment, a loaded dry electrode comprises a base material and a loading material. The base material can be comprised of a silicone or silicone-like material, such as fluorosilicone, but any other elastomer may be used. The loading material can be comprised of carbon nanotubes or any other conductive metallic nanowire. Other filler materials may be used in place of these nanowires or in addition to the nanowires. This loading material may be loaded so that the percent loading into the material matrix ranges anywhere from 0-25% (e.g., 1%, 2%, 5%, 8%, 10%, 12%, 15%, 17%, 19%, 20%, 22%, 24% and/or 25% and any values or ranges therein) . . . . The electrode may be in the range of 0.25-5 millimeters (e.g., 0.25, 0.50, 0.75, 1.0, 1.5, 2.0, 2.5, 3.0, 3.5, 4.0, 4.5, 5.0 and any values or ranges therein). In one embodiment, the electrode is self-wetting.

In one embodiment, an electrode is configured to be a noninvasive neuromodulation device. In one embodiment, the electrode includes an elastomer base that forms a matrix for which a filler material may be loaded. In one embodiment, a dry electrode allows for long-term use for noninvasive peripheral nerve stimulation. In various embodiments, this material can be implemented into electrodes for a wrist-worn device and/or an auricular device.

In one embodiment, a dry electrode comprising a base material and a loading material. In one embodiment, the base material may be one or more of silicone, fluorosilicone, or some other elastomer. In one embodiment, the loading material may be one or more of carbon nanotubes (CNT), metallic nanowires, or other filler materials. In one embodiment, the electrode thickness is at least 0.25 millimeters and at most 5 millimeters. In one embodiment, the electrodes are self-wetting. In one embodiment, the percent loading of the loading material is between 0% and 25%.

Various embodiments of the auricular devices discussed herein can include active and return electrodes that are positioned (for example, center-to-center relative to one another) between approximately 5 mm and approximately 20 mm from one another, for example, between approximately 6 mm and approximately 19 mm, between approximately 7 mm and approximately 18 mm, between approximately 8 mm and approximately 17 mm, between approximately 9 mm and approximately 16 mm, between approximately 10 mm and approximately 15 mm, between approximately 11 mm and approximately 14 mm, between approximately 12 mm and approximately 13 mm, or between approximately 11 mm and approximately 12 mm, or any value therebetween, or any range bounded by any combination of these values, although values outside these values or ranges can be used in some cases. As another example, various embodiments of the auricular devices discussed herein can include active and return electrodes that are positioned approximately 11.5 mm from one another. Such configurations can advantageously maximize vagal nerve stimulation and can accommodate a high variations in ear anatomy and/or characteristics.

Various embodiments of the auricular devices discussed herein can include electrodes (e.g., an active and a return electrode) and the diameter of the electrode-skin contact surface area of each of the electrodes is between approximately 1 mm and approximately 10 mm, for example, between approximately 2 mm and approximately 9 mm, between approximately 3 mm and approximately 8 mm, between approximately 4 mm and approximately 7 mm, between approximately 5 mm and approximately 6 mm, between approximately 3 mm and approximately 5 mm, or between approximately 2 mm and approximately 6 mm, or any value therebetween, or any range bounded by any combination of these values, although values outside these values or ranges can be used in some cases. As another example, various embodiments of the auricular devices discussed herein include electrodes and the diameter of the electrode-skin contact surface area of each of the electrodes is approximately 4 mm. Such configurations can advantageously maximize vagal nerve stimulation and can accommodate a high variations in ear anatomy and/or characteristics.

Various embodiments of the auricular devices discussed herein include a first portion that can secure to and/or within (for example, at least partially within) an ear canal of a user and a second portion coupled with the first portion that can be placed and/or positioned proximate and/or adjacent (e.g., next to, within or in contact with) the concha cymba of an ear of the user when the first portion is secured to and/or within the ear canal. Such second portion can include a nerve effector that can provide electrical neuromodulation (e.g., stimulation). As discussed elsewhere herein, the nerve effector can be and/or include one or more, a plurality of, and/or at least one or at least two electrodes (e.g., an active and a return electrode). Such configurations “linking” the first and second portions of the auricular device together (for example, with a reference point of the ear canal) advantageously provide increased spatial understanding and proper placement of the nerve effector proximate and/or adjacent (e.g., next to, within or in contact with) the concha cymba since the first portion can be utilized as a locating and/or confirmation feature for placement of the auricular device. In several embodiments, placement adjacent a helix, scapha, anti-helix, triangular fossa, superior crus, inferior crus, helicis crus, tragus, intertragic notch, lobule, anti-tragus, concha cavum, and/or concha cymba, or combinations thereof are provided.

Various embodiments of the auricular devices discussed herein can be configured to not interfere with a user's ability to hear external sounds or interfere with a user's ability to connect into personalized audio.

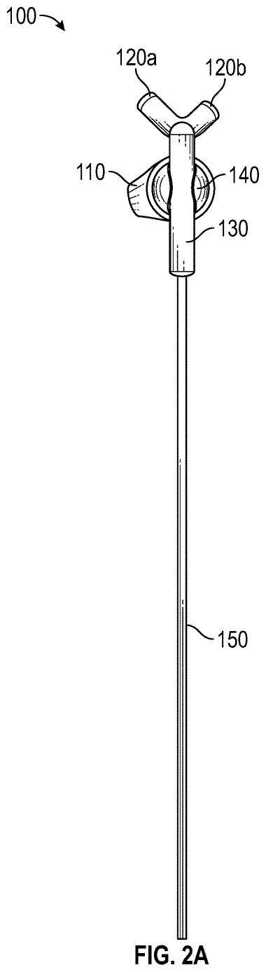

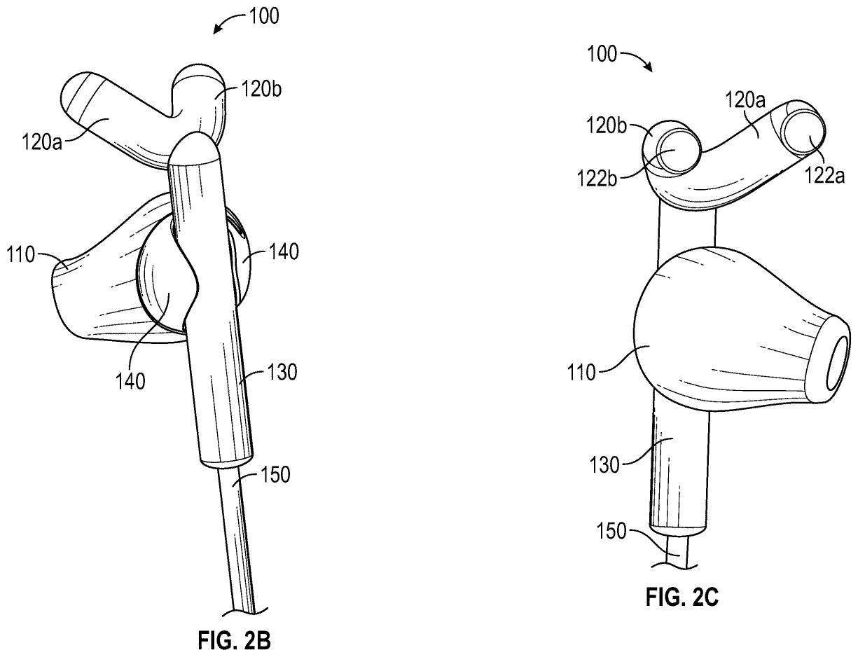

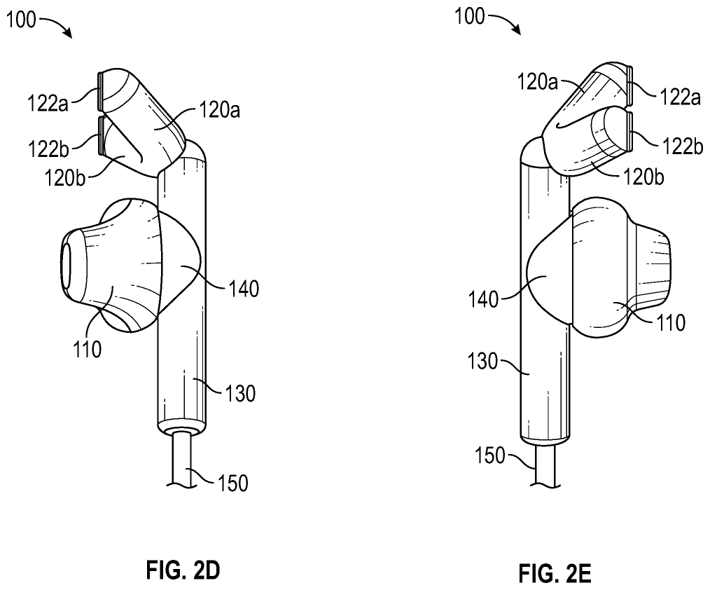

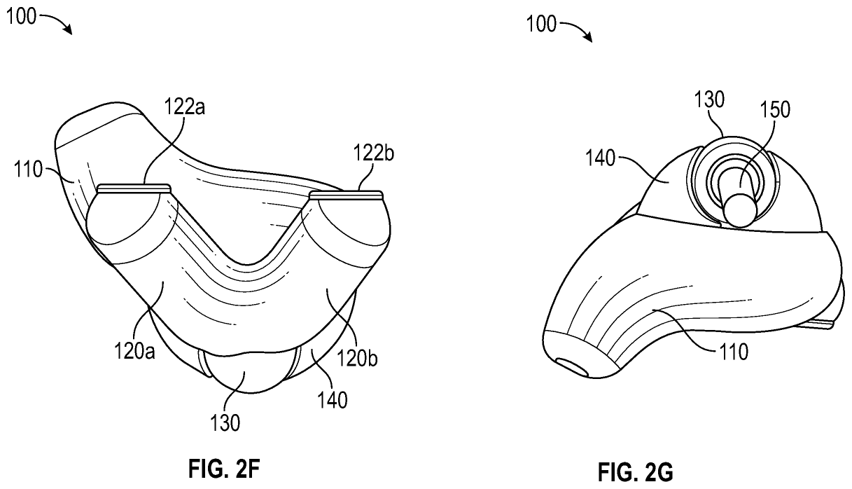

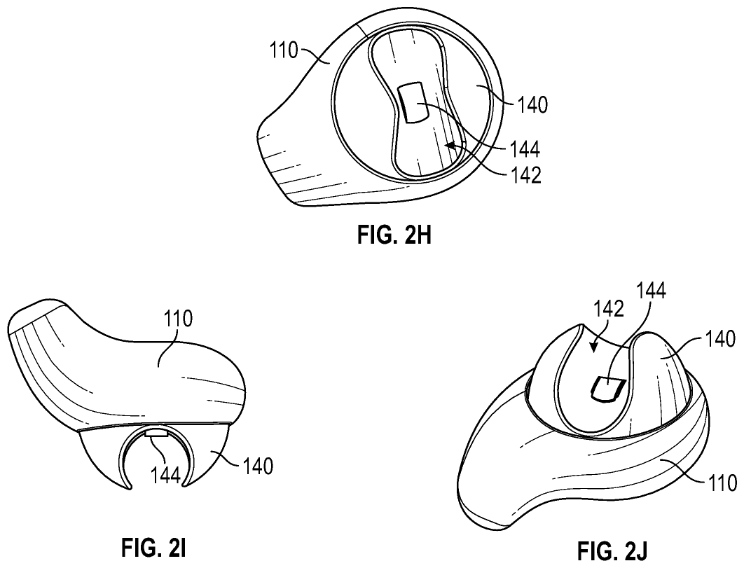

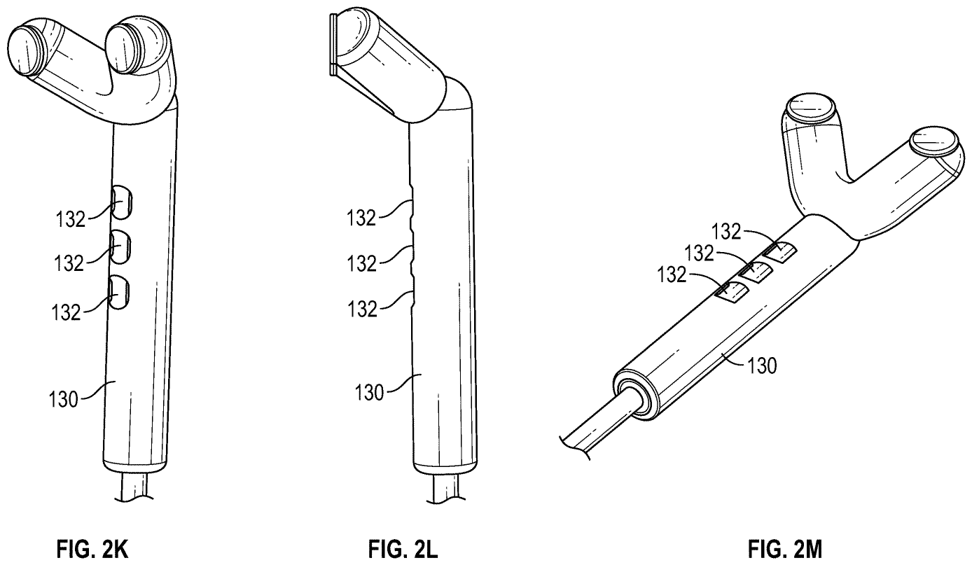

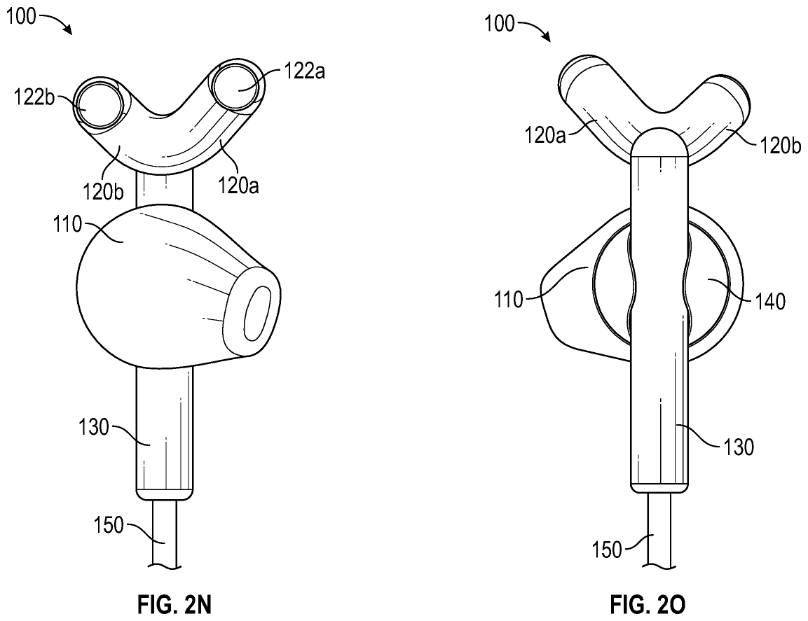

FIGS. 2A-2G illustrate various views of an example embodiment of an auricular device 100. Auricular device 100 can include a first portion that can secure to and/or within (for example, at least partially within) an ear canal of a user and a second portion coupled with the first portion that can be placed and/or positioned proximate and/or adjacent (e.g., next to, within or in contact with) the concha cymba of an ear of the user when the first portion is secured to and/or within the ear canal. Such first portion of auricular device 100 can be for example, an ear canal element 110. The ear canal element 110 can be sized and/or shaped to fit within (or at least partially within) an ear canal of a user. For example, the ear canal element 110 can include a narrowed and/or tapering tip that can facilitate insertion and/or securement within a portion of the ear canal. The ear canal element 110 can be similar to those adapted for use with audio microphones (e.g., earbuds) in some embodiments, for example. In some embodiments, the ear canal element 110 is a customized 3D printed component to allow for a better fit in the ear canal of a user. In some embodiments, the auricular device 100 is configured to allow various ear canal elements to be swapped and/or interchanged to provide sizing flexibility. Such second portion of auricular device 100 can be and/or include one or more prongs, such as one or both of prongs 120a, 120b. Prongs 120a, 120b can include and/or operably position one or more electrodes. For example, auricular device 100 can include a first electrode 122a coupled to and/or positioned along prong 120a and/or can include a second electrode 122b coupled to and/or positioned along prong 120b (see FIG. 2C). The first electrode 122a can be an active electrode and the second electrode 122b can be a return electrode, or vice versa. The first electrode 122a can be coupled to an end of the prong 120a and/or the second electrode 122b can be coupled to an end of the prong 120b. In some embodiments, the auricular device 100 does not include a hydrogel material adjacent and/or on the first electrode 122a and/or does not include a hydrogel material adjacent and/or on the second electrode 122b. Additionally or alternatively, in some embodiments, the auricular device 100 does not include an adhesive material adjacent and/or on the first electrode 122a and/or does not include an adhesive material adjacent and/or on the second electrode 122b.

The first and second portions of the auricular device 100 discussed above can be coupled to one another. For example, the auricular device 100 can include a third portion that can couple the first and second portions together. Such third portion can be, for example, a stem 130 and/or a boss 140 (discussed below). Stem 130 can be an elongated element (for example, stem 130 can have a length or height that is greater than one or more dimensions of a cross-section of the stem 130). In some embodiments, auricular device 100 includes a boss 140 (which can also be referred to herein as a “coupler” or “adapter”) that can couple the first and/or second portions of the auricular device 100 to one another, for example, along with the stem 130. For example, auricular device 100 can include a boss 140 that can couple (for example, directly or indirectly couple) the ear canal element 110 to the stem 130, and, therefore, can couple the ear canal element 110 to the prong(s) 120a, 120b. In some embodiments, the boss 140 can removably couple to the stem 130. For example, in some embodiments, the boss 140 can be configured to secure to the stem 130 via a snap-fit arrangement.

In some embodiments, the auricular device 100 includes a cable 150 that can facilitate electrical connection between electrical components of the auricular device 100 (for example, electrodes 122a, 122b of the auricular device 100) to a power source. Such power source can be spaced from and/or separate from the auricular device 100. For example, the auricular device 100 can include a cable 150 that connects to a power source integrated into a housing or enclosure that is attached to a portion of the user (for example, secured behind the ear, over the ear, in a headband secured around the user's head, around a neck of the user, and/or around an arm of the user). As another example, the auricular device 100 can include a cable 150 that connects to a power source integrated into a housing or enclosure that is attached to a user's (e.g., patient) upper arm, which could also contain a blood pressure cuff that can be used as a therapy sensor. In some embodiments, the auricular device 100 is configured to receive power via cable 150, which can also be configured to facilitate audio delivery via ear canal element 110. In some embodiments, cable 150 is a 2.5 mm cable.

In some embodiments, the auricular device 100 does not include a cable (such as cable 150). For example, in some embodiments, the auricular device 100 includes a power source to provide power to electrical components of the auricular device 100 (for example, electrodes 122a, 122b of the auricular device 100). For example, any of the ear canal element 110, stem 130, boss 140, and/or prongs 120a, 120b can include a power source (e.g., a battery) that can provide power to electrical components of the auricular device 100 (for example, electrodes 122a, 122b of the auricular device 100).

In some embodiments, the auricular device 100 includes one or more sensors for calibration or for the purpose of therapy delivery (e.g., closed-loop therapy delivery), including but not limited to, a photoplethysmography sensor (PPG), a galvanic skin sensor (GSR), an inertial measurement unit sensor (IMU), a temperature sensor, a respiratory sensor, and an Electroencephalography sensor (EEG). In some embodiments, respiration is measured with mechanical, electrical, impedance, acoustic (e.g., microphone), ultrasonic, infrared, or video based measures. Alternatively or additionally, any of such above-described sensors can be incorporated into the housing or enclosure that is separate and/or spaced from the auricular device 100 such as that discussed above.

As discussed above and with continued reference to FIGS. 2A-2G, the stem 130 can have a generally elongated shape. Stem 130 can include a cylindrical shape, among others. Stem 130 can include a circular cross-section, for example. Stem 130 can include a first end that connects to and/or receives a portion of cable 150 (for example, where auricular device 100 includes such cable 150) and stem 130 can include a second end that connects to prongs 120a, 120b. In some embodiments, stem 130 comprises a hollow interior sized and/or shaped to receive cable 150.

As discussed above, auricular device 100 can include one or more prongs, such as prongs 120a, 120b. In some embodiments, auricular device 100 includes both of prongs 120a, 120b. Alternatively, in some embodiments, auricular device 100 includes only one of prong 120a or prong 120b. Prong 120a and/or prong 120b can extend from a portion of stem 130. For example, prong 120a and/or prong 120b can extend from an end of stem 130. Prong 120a and/or prong 120b can extend from stem 130 away from one another. For example, each of the prongs 120a, 120b can have a first end connected to the stem 130 and a second end (which can be referred to as a “free” end) that is opposite the first end. The prongs 120a, 120b can extend from stem 130 such that such second or free ends of the prongs 120a, 120b are spaced away from one another. Prongs 120a, 120b can comprise a cylindrical shape, among others. Prongs 120a, 120b can include a hollow interior sized and/or shaped to receive the cable 150 a portion thereof, or a cable coupled to cable 150, for example, where cable 150 electrically connects electrodes 122a, 122b to a power source. Prongs 120a, 120b can have a circular cross-section, for example, among others.

Auricular device 100 can include one or more electrodes positioned at or near the free ends of the prongs 120a, 120b. For example, auricular device 100 can include an active electrode 122a at a free end of the prong 120a and a return electrode 122b at the free end of the prong 120b. In some embodiments, the free ends of the prongs 120a, 120b and thus the electrodes 122a, 122b are positioned a certain distance from one another, for example, between approximately 5 mm and approximately 20 mm from one another. For example, the free ends of the prongs 120a, 120b and thus the electrodes 122a, 122b can be positioned between approximately 6 mm and approximately 19 mm, between approximately 7 mm and approximately 18 mm, between approximately 8 mm and approximately 17 mm, between approximately 9 mm and approximately 16 mm, between approximately 10 mm and approximately 15 mm, between approximately 11 mm and approximately 14 mm, between approximately 12 mm and approximately 13 mm, or between approximately 11 mm and approximately 12 mm, or any value therebetween, or any range bounded by any combination of these values, although values outside these values or ranges can be used in some cases. As another example, in some embodiments, the free ends of the prongs 120a, 120b and thus the electrodes 122a, 122b are positioned approximately 11.5 mm from one another. Such configurations can advantageously maximize vagal nerve stimulation and can accommodate a high variations in ear anatomy and/or characteristics.

With reference to at least FIGS. 2B-2C and 2F, the prongs 120a, 120b can be angled with respect to one another at an angle that is between approximately 5° and approximately 120°. For example, prongs 120a, 120b can be angled with respect to one another at an angle that is between approximately 10° and approximately 110°, between approximately 20° and approximately 100°, between approximately 30° and approximately 90°, between approximately 40° and approximately 80°, between approximately 50° and approximately 70°, between approximately 30° and approximately 90°, between approximately 40° and approximately 80°, between approximately 40° and approximately 70°, between approximately 40° and approximately 60°, or between approximately 40° and approximately 50°, or any value therebetween, or any range bounded by any combination of these values, although values outside these values or ranges can be used in some cases.

In some embodiments, a diameter of an electrode-skin contact surface area of each of the electrodes 122a, 122b is between approximately 1 mm and approximately 10 mm, for example, between approximately 2 mm and approximately 9 mm, between approximately 3 mm and approximately 8 mm, between approximately 4 mm and approximately 7 mm, between approximately 5 mm and approximately 6 mm, between approximately 3 mm and approximately 5 mm, or between approximately 2 mm and approximately 6 mm, or any value therebetween, or any range bounded by any combination of these values, although values outside these values or ranges can be used in some cases. As another example, in some embodiments a diameter of an electrode-skin contact surface area of each of the electrodes 122a, 122b is approximately 4 mm. Such configurations can advantageously maximize vagal nerve stimulation and can accommodate a high variations in ear anatomy and/or characteristics.

In some embodiments, when the auricular device 100 is in use (for example, when the ear canal element 110 is at least partially secured within an ear canal of a user), the prong(s) 120a, 120b are configured to exert a normal force into and/or on the concha cymba that is between approximately 0.1 N and approximately 1 N. For example, such normal force exerted can be between approximately 0.2 mm and approximately 0.9 mm, between approximately 0.3 mm and approximately 0.8 mm, between approximately 0.4 mm and approximately 0.7 mm, or between approximately 0.5 mm and approximately 0.6 mm, or any range bounded by any combination of these values, although values outside these values or ranges can be used in some cases. In several embodiments, exertion of a normal force at a helix, scapha, anti-helix, triangular fossa, superior crus, inferior crus, helicis crus, tragus, intertragic notch, lobule, anti-tragus, concha cavum, and/or concha cymba, or combinations thereof are provided.

In some embodiments, the auricular device 100 can be configured to allow the ear canal element 110 to move relative to the stem 130, which can in turn allow the ear canal element 110 and the prongs 120a, 120b (and/or electrodes 122a, 122b) to move relative to one another. Additionally or alternatively, in some embodiments, the auricular device 100 can be configured to allow the ear canal element 110 to rotate relative to the stem 130 and/or prongs 120a, 120b, and/or electrodes 122a, 122b. For example, the auricular device 100 can include boss 140 which can facilitate such movement and/or rotation.

FIGS. 2H-2J illustrate various views of the boss 140 and the ear canal element 110 without also showing other components of the auricular device 100. As shown, the boss 140 can be coupled to the ear canal element 110. The boss 140 can be rotatably coupled to the ear canal element 110 to allow the boss 140 and ear canal element 110 to rotate with respect to one another. For example, the boss 140 and/or the ear canal element 110 can be coupled to one another to facilitate 360° rotation relative to one another, or an amount or range less than 360°. Boss 140 can include a pocket 142 sized and/or shaped to receive and/or secure the stem 130 or a portion thereof. The pocket 142 can have a circular or partially circular cross-section, for example (see FIG. 2). The pocket 142 can be sized and/or shaped to surround all or a portion of a perimeter of a cross-section of the stem 130. For example, the pocket 142 can be sized and/or shaped to surround less than an entirety of a perimeter of a cross-section of the stem 130. Such configuration can allow the stem 130 to be inserted into the pocket 142 transverse (e.g., perpendicular) to an axis extending through the pocket 142 and/or parallel to such axis. The pocket 142 can allow the boss 140 and stem 130 to move relative to one another (for example, linearly or longitudinally) while the boss 140 and stem 130 are coupled to one another. Because the boss 140 and stem 130 can be coupled to the ear canal element 110 and prongs 120a, 120b, such relative movement between the boss 140 and stem 130 can therefore allow the prongs 120a, 120b and the ear canal element 110 to move relative to one another. Such configuration advantageously allows the auricular device 100 to be adjusted to accommodate anatomy of the user, such as variable distances between the user's ear canal and the concha cymba.

In some embodiments, the auricular device 100 includes a mechanism to allow the boss 140 and the stem 130 to be held or removably fixed at certain positions. For example, boss 140 can include a protrusion 144 that can interact with one or more notches 132 of the stem 130 to facilitate adjustment of the boss 140 and stem 130 in various positions. Protrusion 144 can be positioned within the pocket 142 in some embodiments. For example, in some embodiments the protrusion 144 is located at or near a center of the pocket 142 and/or extends outward from a surface of the pocket 142. Protrusion 144 can be rounded, for example, can have a half-circle or arch shape. Notches 132 can be recessed from an exterior surface of the stem 130 and can be sized and/or shaped to receive all or a portion of the protrusion 144. The stem 130 can include one, two, three, four, five, six, seven, or eight or more notches 132, and such notches 132 can be equally or non-equally spaced from another along a length of the stem 130. The securement (for example, removable securement) of the protrusion 144 within the one or more notches 132 can be a snap-fit or other type of securement, for example. The protrusion 144 can have a rounded and/or curved structure and/or shape to facilitate smooth transition into and/or out of the notches 132. In some embodiments, the ear canal element 110 and/or the boss 140 (discussed elsewhere herein) can be disposable and the stem 130, prongs 120a, 120b, and/or cable 150 are reusable.

In some embodiments, stem 130 is straight (for example, not curved). However, in alternative embodiments, the stem 130 is curved. In some embodiments, the stem 130 is rigid. Alternatively, in some embodiments, the stem 130 is flexible.

The above-described features that can allow the boss 140 and stem 130 (and in turn, the ear canal element 110 and the prongs 120a, 120b, electrodes 122a, 122b) to move and/or rotate relative to one another advantageously allows the auricular device 100 to provide a “one-size-fits-all” solution to accommodate a high variation in ear anatomies or characteristics when providing electrical neuromodulation (e.g., stimulation) to the vagal nerve via the concha cymba.

The stem 130, boss 140, prongs 120a, 120b, and/or ear canal element 110 can comprise, in part or in whole, plastic. Alternatively or additionally, the stem 130, boss 140, prongs 120a, 120b, and/or ear canal element 110 can comprise, in part or in whole, silicone, silicone-like material, such as fluorosilicone or other elastomer.

As discussed elsewhere herein, auricular device 100 can include prongs 120a, 120b which each can include and/or operably position an electrode at or proximate the concha cymba of a subject's ear. In some implementations, one or both of such prongs 120a, 120b are flexible and/or resilient. One or both of such prongs 120a, 120b can be independently flexible, for example, with respect to each other and/or other portions of the auricular device 100 (e.g., the ear canal element 110 and/or stem 130). One or both of such prongs 120a, 120b can be configured to allow each of the active and return electrodes 122a, 122b (discussed herein) to simultaneously make independent contact with portions of the concha cymba. One or both of such prongs 120a, 120b can be configured to allow each of the active and return electrodes 122a, 122b (discussed herein) to simultaneously exert a force (e.g., normal force) on portions of the concha cymba of between approximately 0.01 N and approximately 1 N or any of the other values for force discussed elsewhere herein, for example. Such normal force(s) can be exerted when the ear canal element 110 is secured within the ear canal of the user, for example. Such configurations can advantageously allow the prongs 120a, 120b to provide independent suspension (e.g., when engaging a subject's ear along with the ear canal element 110) that can allow each electrode coupled to the prongs 120a, 120b to independently make contact with the concha cymba in a comfortable manner without applying too much force and/or pressure. Accordingly, such configurations can reduce or eliminate the potential that contact between an electrode coupled to prong 120a and the concha cymba disrupts contact between an electrode coupled to prong 120b and the concha cymba. Any of the prongs of any of the other auricular devices discussed herein can be flexible and/or resilient as discussed above with respect to prongs 120a, 120b.

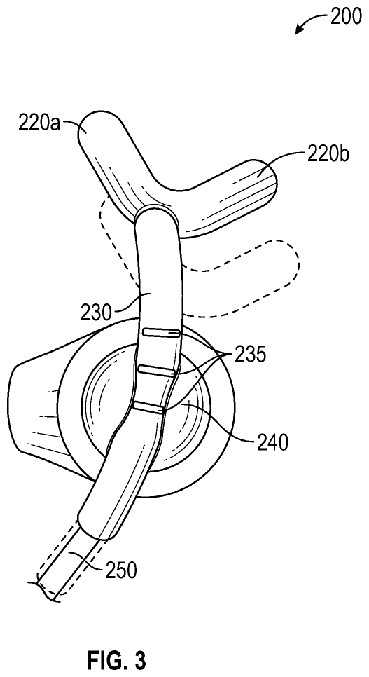

FIG. 3 illustrates another embodiment of an auricular device 200. Auricular device 200 can be the same as auricular device 100 in some or many respects. For example, the auricular device 200 can include an ear canal element 210, stem 230, boss 240, prong(s) 220a, 220b, and/or cable 250, each of which can be similar or identical to ear canal element 110, stem 130, boss 140, prong(s) 120a, 120b, and/or cable 150 discussed above with reference to auricular device 100. Prongs 220a, 220b can include electrodes similar or identical to electrodes 122a, 122b discussed above, for example, located at free ends of prongs 220a, 220b.

As illustrated in FIG. 3, stem 230 can include one or more indicators 235, each of which can be aligned and/or associated with one or more notches which can be located on another portion of the stem 230 (for example, on an opposite side or portion of stem 230). Such notches can be identical to notches 132 discussed above with respect to stem 130. Indicators 235 can advantageously indicate to a user where respective notches of the stem 230 are so as to aid the user in adjusting the distance and/or location of the boss 240 and/or ear canal element 210 relative to the stem 230, prongs 220a, 220b, and/or electrodes coupled with prongs 220a, 220b. Stem 230 can include one, two, three, four, five, six, seven, or eight or more indicators 235, and such indicators 235 can be equally or non-equally spaced from another along a length of the stem 230. Such indicators 235 can be, for example, lines extending across the surface of the stem 230.

As shown in FIG. 3, stem 230 can be curved, for example, curved between opposing ends of the stem 230. However, in alternative embodiments, the stem 230 is straight (for example, not curved). In some embodiments, the stem 230 is rigid. Alternatively, in some embodiments, the stem 230 is flexible. The stem 230, boss 240, prongs 220a, 220b, and/or ear canal element 210 can comprise, in part or in whole, plastic. Alternatively or additionally, the stem 230, boss 240, prongs 220a, 220b, and/or ear canal element 210 can comprise, in part or in whole, silicone. In some embodiments, the stem 230 comprises silicone and the boss 240 comprises plastic (for example, hard plastic).

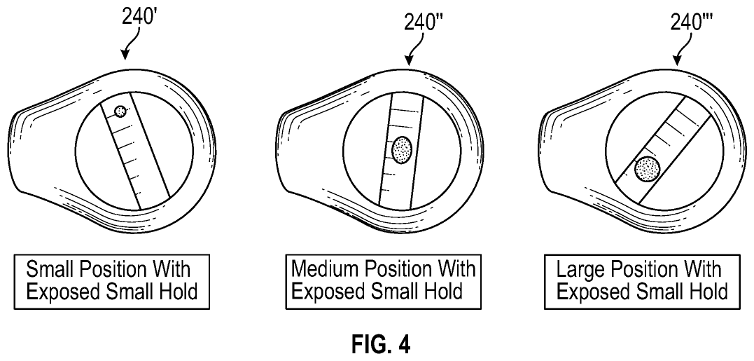

FIG. 4 illustrates bosses 240′, 240″, 240′″ that can be associated with three different sizings and/or settings of auricular device 200 (or any of the auricular devices discussed herein), where each sizing includes a locking position of stem 230 such that rotation and axial/linear movement of stem 230 is fixed. For example, each of such three sizings can be associated with a fixed rotation and axial/linear position of the stem 230. Such configurations can ensure that stems of the auricular devices are rotated and/or extended to a correct or optimal angle relative to the ear canal elements, for example.

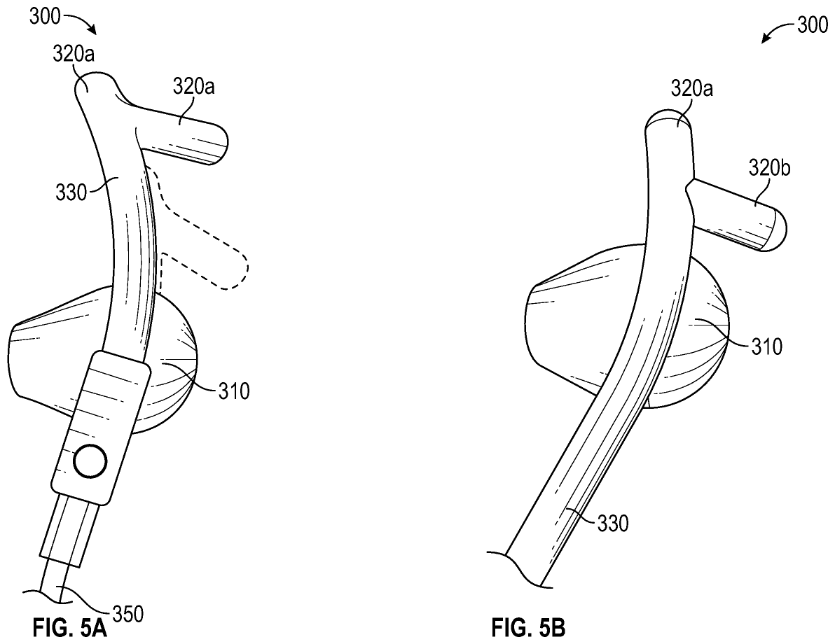

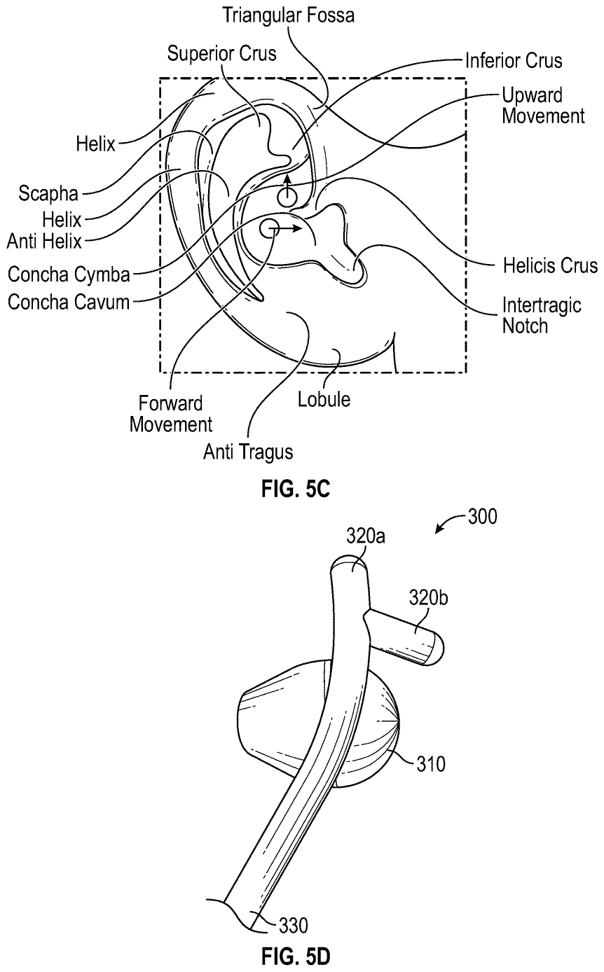

FIGS. 5A-5B illustrate another embodiment of an auricular device 300. Auricular device 300 can be the same as auricular device 100 (and/or other auricular devices discussed herein) in some or many respects. For example, the auricular device 300 can include an ear canal element 310, stem 330, prong(s) 320a, 320b, each of which can be similar or identical to ear canal element 110, stem 130, and/or prong(s) 120a, 120b, discussed above with reference to auricular device 100. Similar to stem 230, stem 330 can be curved, for example, along all or a portion of its length (for example, less than half of its length). Prongs 320a, 320b can extend from stem 330 and can be angled, for example, at an angle such as any of those discussed above with respect to prongs 120a, 120b. In one embodiment, prongs 320a, 320b are angled approximately 90° (e.g., 70°-80°, 80°-90°, 90°-100°, and overlapping ranges therein). relative to one another. Prong 320a can form and/or be part of an extension of the stem 330, for example, can extend along a same path of the stem 330.

With reference to FIGS. 5C and 5D, stem 330 can be a curved, flexible element that can be moved linearly and/or longitudinally relative to the ear canal element 310 to move or position the prongs 320a, 320b in an upward and/or forward position proximate the concha cymba. In some embodiments, the ear canal element 310 can be manufactured separately from the stem 330 and/or prongs 320a, 320b.

FIGS. 6A-6B illustrate another embodiment of an auricular device 400. Auricular device 400 can include an ear canal element 410 that can secure to and/or within (for example, at least partially within) an ear canal of a user, a wire element 430, and a boss 440 that can couple the ear canal element 410 to the wire element 430. The boss 440 can include one or more openings (for example, holes) sized and/or shaped to receive the wire element 430 and configured to allow the boss 440 to move relative to the wire element 430, for example, along a length of the wire element 430. Such configuration can in turn allow the ear canal element 410 to move relative to the wire element 430 when the boss 440 is coupled with the ear canal element 410. The wire element 430 can include one or more electrodes, such as electrodes 422a, 422b. Electrodes 422a, 422b can be active and return electrodes. Electrodes 422a, 422b can be located along a portion of the wire element 430 that is defined and/or partitioned by the coupling with the boss 440 and/or openings (e.g., holes) in the boss 440 that receive the wire element 440. The length and/or size of such defined and/or partitioned portion of the wire element 430 that includes electrodes 422a, 422b can be adjusted, for example, by movement of the boss 440 along the wire element 430. Such configurations can advantageously allow the defined and/or partitioned portion of the wire element 430 to be adjusted to a size and/or shape of a concha cymba of a given user. In some embodiments, one or more clips or other attachments/coupling devices are used to couple the neuromodulation device to the ear. In some embodiments, an earbud is used. Vibrational therapy is included in some embodiments.

Electrodes 422a, 422b can be pre-mounted or over-molded to the wire element 430. Electrodes 422a, 422b can be fixed on the wire element 430 or can be configured to be moved along the wire element 430. Electrodes 422a, 422b can be spaced from one another along the wire element 430 at a distance 425 that can be identical to the distances discussed above with respect to electrodes 122a, 122b (e.g., at approximately 10-14 mm, such as 11.5 mm).

FIG. 7 illustrates another embodiment of an auricular device 400′ that can be identical to auricular device 400 except that it includes placement indicators 480′ and a separate component 470′ that includes electrodes 422a, 422b having a pre-defined spacing (for example, the distances discussed above with respect to electrodes 122a, 122b) which can be coupled to the wire element 430 in accordance with indicators 480′. Indicators 480′ can be printed on the wire element 430 for example. Alignment of a portion of the separate component 470′ with one or more of the placement indicators 480′ can facilitate a small, medium, or large “sizing” for users. Such separate component 470′ can be adjustable by a user or locked in place and/or can be pre-threaded onto the wire element 430 or configured to clip onto the wire element 430.

### Neuromodulation Device

FIG. 8A illustrates a block diagram of an example neuromodulation (e.g., neurostimulation) device 800. In several embodiments, the features discussed with reference to neuromodulation device 800 can form part of and/or be incorporated into any of the auricular devices described herein that can be placed in or adjacent to the ear of a user. However, the features discussed with reference to neuromodulation device 800 are not so limited, and can be incorporated into other types of neuromodulation devices. The device 800 includes multiple hardware components which are capable of, or programmed to provide therapy across the skin of the user. As illustrated in FIG. 8A, some of these hardware components may be optional as indicated by dashed blocks. In some instances, the device 800 may only include the hardware components that are required for stimulation therapy. The hardware components are described in more detail below.

The device 800 can include two or more effectors, e.g. electrodes 802 for providing neurostimulation signals. In some instances, the device 800 is configured for transcutaneous use only and does not include any percutaneous or implantable components. In some embodiments, the electrodes can be dry electrodes. In some embodiments, water or gel can be applied to the dry electrode or skin to improve conductance. In some embodiments, the electrodes do not include any hydrogel material, adhesive, or the like. In one embodiment, one or more implantable component(s) are provided.

The device 800 can further include stimulation circuitry 804 for generating signals that are applied through the electrode(s) 802. The signals can vary in frequency, phase, timing, amplitude, or offsets. The device 800 can also include power electronics 806 for providing power to the hardware components. For example, the power electronics 806 can include a battery.

The device 800 can include one or more hardware processors 808. The hardware processors 108 can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein. In an embodiment, all of the processing discussed herein is performed by the hardware processor(s) 808. The memory 810 can store data specific to patient and rules of operation as discussed below.

In the illustrated figure, the device 800 can include one or more sensors 812. As shown in the figure, the sensor(s) 812 may be optional. Sensors could include, for example, biomechanical sensors configured to, for example, measure motion, respiration, and/or bioelectrical sensors (e.g., EMG, EEG, and/or nerve conduction sensors). Sensors can include, for example, cardiac activity sensors (e.g., ECG, PPG), skin conductance sensors (e.g., galvanic skin response, electrodermal activity), respiration sensors (e.g., respiratory effort belt, acoustic, microphone), and motion sensors (e.g., accelerometers, gyroscopes), and combinations thereof. The one or more sensors 102 may include an inertial measurement unit (IMU).

In some embodiments, the IMU can include one or more of a gyroscope, accelerometer, and magnetometer. The IMU can be affixed or integrated with the neuromodulation (e.g., neurostimulation) device 800. In an embodiment, the IMU is an off the shelf component. In addition to its ordinary meaning, the IMU can also include specific components as discussed below. For example, the IMU can include one more sensors capable of collecting motion data. In an embodiment, the IMU includes an accelerometer. In some embodiments, the IMU can include multiple accelerometers to determine motion in multiple axes. Furthermore, the IMU can also include one or more gyroscopes and/or magnetometer in additional embodiments. Since the IMU can be integrated with the neurostimulation device 800, the IMU can generate data from its sensors responsive to motion, movement, or vibration felt by the device 800. Furthermore, when the device 800 with the integrated IMU is worn by a user, the IMU can enable detection of voluntary and/or involuntary motion of the user.

The device 800 can optionally include user interface components, such as a feedback generator 814 and a display 816. The display 816 can provide instructions or information to users relating to calibration or therapy. The display 816 can also provide alerts, such an indication of response to therapy, for example. Alerts may also be provided using the feedback generator 814, which can provide haptic feedback to the user, such as upon initiation or termination of stimulation, for reminder alerts, to alert the user of a troubleshooting condition, among others. Accordingly, the user interface components, such as the feedback generator 814 and the display 816 can provide audio, visual, and haptic feedback to the user.

Furthermore, the device 800 can include communications hardware 818 for wireless or wired communication between the device 800 and an external system, such as the user interface device discussed below. The communications hardware 818 can include an antenna. The communications hardware 818 can also include an Ethernet or data bus interface for wired communications.

While the illustrated figure shows several components of the device 800, some of these components are optional and not required in all embodiments of the device 800. In some embodiments, a system can include a diagnostic device or component that does not include neuromodulation functionality. The diagnostic device could be a companion wearable device connected wirelessly through a connected cloud server, and include, for example, sensors such as cardiac activity, skin conductance, respiration, and/or motion sensors as described elsewhere herein.

In some embodiments, the device 800 can also be configured to deliver one, two or more of the following: magnetic, vibrational, mechanical, thermal, ultrasonic, or other forms of stimulation instead of, or in addition to electrical stimulation. Such stimulation can be delivered via one, two, or more effectors in contact with, or proximate the skin surface of the patient. However, in some embodiments, the device is configured to only deliver electrical stimulation, and is not configured to deliver one or more of magnetic, vibrational, mechanical, thermal, ultrasonic, or other forms of stimulation.

Although several neurostimulation devices are described herein, in some embodiments nerves are modulated non-invasively to achieve neuro-inhibition. Neuro-inhibition can occur in a variety of ways, including but not limited to hyperpolarizing the neurons to inhibit action potentials and/or depleting neuron ion stores to inhibit firing action potentials. This can occur in some embodiments via generating neuro-excitation or neuro-inhibition. For example, anodal or cathodal stimulation, low frequency stimulation (e.g., less than about 5 Hz in some cases), or continuous or intermediate burst stimulation (e.g., theta burst stimulation) can be implemented. In some embodiments, the wearable devices have at least one implantable portion, which may be temporary or more long term. In many embodiments, the devices are entirely wearable and non-implantable. In some embodiments, the frequency does not exceed 1 kHz, 5 kHz or 15 kHz. In some embodiments, theta burst stimulation is applied from 1 Hz to 10 Hz (e.g., 1, 2, 3, 4, 5, 6, 7, 8, 9, 10 Hz, and overlapping ranges therein (e.g., 1-2, 2-3, 3-4, 4-5, 5-6, 6-7, 7-8, 8-9, 9-10, 1-3, 1-4, 1-5, 1-6, 1-7, 1-8, 1-9, 2-4, 2-5, 2-6, 2-7, 2-8, 2-9, 2-10, 3-5, 3-6, 3-7, 3-8, 3-9, 3-10, 4-6, 4-7, 4-8, 4-9, 4-10, 5-7, 5-8, 5-9, 5-10, 6-8, 6-9, 6-10, 7-9, 7-10, and 8-10 Hz).

Any of the auricular devices discussed herein can be used for treatment of a number of diseases and conditions including but not limited to: depression, such as depression associated with Parkinson's disease and/or postpartum; gastrointestinal issues, such as gastrointestinal issues associated with Parkinson's and/or postpartum; inflammation, such as inflammation associated with Crohn's disease, rheumatoid arthritis (RA), multiple sclerosis (MS), psoriatic arthritis, osteoarthritis, and/or psoriasis; Lyme disease; Alzheimer's disease; atrial fibrillation; migraine; addiction; stress; tinnitus, among other things. Immune dysfunction is treated in several embodiments. In several embodiments, the auricular devices described herein can be unilateral or bilateral (e.g., placed in both ears) and can be used alone or in combination with other types of neuromodulation devices.

In several embodiments, neuromodulation such as nerve stimulation can provide therapeutic benefit across a variety of diseases, including but not limited to movement disorders (including but not limited to essential tremor, Parkinson's tremor, orthostatic tremor, and multiple sclerosis), urological disorders, gastrointestinal disorders, cardiac diseases, inflammatory diseases (for example neuroinflammation), mood disorders (including but not limited to depression, bipolar disorder, dysthymia, and anxiety disorder), pain syndromes (including but not limited to migraines and other headaches, trigeminal neuralgia, fibromyalgia, complex regional pain syndrome), Lyme disease, stroke, among others. Inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory diseases are treated in several embodiments. Cardiac conditions (such as atrial fibrillation, hypertension, and stroke) are treated in one embodiment. Epilepsy and other seizure disorders are treated in one embodiment. Inflammatory skin conditions and immune dysfunction are also treated in some embodiments. Other disorders can also be treated. For example, stimulation of the vagus nerve has been shown to improve symptoms of hypertension, dexterity, and cardiac dysrhythmias.

In one embodiment, a method framework for treating rheumatoid arthritis, atrial fibrillation or migraine with peripheral nerve stimulation comprises a plurality of therapy pathways: (a) an acute relief pathway, and/or (b) a preventative therapy pathway. In one embodiment, the therapy framework and each therapy pathway may be executed by applying peripheral nerve stimulation via a wrist worn neuromodulation device, an auricular neuromodulation device, or any combination of wrist worn devices and auricular devices. One or both ears and/or wrists may be stimulated.

As used herein, a “preventative therapy pathway” or “to prevent” shall include a reduction in full blown symptoms associated with a particular disease or condition. For example, a user may activate neurostimulation at the onset of a mild symptom, thus preventing (or stopping or reducing) additional symptoms from occurring. As a non-limiting example, upon experiencing a prodromal (pre-headache) symptom, the use of neurostimulation as described herein is used to prevent other, more severe symptoms from occurring. For example, in some embodiments, the neurostimulation applied to the user as part of the preventative therapy reduces a likelihood that the user will develop symptoms that would change the treatment protocol to the acute relief pathway. In one embodiment, a preventative therapy pathway stops or reduces more severe symptoms by 50-95% (e.g. by more than 70%, 80%, 90%, etc.).

In some embodiments, disorders and symptoms caused or exacerbated by microbial infections (e.g., bacteria, viruses, fungi, and parasites) are treated. Symptoms include but are not limited to sympathetic/parasympathetic imbalance, autonomic dysfunction, inflammation (e.g., neuroinflammation), motor and balance dysfunction, pain and other neurological symptoms. Disorders include but are not limited to tetanus, meningitis, Lyme disease, urinary tract infection, mononucleosis, chronic fatigue syndrome, autoimmune disorders, etc. In some embodiments, autoimmune disorders and/or pain unrelated to microbial infection is treated, including for example, inflammation, headache, back pain, joint pain and stiffness, muscle pain and tension, etc.

Bradykinesia, dyskinesia, gait dysfunction, dystonia and/or rigidity may also be treated according to several embodiments.

The devices, systems and methods described herein are used to treat Lyme disease (e.g., its associated symptoms) in some embodiments. The inflammation associated with Lyme disease is reduced in one embodiment (including for example, long term or chronic inflammation and/or flare ups). Resulting neurological conditions are treated in some embodiments, including but not limited to, weakness, numbness, nerve damage, and facial muscle paralysis. In addition to Lyme disease, chronic fatigue syndrome and its associate symptoms, such chronic inflammation, flare ups etc. are treated according to several embodiments. Treatment may be accomplished by, for example, vagal stimulation and/or sympathetic/parasympathetic balance. In some embodiments, the vagus nerve, trigeminal nerve and/or greater auricular nerve is/are neuromodulated. In some embodiments, only the vagus nerve (e.g., auricular and/or non-auricular branches) is neuromodulated. In some embodiments, the vagus nerve (e.g., auricular and/or non-auricular branches) and one, two or more other nerves are neuromodulated (e.g., trigeminal nerve, greater auricular nerve, nerves of the auricular branch, etc.). In some embodiments, the vagus nerve (e.g., auricular and/or non-auricular branches) is not stimulated and instead, for example, one or more other nerves are stimulated (e.g., trigeminal nerve, greater auricular nerve, other nerves of the auricular branch, etc.). For example, the auricular branch of the vagus nerve may be stimulated while other portions of the vagus nerve are not stimulated. In another embodiment, the auricular branch of the vagus nerve is stimulated before, after or during stimulation non-auricular portions of the vagus nerve (or non-vagus nerves). Neuromodulation according to several embodiments includes stimulation using, for example, the parameters disclosed herein.

A second therapy (such as vibratory therapy) is provided in conjunction with neurostimulation disclosed herein in some embodiments. A second therapy may be at a lower or higher frequency than the first stimulator, and may include for example ultrasonic nerve effector(s) such as piezoelectronic elements. In some embodiments, the effector can be a phased array ultrasonic (e.g., focused ultrasound) effector. For example, a phased array ultrasonic effector may comprise a plurality of ultrasonic transducer elements. The elements may each have a width and a thickness. The thickness may be related to the width (e.g., thickness being a fraction (e.g., ½, ⅓, ¼, ⅕, 1/10, ranges between such values, etc.) or multiple (e.g., 2×, 3×, 4×, 5×, 10×, ranges between such values, etc.) of the width). The elements may each have a width and a space between the elements may be related to the width (e.g., the same as the width, half the width, twice the width). Spacing between the elements may be adjustable. In some embodiments, the elements have a width between about 0.5 mm and about 2 mm and a spacing between about 0.1 mm and about 2 mm. The elements may be arranged in a one-dimensional array or a two-dimensional array. The elements may be cuboid, rectangular, cylindrical, prismatic, pyramidal, or any appropriate shape. Ultrasonic signals may be, for example, between about 20 kHz and about 2 GHz or more (e.g., about 20 kHz, about 50 kHz, about 100 kHz, about 500 kHz, about 1 MHz, about 1.5 MHz, about 2 MHz, ranges between such values, and the like). At least one of the elements may transmit a different frequency. Each of the elements may transmit a different frequency. Each of the elements may transmit a same frequency. In some embodiments, a dosage level applied by an ultrasonic effector is between about 0 W/cm² and about 2 W/cm² (e.g., about 0 W/cm², about 0.1 W/cm², about 0.25 W/cm², about 0.5 W/cm², about 1 W/cm², about 1.5 W/cm², about 2 W/cm², ranges between such values, etc.). One, some, or all of the ultrasonic transducer elements may be diverging, focused, scattered, flat, etc. In some embodiments, transducer elements may be arranged in a way to focus energy (e.g., energy from different elements results in constructive interference) at a location below the surface of the skin that is in proximity to a target nerve or region of tissue. In one embodiment, ultrasonic therapy is used alone for auricular therapy.

Neuromodulation (e.g., neurostimulation), according to some embodiments, is used to replace pharmaceutical agents, and thus reduce undesired drug side effects. In other embodiments, neuromodulation, such as neurostimulation, is used together with (e.g., synergistically with) pharmaceutical agents to, for example, reduce the dose or duration of drug therapy, thereby reducing undesired side effects. Undesired drug side effects include for example, addiction, tolerance, dependence, GI issues, nausea, confusion, dyskinesia, altered appetite, etc. In various embodiments, neuromodulation, such as neurostimulation, is used together with (e.g., synergistically with) pharmaceutical agents for treatment of epilepsy, depression, anxiety, inflammatory conditions such as inflammatory bowel disease (such as Crohn's disease, colitis, and functional dyspepsia), rheumatoid arthritis, multiple sclerosis, psoriatic arthritis, psoriasis, chronic fatigue syndrome, and other inflammatory conditions (such as neuroinflammation and inflammatory skin conditions) are treated in several embodiments. Neuromodulation, such as neurostimulation, is used together with (e.g., synergistically with) pharmaceutical agents for treatment of cardiac conditions (such as atrial fibrillation, hypertension, and stroke) are treated in various embodiments. Seizure disorders are treated in one embodiment. Neuromodulation, such as neurostimulation, is used together with (e.g., synergistically with) pharmaceutical agents for treatment of headaches, such as migraine, are treated in other embodiments.

### User Interface Device

FIG. 8B illustrates communications between the neurostimulation device 800 and a user interface device 850 over a communication link 830. The communication link 830 can be wired or wireless. The neuromodulation (e.g., neurostimulation) device 800 is capable of communicating and receiving instructions from a user interface device 850. The user interface device 850 can include a computing device. In some embodiments, the user interface device 850 is a mobile computing device, such as a mobile phone, a smartwatch, a tablet, or a wearable computer. The user interface device 850 can also include server computing systems that are remote from the neurostimulation device. The user interface device 850 can include hardware processor(s) 852, a memory 854, display 856, and power electronics 858. In some embodiments, a user interface device 850 can also include one or more sensors, such as sensors described elsewhere herein. Furthermore, in some instances, the user interface device 850 can generate an alert responsive to device issues or a response to therapy. The alert may be received from the neurostimulation device 800.

In additional embodiments, data acquired from the one or more sensors 802 is processed by a combination of the hardware processor(s) 808 and hardware processor(s) 852. In further embodiments, data collected from one or more sensors 802 is transmitted to the user interface device 850 with little or no processing performed by the hardware processors 808. In some embodiments, the user interface device 850 can include a remote server that processes data and transmits signals back to the device 800 (e.g., via the cloud).

In some instances, the user interface device may be replaced or work in conjunction with a base station. The base station can be configured to stream movement sensor and usage data on a periodic basis, e.g., daily and charge the device.

Various embodiments of the devices and/or systems discussed herein can stimulate nerves in an outer ear of a user, including but not limited to the auricular branch of the vagus nerve, great/greater auricular nerve, auriculotemporal nerve, and/or lesser occipital nerve, among others. In some embodiments, stimulation may alternate between each nerve such that the nerves are not stimulated simultaneously. In some embodiments, all nerves (e.g., target nerves) are stimulated simultaneously. In some embodiments, stimulation is delivered to the various nerves in one of many bursting patterns. The stimulation parameters may include one, two, three or more of: on/off, time duration, intensity, pulse rate, pulse width, waveform shape, and the ramp of pulse on and off. In one embodiment the pulse rate may be from about 1 Hz to about 100 Hz, about 1 Hz to about 5000 Hz, about 1 Hz to about 500 Hz, about 5 Hz to about 50 Hz, about 50 Hz to about 300 Hz, or about 150 Hz. In some embodiments, the pulse rate may be from 1 kHz to 20 KHz. A pulse width may range from, in some cases, 50 to 500 μs (micro-seconds), such as approximately 300 μs. The intensity of the electrical stimulation may vary from 0 mA to 500 mA, and a current may be approximately 1 to 11 mA in some cases. As another example, the current may be between about 1 mA to about 5 mA. The electrical stimulation can be adjusted in different patients and with different methods of electrical stimulation. The increment of intensity adjustment may be, for example, 0.1 mA to 1.0 mA. In one embodiment the stimulation may last for approximately 10 minutes to 1 hour, such as approximately 10, 20, 30, 40, 50, or 60 minutes, or ranges including any two of the foregoing values. In some embodiments, a plurality of electrical stimuli can be delivered offset in time from each other by a predetermined fraction of multiple of a period of a measured rhythmic biological signal such as hand tremor, such as about ¼, ½, or ¾ of the period of the measured signal for example. In some embodiments, a plurality of electrical stimuli can be delivered offset in time from each other by a predetermined fraction during measured exhalation or during measured inhalation (for example, only during measured exhalation or only during measured inhalation). Further possible stimulation parameters are described, for example, in U.S. Pat. No. 9,452,287 to Rosenbluth et al., U.S. Pat. No. 9,802,041 to Wong et al., PCT Pub. No. WO 2016/201366 to Wong et al., PCT Pub. No. WO 2017/132067 to Wong et al., PCT Pub. No. WO 2017/023864 to Hamner et al., PCT Pub. No. WO 2017/053847 to Hamner et al., PCT Pub. No. WO 2018/009680 to Wong et al., and PCT Pub. No. WO 2018/039458 to Rosenbluth et al., Application PCT/US2022/074376, published as PCT Pub. No. WO 2023/015158 to Schulte et al., each of the foregoing of which are hereby incorporated by reference in their entirety into the disclosure.

### Controller

FIG. 8C illustrates a block diagram of an embodiment of a controller 880 that can be implemented with the hardware components described above with respect to FIGS. 8A-8B. The controller 880 can include multiple engines for performing the processes and functions described herein. The engines can include programmed instructions for performing processes as discussed herein for detection of input conditions and control of output conditions. The engines can be executed by the one or more hardware processors of the neuromodulation (e.g., neurostimulation) device 800 alone or in combination with the user interface device 850. The programming instructions can be stored in a memory 810. The programming instructions can be implemented in C, C++, JAVA, or any other suitable programming languages. In some embodiments, some or all of the portions of the controller 880 including the engines can be implemented in application specific circuitry such as ASICs and FPGAs. Some aspects of the functionality of the controller 880 can be executed remotely on a server (not shown) over a network. While shown as separate engines, the functionality of the engines as discussed below is not necessarily required to be separated. Accordingly, the controller 880 can be implemented with the hardware components described above with respect to FIGS. 8A-8B.

The controller 880 can include a signal collection engine 802. The signal collection engine 802 can enable acquisition of raw data from sensors embedded in the device, including but not limited to accelerometer or gyroscope data from the IMU 802. In some embodiments, the signal collection engine 802 can also perform signal preprocessing on the raw data. Signal preprocessing can include noise filtering, smoothing, averaging, and other signal preprocessing techniques to clean the raw data. In some embodiments, portions of the signals can be discarded by the signal collection engine 802.

The controller 880 can also include a feature extraction engine 804. The feature extraction engine 804 can extract relevant features from the signals collected by the signal collection engine 802. The features can be in time domain and/or frequency domain. For example, some of the features can include amplitude, bandwidth, area under the curve (e.g., power), energy in frequency bins, peak frequency, ratio between frequency bands, and the like. The features can be extracted using signal processing techniques such as Fourier transform, band pass filtering, low pass filtering, high pass filtering and the like.

The controller can further include a rule generation engine 806. The rule generation engine 806 can use the extracted features from the collected signals and determine rules that correspond to neurostimulation therapy. The rule generation engine 806 can automatically determine a correlation between specific extracted features and neurostimulation therapy outcomes. In some instances, the features are extracted from biosignals sensed by one or more sensors and/or the nerve effector (such as one, two, four or six stimulation electrodes). In some embodiments, the stimulation electrodes themselves are employed as sensing elements (e.g., for detecting electrodermal activity; or cardiac activity; or EEG) and can be placed on or proximate to a subject's ear or placed on or proximate to a different portion of the subject's body (such as the wrist, finger, portion of an arm, etc.). The one or more sensors can be selected from the group comprising or consisting essentially of a photoplethysmography sensor (PPG), a galvanic skin sensor (GSR), an inertial measurement unit sensor (IMU), a temperature sensor (e.g., for body/skin temperature or ambient temperature), respiratory sensors (e.g., acoustic, microphone, etc.), and/or an electroencephalography sensor (EEG) (or combinations of two or more thereof). In some embodiments, the features extracted from the biosignals include motion data, electrocardiogram, or plethysmograph signals. The rule generation engine 806 can determine stimulation patterns for improve therapy outcomes. Outcomes can include, for example, identifying patients who will respond to the therapy (e.g., during an initial trial fitting or calibration process) based on features of kinematic data (e.g., approximate entropy), predicting stimulation settings for a given patient (based on features of their condition) that will result in the best therapeutic effect (e.g., dose, where parameters of the dose or dosing of treatment include but are not limited to duration of stimulation, frequency and/or amplitude of the stimulation waveform, and time of day stimulation is applied), predicting patient condition severity at a given point, predicting patient response over time, examining patient medication responsiveness combined with condition severity over time, predicting response to transcutaneous or percutaneous stimulation, or other neurostimulation or neurosurgical procedures based off of condition features and severity over time, and predicting ideal time for a patient to receive transcutaneous or percutaneous stimulation, or deep brain stimulation or thalamotomy based off of condition features and severity over time, predicting patient reported therapy outcomes or user reported satisfaction using condition features assessed kinematic measurements from the device; predicting patient response to undesirable user experience using condition features assessed from kinematic measurements and patient usage logs from the device where undesirable user experiences can include but are not limited to device malfunctions and adverse events such as skin irritation or burn; predict patient response trends based on condition severity where trends can be assessed across total number of sessions, within a user, or across a population of users; predicting or classifying subtypes to predict user response based on kinematic analysis of condition features; predicting or classifying subtypes to provide guidance for individually optimized therapy parameters; predicting or classifying subtypes to optimize the future study design based on subtypes (e.g., selecting specific subtypes for a clinical study with specific design addressing therapy need for the subtype); and predict user or customer satisfaction (e.g., net promoter score) based on user response or other kinematic features from measured motion. Differing dosing schedules and/or differing stimulation parameters may reduce tolerance or habituation and/or may increase user comfort/compliance in some embodiments.

The conditions include but are not limited to tremor, such as essential tremor. In one embodiment, with respect to tremor, outcomes can include identifying patients who will respond to the therapy (e.g., during an initial trial fitting or calibration process) based on tremor features of kinematic data, predicting stimulation settings for a given patient (based on their tremor features) that will result in the best therapeutic effect (e.g., dose, where parameters of the dose or dosing of treatment include but are not limited to duration of stimulation, frequency and/or amplitude of the stimulation waveform, and time of day stimulation is applied), predicting patient tremor severity at a given point, predicting patient response over time, examining patient medication responsiveness combined with tremor severity over time, predicting response to transcutaneous or percutaneous stimulation, or other neurostimulation or neurosurgical procedures based off of tremor features and severity over time, and predicting ideal time for a patient to receive transcutaneous or percutaneous stimulation, or deep brain stimulation or thalamotomy based off of tremor features and severity over time, predicting patient reported therapy outcomes or user reported satisfaction using tremor features assessed kinematic measurements from the device; predicting patient response to undesirable user experience using tremor features assessed from kinematic measurements and patient usage logs from the device where undesirable user experiences can include but are not limited to device malfunctions and adverse events such as skin irritation or burn; predict patient response trends based on tremor severity where trends can be assessed across total number of sessions, within a user, or across a population of users; predicting or classifying subtypes of tremor to predict user response based on kinematic analysis of tremor features; predicting or classifying subtypes of tremor to provide guidance for individually optimized therapy parameters; predicting or classifying subtypes of tremor to optimize the future study design based on subtypes (e.g., selecting specific subtypes of essential tremor for a clinical study with specific design addressing therapy need for the subtype); and predict user or customer satisfaction (e.g., net promoter score) based on user response or other kinematic features from measure tremor motion. Differing dosing schedules and/or differing stimulation parameters may reduce tolerance or habituation and/or may increase user comfort/compliance in some embodiments. The neuromodulation (e.g., auricular, wrist, leg neurostimulation) device is used in several embodiments to identify patients who may be a candidate for other therapies, such as drug therapy, surgical intervention, deep brain stimulation or thalamotomy. Response and tolerance to the neurostimulation described herein are used, in some embodiments, to provide input to a predictive model that provides an assessment of the patient's likelihood to respond to implantable deep brain stimulation or other implantable or non-implantable therapies. Algorithms and sensor measurements from the devices described can be helpful to identify those patient who may be good or poor candidates for other therapies, such as deep brain stimulation, drug therapy, surgical intervention, thalamotomy. The devices described herein in some embodiments can be used to categorize subjects who use the device for 1 week-1 year or longer into the top or bottom 50%, 25%, or 10% of those that will respond to other such therapies. This may be especially helpful so that the patients who would benefit most of such therapies (such as deep brain stimulation) are able to obtain this treatment. This type of predictive diagnostic capability should lead to more personalized therapy and better health outcomes in several embodiments.

In some embodiments, the rule generation engine 886 relies on calibration instructions to determine rules between features and outcomes. The rule generation engine 886 can employ machine learning modeling along with signal processing techniques to determine rules, where machine learning modeling and signal processing techniques include but are not limited to: supervised and unsupervised algorithms for regression and classification. Specific classes of algorithms include, for example, Artificial Neural Networks (Perceptron, Back-Propagation, Convolutional Neural Networks, Recurrent Neural networks, Long Short-Term Memory Networks, Deep Belief Networks), Bayesian (Naive Bayes, Multinomial Bayes and Bayesian Networks), clustering (k-means, Expectation Maximization and Hierarchical Clustering), ensemble methods (Classification and Regression Tree variants and Boosting), instance-based (k-Nearest Neighbor, Self-Organizing Maps and Support Vector Machines), regularization (Elastic Net, Ridge Regression and Least Absolute Shrinkage Selection Operator), and dimensionality reduction (Principal Component Analysis variants, Multidimensional Scaling, Discriminant Analysis variants and Factor Analysis). In some embodiments, the controller 886 can use the rules to automatically determine outcomes. The controller 886 can also use the rules to control or change settings of the neurostimulation device, including but not limited to stimulation parameters (e.g., stimulation amplitude, frequency, patterned (e.g., burst stimulation), intervals, time of day, individual session or cumulative on time, and the like) as described below. In some instances, the rules may be hardcoded and need not be generated.

Accordingly, the rules can improve operation of the neuromodulation, e.g., neurostimulation device, and advantageously improve patient comfort. The generated rules can be saved in the memory 810 and/or memory 854. For example, the rules can be generated after calibration and stored prior to operation of the neurostimulation device 800. Accordingly, in some embodiments, a rule application engine 888 can apply the saved rules on new data collected by the IMU or physiological sensor(s) to determine outcomes or control the neuromodulation, e.g., neurostimulation device 100. For example, the rule application engine 888 can generate instructions for electrical stimulation patterns based on rules that are either generated by the rule generation engine 886 or stored in the memory.

### Stimulation Gating

In some embodiments, a sudden burst of stimulation (as shown in FIG. 9A) is applied in the ear. This is helpful, in one embodiment, to enhance patient comfort for certain patients where gated stimulation may be uncomfortable and surprising for a user. In some embodiments, the sudden burst of stimulation only occurs during one part of the respiration cycle. For example, in some embodiments, when the pulse turns on at full power, there is no ramping period to mask the intensity of stimulation sensations. Accordingly, in some embodiments, the rule application engine 886 can be programmed to generate stimulation instructions that are gradual upon every stimulation burst.

For example, the rule application engine 886 can generate a gradual burst pattern as shown in FIG. 9B. The burst pattern could start at a lower stimulation intensity and then gradually increase to a selected intensity. For example, if the selected intensity is set to 3 mA, at the beginning of each pulse, the amplitude can begin at an initial intensity, such as 0.4 mA and change by increments of 0.1 mA to reach the selected amplitude in a time period of 0.5 s. In some instances, the rule generation engine 886 can determine the initial intensity, increments, and the time period based on the learning algorithms discussed above.

In additional instances, the rule application engine 886 can generate a gradual initial burst pattern as shown in FIG. 9C. The initial pulse at the start of the stimulation could be ramped up. For example, if the selected amplitude is 3 mA, at the beginning of each pulse, the first phase can be elongated where the initial amplitude can start at 0.4 mA and changes by an increment of 0.1 mA to reach the selected amplitude over a time period of 0.5 s. In some instances the second phase may need to be increase in amplitude and/or duration to maintain charge balance over the full phase.

In some embodiments, the stimulation may ramp up by 0.05-0.8 mA (e.g., 0.05, 0.1, 0.2, 0.3, 0.4, 0.5, 0.6, 0.7, 0.8 mA, and overlapping ranges therein) over a period of 0.5-30 or more seconds (e.g., 0.5-2, 2-5, 5-10, 10-15, 15-30 seconds, and overlapping ranges therein) or the ramp up may increase by about 10-50%, 50-100% or double with each increment. The differential between the increments may be constant or different. For example, the gradual increase or ramp up may be 0.1 mA to 0.2 mA to 0.3 mA to 0.4 mA to 0.8 mA to 1.0 A to 1.6 A to 3.2 A (and higher if needed). Alternatively, the gradual increase or ramp up start at 0.5 mA and increase constantly by 0.2 mA to reach a setpoint such as 2.5 A. A ramp up may occur each time the stimulation is turned on initially or may occur during a treatment session when a user increases stimulation. A user may adjust the ramp up or it may be automated by the system. Ramp downs are included in some embodiments. In some embodiments, the gradual increase or ramping may also be used for non-burst stimulation (such as tonic stimulation).

The rule application engine 886 can also generate other gradual stimulation patterns that are variations of the patterns described above to improve comfort and reduce the impact of electrical stimulus for the user.

The devices, systems and methods described above and in the claims are used, in several embodiments to treat depression (including but not limited to post-partum depression, depression affiliated with neurological diseases, major depression, seasonal affective disorder, depressive disorders, etc.). Inflammation is also treated in some embodiments, including but not limited to inflammatory gastrointestinal disorders and skin disorders. Inflammation includes neuroinflammation in some embodiments. In one embodiment, Lyme disease and chronic fatigue syndrome are treated (including chronic inflammatory states and symptoms). Neurological diseases (such as Parkinson's and Alzheimer's) as well their associated symptoms and manifestations are treated in several embodiments (such as depression, tremor, movement disorders, etc.). In some embodiments, rheumatoid arthritis, multiple sclerosis, psoriatic arthritis, osteoarthritis, and psoriasis are treated. Cardiac conditions (such as atrial fibrillation, hypertension, and stroke) may also be treated via neuromodulation, as described in several embodiments herein. Epilepsy and other seizure disorders are treated in one embodiment. Headache disorders, such as migraine, are treated in other embodiments.

In some embodiments, the neuromodulation (e.g., stimulation) device described herein is placed on the wrist or the finger, or elsewhere on the arm instead of or in addition to the ear. One, two, three or four neuromodulation devices may be worn. For example, a device may be worn on or near the ear as well as on or near the wrist. When two or more devices are used, they may be activated separately or together (e.g., synchronized). In some embodiments, a system for applying neuromodulation to a subject includes multiple neuromodulation devices placed on or proximate to different portions of a subject's body. For example, such system can include a first neuromodulation device (such as any of the auricular devices described herein) that can be placed on or proximate to a subject's ear and a second neuromodulation device that can be placed on or proximate to a different portion of the subject's body (such as the wrist, finger, portion of an arm, etc.).

In some embodiments, a system can include a plurality of neuromodulation devices that communicate with each other wirelessly and provided a synchronized, patterned stimulation. In some embodiments, multiple neuromodulation devices may be in electrical connection with multiple electrode pairs to stimulate multiple nerves simultaneously. In one embodiment, a system can include a neuromodulation device on the wrist or other location of the arm to target a nerve of a subject (e.g., median nerve) and a neuromodulation device (such as any of the auricular devices described herein) in the ear to target the vagus nerve. In some embodiments, such neuromodulation device on the wrist or arm includes one or more electrodes at least partially encircling the wrist, a skin interface to ensure good electrical contact to the user, an electronics box or housing containing a stimulator, one or more physiological sensors, and other associated electronics such as a controller or processor for executing instructions, memory for storing instructions, a user interface which can include a display and buttons, a communications module, a battery that can be rechargeable, and optionally an inductive coil for charging the battery, and the like, and/or a band to hold all the components together and securely fasten the device around the wrist of a user. In some implementations, each neuromodulation device in the system can communicate with each other via a wired or wireless connection. Multiple neuromodulation devices can provide synchronized stimulation to the multiple nerves. Stimulation may be, for example, burst, offset, or alternating between the multiple nerves.

Modulation of the vagus nerve is accomplished with the devices described herein, according to several embodiments. In some embodiments, the devices described herein are used to stimulate the autonomic system. In some embodiments, the devices described herein are used to balance the sympathetic/parasympathetic systems.

In several embodiments, a method of treating a user and/or applying neuromodulation to the user includes receiving electroencephalogram (EEG) data relating to a user, and generating parameters of a first electrical stimulation signal (e.g., a first burst electrical stimulation signal) and/or a second burst electrical stimulation signal (e.g., second burst electrical stimulation signal) at least in part by analyzing the EEG data relating to the user. Such can be particularly advantageous in allowing for customized stimulation based upon the particular aberrant neuron oscillations that can be contributing to one or more conditions of the user (such as migraine or other headache pathology). In some embodiments, the EEG data may be recorded with a single channel, 2-channel, 4-channel, 8-channel, 16-channel, 32-channel system, or system with more than 32 channels, with one or more channels positioned over pre-determined regions of interest.

In some embodiments, a method for treating a neurological condition using neuromodulation is provided. The method can include any number of the following: positioning a first nerve effector (e.g., of a first neuromodulation device) on a skin surface proximate a median nerve of an arm or wrist of a user; positioning a second nerve effector (e.g., of the first or a second neuromodulation device) on a skin surface proximate a nerve other than the median nerve of the arm or wrist of the user; receiving data relating to the user, wherein said data is optionally EEG data; generating parameters of a first neuromodulation signal and a second neuromodulation signal, wherein generating parameters comprises analyzing the data relating to the user; delivering the first neuromodulation signal to the first nerve effector to modulate the median nerve; and delivering the second neuromodulation signal to the second nerve effector to modulate the nerve other than the median nerve (for example, the ulnar nerve or a vagal nerve of the user), thereby treating said neurological condition.

In some embodiments, a method for treating migraines using transcutaneous peripheral nerve stimulation can include any number of the following: positioning a first peripheral nerve effector (for example, of a first neuromodulation device) on a skin surface proximate a median nerve of an arm or wrist of a user; positioning a second peripheral nerve effector (for example, of the first or a second neuromodulation device) on a skin surface proximate a nerve other than the median nerve of the arm or wrist of the patient; transcutaneously delivering the first electrical stimulation signal to the first peripheral nerve effector to stimulate the median nerve; and transcutaneously delivering the second electrical stimulation signal to the second peripheral nerve effector to stimulate a nerve other than the median nerve.

In some embodiments, a neuromodulation device for treating migraines using transcutaneous peripheral nerve stimulation can include any number of the following: a first peripheral nerve effector configured to be placed on a skin surface proximate a median nerve of an arm or wrist of a user; a second peripheral nerve effector configured to be placed on a skin surface proximate a nerve other than the median nerve of the arm or wrist of the user; and a controller configured to: transcutaneously delivering the first electrical stimulation signal to the first peripheral nerve effector to stimulate the median nerve; and transcutaneously delivering the second electrical stimulation signal to the second peripheral nerve effector to stimulate a nerve other than the median nerve (such as the vagal nerve).

Respiratory gated auricular stimulation has been demonstrated to target brain networks involved in migraine and shows therapeutic promise. In some embodiments, systems and methods for providing neurostimulation of one, two, or more peripheral nerve targets that modulate vagal tone, parasympathetic outflow, vagal brainstem regions, sympathetic outflow, or sympathetically mediated brainstem regions, in which the stimulation is activated in phase with a portion of respiration by measurements of the respiratory cycle. In particular, the systems and methods can use a detecting device to detect respiration cycles over time. When a predetermined relationship or correlation between the detected activity and a threshold value, such as a match, rate of change in activity, or within a predefined range, a stimulator is instructed to provide stimulation to at least one or more peripheral nerves. The stimulation can be advantageously correlated to the detected respiration phase, such as exhalation, providing potential synergistically increased effect of the stimulation and thus improved therapeutic benefit. Any of the neuromodulation devices discussed (e.g., any of the auricular devices discussed herein) can be utilized for respiratory gated auricular stimulation. In several embodiments, the neurostimulation devices and methods disclosed herein do not use or rely on any respiratory gating. In several embodiments, the neurostimulation devices and methods disclosed herein use or rely on respiratory gating.

In several embodiments, peripheral nerve stimulation can advantageously have synergistic effects when combined with pharmacotherapy, including but not limited to anti-depressants including tricyclic antidepressants, selective serotonin reuptake inhibitors, and MAO inhibitors. The effects can include enhanced response to therapy, a lesser dose of tricyclic antidepressants, selective serotonin reuptake inhibitors, and MAO inhibitors needed to achieve the effects and thus lower adverse reactions, and the like. Combination therapy can be beneficial in some embodiments to reduce the time it takes to achieve a therapeutic effect (e.g., by at least 10%, 25, 50% or more, or overlapping ranges therein) or lengthens the therapeutic effect (by e.g., by at least 10%, 20%, 40% or more, or overlapping ranges therein), or improve the overall benefit (e.g., larger reduction in mood disorder symptom magnitude or frequency).

According to several embodiments, the neurostimulation embodiments described herein work synergistically with pharmacological agents. In light of the already sensitive and inflamed digestive system of many patients with inflammatory bowel diseases and other gastrointestinal conditions, this synergy is particularly beneficial because the patient, in one embodiment, will need an overall lower dosage of the pharmacological agent to achieve an efficacy comparable (or better) to that achieved without neurostimulation. These pharmacological agents may include, but are not limited to, anti-tumor necrosis factor (anti-TNF) drugs, Janus kinase (JAK) inhibitors, or 5-aminosalicyclic-acid derivatives (5-ASAs). This results in fewer undesired side effects in several embodiments.

According to several embodiments, the neurostimulation embodiments described herein work synergistically with pharmacological agents for rheumatoid arthritis. This synergy is particularly beneficial for rheumatoid arthritis because the patient, in one embodiment, will need an overall lower dosage of the pharmacological agent to achieve an efficacy comparable (or better) to that achieved without neurostimulation. These pharmacological agents may include, but are not limited to, conventional disease-modifying antirheumatic drugs (DMARDs), biologics and biosimilars, and JAK inhibitors. This results in fewer undesired side effects in several embodiments.

In several embodiments, stimulation regulated by one or more measured biological signals can advantageously have synergistic effects when combined with pharmacotherapy, including pharmacotherapy for mental health disorders, cardiac disorders, pain, and other diseases. The effects can include enhanced response to therapy, a lesser dose of the pharmacotherapy needed to achieve the effects and thus lower adverse reactions, and the like. This is beneficial in some embodiments to reduce the time it takes to achieve a therapeutic effect (e.g., by at least 10%, 25%, 50% or more, or overlapping ranges therein) or lengthens the therapeutic effect (by e.g., by at least 10%, 20%, 40% or more, or overlapping ranges therein), or improve the overall benefit (e.g., larger reduction in pain, blood pressure, heart rate, arrhythmia frequency, and the like).

In several embodiments, peripheral nerve stimulation can advantageously have synergistic effects when combined with pharmacotherapy, including triptans, ergots, or CGRP-inhibitors. The effects can include enhanced response to therapy, a lesser dose of triptans, ergots, or CGRP-inhibitors needed to achieve the effects and thus lower adverse reactions, and the like. Combination therapy can be beneficial in some embodiments to reduce the time it takes to achieve a therapeutic effect (e.g., by at least 10%, 25%, 50% or more, or overlapping ranges therein) or lengthens the therapeutic effect (by e.g., by at least 10%, 20%, 40% or more, or overlapping ranges therein), or improve the overall benefit (e.g., larger reduction in migraine symptom magnitude or frequency).

Any of the neuromodulation devices discussed herein (e.g., any of the auricular devices discussed herein) can be utilized to modulate (e.g., stimulate) vagal nerve of a subject alone or in combination with a one or more other nerves in the subject, for example, via a separate neuromodulation device, and such one or more other nerves can include, without limitation, median, radial, ulnar, peroneal, saphenous, tibial and/or other nerves or meridians accessible on the limbs.

In some embodiments, transcutaneous nerve neuromodulation at the arm and/or wrist (e.g., median and/or radial nerve stimulation) can advantageously inhibit sympathoexcitatory related increases in blood pressure and premotor sympathetic neural firing in the rostral ventrolateral medulla (rVLM). Neuromodulation of the median and/or radial nerves, for example, can provide more convergent input into cardiovascular premotor sympathetic neurons in the rVLM.

Also, in some embodiments, vagal nerve stimulation can modulate the trigeminal nuclei to inhibit inflammation. Thus, in several embodiments the vagal nerve is stimulated to reduce inflammation via a trigeminal pathway. In other embodiments, the trigeminal nerve is stimulated directly instead of or in addition to the vagus nerve. In some embodiments, transcutaneous nerve stimulation projects to the nucleus tractus solitarii (NTS) and spinal trigeminal nucleus (Sp5) regions to modulate trigeminal sensory complex excitability and connectivity with higher brain structures. Trigeminal sensory nuclei can be involved in neurogenic inflammation during migraine (e.g., characterized by vasodilation). In some embodiments, stimulation of the vagus nerve modulates the trigeminal sensory pathway to ameliorate migraine pathophysiology and reduce headache frequency and severity. For example, increased activation of raphe nuclei and locus coeruleus may inhibit nociceptive processing in the sensory trigeminal nucleus. Human skin is well innervated with autonomic nerves and neuromodulation (e.g., stimulation) of nerve or meridian points as disclosed herein can potentially help in treatment of migraine or other headache conditions. For example, transcutaneous nerve stimulation of afferent nerves in the periphery or distal limbs, including but not limited to median nerve, are connected by neural circuits to the arcuate nucleus of the hypothalamus. In some embodiments, the devices and methods described herein increase, decrease or otherwise balance vasodilation and vasoconstriction through neuromodulation (such as the vagus nerve, trigeminal nerve and/or other nerves surrounding the ear). For example, reduction of vasodilation is provided in several embodiments to treat or prevent migraine or other conditions that are exacerbated by vasodilation. In other embodiments, vasoconstriction is reduced in, for example, conditions in which dilation is beneficial (such as with high blood pressure and pain). In some embodiments, modulation of the blood vessel (either dilation or constriction) is used to treat tinnitus. In one embodiment, the devices and methods described herein reduce inflammation (including but not limited to inflammation post microbial infection), and the reduction in inflammation treats tinnitus.

In various embodiments, neuromodulation of one or more nerves of a subject is responsive to physiological parameters or other information (e.g., motion, location data of the subject) associated with the subject. Such physiological parameters or other information can include, without limitation, ground reaction force or foot pressure (e.g., force sensors or pressure insoles), muscle activity (e.g., EMG), cardiovascular measures (e.g., heart rate, heart rate variability (HRV), photoplethysmography (PPG), or ventricular and/or atrial dyssynchrony using electrodes to measure ECG and/or heart rhythm abnormalities), skin conductance (e.g., skin conductance response, galvanic skin response), respiratory rate, skin temperature, pupil diameter, and sleep state (e.g., awake, light sleep, deep sleep, REM). Using standard statistical analysis, machine learning, deep learning, or big data techniques, such as a logistical regression or a Naïve Bayesian classifier, such information can be analyzed to assess the subject's activity state, such as sedentary versus active, level of stress and the like, which in turn, can serve as a predictor migraine or headache attacks or other conditions.

Sympathetic and parasympathetic activity can be measured through several methods, including microneurography (MSNA), catecholamine tests, heart rate, HRV, or galvanic skin response. HRV can provide a quick and effective approximation of autonomic activity in the body. HRV can be determined by analyzing the time intervals between heartbeats, also known as RR intervals. Heart rate can be accurately captured, for example, through recording devices such as chest straps, finger sensors, or a nerve effector (such as one, two, four or six stimulation electrodes). The differences between successive RR intervals can provide a picture of one's heart health and autonomic activity. Generally speaking, healthier hearts have more variability between successive RR-intervals. This interbeat data can also be used to denote a user's sympathetic and parasympathetic activity levels. Through frequency-domain analysis, heartbeat frequencies can be separated into distinct bands. High-frequency signals (~0.15-0.4 Hz) can almost exclusively reflect parasympathetic activity, and low-frequency signals (~0.04-0.15 Hz) can represent a mixture of sympathetic and parasympathetic activity. Therefore, taking the ratio of high frequency (HF) to low frequency (LF) signals can yield an approximation of one's sympathetic tone. In some embodiments, HRV can be analyzed, for example, under time-domain, geometric domain methods in addition to frequency domain methods. In some embodiments, increased heart rate variability can signify increased parasympathetic response and/or decreased sympathetic response. Decreased heart rate variability can signify decreased parasympathetic response and/or increased sympathetic response. In some embodiments, a system can sense an increase or decrease in HRV of about or more than about 5%, 10%, 15%, 20%, 25%, 30%, 35%, 40%, 45%, 50%, 75%, 100%, or more over a baseline value (or target desired HRV value) and institute a change in one, two, or more stimulation modality parameters (e.g., frequency, width, phase, timing, amplitude, offsets, nerve target, etc.) accordingly. For example, in some embodiments, the one, two, or more stimulation modalities can be configured to modulate, such as increase or decrease stimulation modality parameters to one or more nerves (e.g., peripheral nerves) associated with the sympathetic and/or parasympathetic nervous system and/or change or modify which of the one or more nerves are targeted, and a response to therapy can be confirmed by sensing an increase or decrease in parasympathetic or sympathetic tone, including but not limited to increase or decrease in HRV, changes in high frequency content of HRV, and changes in the ratio of high frequency and low frequency content of HRV. In some embodiments, the one, two, or more stimulation modalities can be configured to modulate, such as increase or decrease in stimulation amplitude to one or more nerves (e.g., peripheral nerves) associated with the sympathetic and/or parasympathetic nervous system, and a response to therapy can be confirmed by sensing an increase or decrease in parasympathetic or sympathetic tone, including but not limited to increase or decrease in HRV, changes in high frequency content of HRV, and changes in the ratio of high frequency and low frequency content of HRV. In some embodiments, the one, two, or more stimulation modalities can be configured to modulate, such as increase or decrease in stimulation frequency to one or more nerves (e.g., peripheral nerves) associated with the sympathetic and/or parasympathetic nervous system, and a response to therapy can be confirmed by sensing an increase or decrease in parasympathetic or sympathetic tone, including but not limited to increase or decrease in HRV, changes in high frequency content of HRV, and changes in the ratio of high frequency and low frequency content of HRV. In some embodiments, the one, two, or more stimulation modalities can be configured to modulate, such as increase or decrease in pulse width to one or more nerves (e.g., peripheral nerves) associated with the sympathetic and/or parasympathetic nervous system, and a response to therapy can be confirmed by sensing an increase or decrease in parasympathetic or sympathetic tone, including but not limited to increase or decrease in HRV, changes in high frequency content of HRV, and changes in the ratio of high frequency and low frequency content of HRV. In some embodiments, the one, two, or more stimulation modalities can be configured to modulate, such as change or modify which of the one or more nerves associated with the sympathetic and/or parasympathetic nervous system are targeted (e.g., peripheral nerves) based on, for example, a sensed biomarker (e.g., heart rate, heart rate variability, heart rhythm, skin sympathetic nerve activity, electrodermal activity, body temperature as measured on the wrist, respiratory cycle, brain electrical activity, and/or cytokine levels, etc.), and a response to therapy can be confirmed by sensing an increase or decrease in parasympathetic or sympathetic tone, including but not limited to increase or decrease in HRV, changes in high frequency content of HRV, and changes in the ratio of high frequency and low frequency content of HRV. In some embodiments, the method provides multiple therapy pathways that are at least in part dependent on sensing of user biomarkers (e.g., heart rate, heart rate variability, heart rhythm, skin sympathetic nerve activity, electrodermal activity, body temperature as measured on the wrist, respiratory cycle, brain electrical activity, cytokine levels, physical activity, oxygen levels, etc.). In some embodiments, biomarkers can include patient demographics, prior medication usage, prior device therapy usage, and/or sleep cycles. In some embodiments additional data such as weather information in the patient's local address (e.g., air temperature, humidity, pressure, elevation, etc.) can be monitored to impact stimulation and/or treatment. In one embodiment, physical activity can be measured with a motion sensor. In one embodiment, oxygen levels can be measured with a pulse oximeter (e.g., via pulse oximetry). One or more biomarkers are sensed immediately prior to, during and/or after therapeutic stimulation in some embodiments. In another embodiment, biomarkers are sensed hours or days prior to or after stimulation. For example, bodily fluids may be used to determine elevated biomarkers such as cytokines or other inflammatory compounds, elevated microbes, low/high electrolytes, and stimulation is applied as a treatment to reduce such biomarkers (or raise them if they are lower than the desired range). Bodily fluids are used to determine biomarkers, such as blood, urine, saliva, sweat, tears, nasal excretions, etc. These may be measured with sensors that are separate from (e.g., independently from) or in communication with the neuromodulation components described herein. Sensors may measure a condition (such as microbial levels) or may be indicative of a symptom of a condition (such as high body temperature because of a microbial infection), and either or both may be treated by the neuromodulation parameters described herein. Sensors are used post treatment in several embodiments to confirm efficacy or alternate therapies (drugs, different or additional noninvasive or implantable neurostimulation, etc.).

In some embodiments, balance of parasympathetic and sympathetic activity can be assessed with frequency analysis of heart rate variability measured with pulsed plethysmography with an LED light source and optical sensor disposed in the device that measures fluctuations in light level due to blood flow that target one of the major blood vessels around the knee or in the arm or neck or ear in other embodiments. In some embodiments, heart rate could be measured using accelerometer-based sensors, a nerve effector (such as one, two, four or six stimulation electrodes), or with electrical-based sensors, similar to single or multiple-lead ECG monitors. In some embodiments, the stimulation electrodes themselves are employed as sensing elements (e.g., for detecting electrodermal activity; or cardiac activity; or EEG) and can be placed on or proximate to a subject's ear or placed on or proximate to a different portion of the subject's body (such as the wrist, finger, portion of an arm, etc.).

In some embodiments, stimulation of one, two, or more nerves in the upper and/or lower extremity can be combined with stimulation of the auricular branch of the vagal nerve (ABVN), such as by way of the concha (e.g., cymba or cavum) or tragus, to modulate vagal activity and restore balance of the autonomic nervous system. Some embodiments of disclosed systems, devices, and methods can stimulate solely the ABVN.

Any of the neuromodulation devices discussed herein (e.g., any of the auricular devices discussed herein) can be responsive to a number of episodes of symptoms, including unilateral throbbing cranial pain, sensory sensitivity to light, sound, and smell, nausea, and dysfunction of autonomic, cognitive, emotional, and motor systems in some cases. If more episodes occur in one day, treatment can be increased by increasing the amplitude of the stimulation, duration of the stimulation, or number of treatment sessions, for example. The number of episodes of symptoms could be detected in various ways to control the stimulation applied by a system and/or device(s). In some embodiments, the subject can enter events related to symptoms, including but not limited to unilateral throbbing cranial pain, sensory sensitivity to light, sound, and smell, nausea events on a mobile device that is configured to communicate directly and/or indirectly with the neuromodulation device.

In some embodiments, a neuromodulation device is applied to both wrists/arms and/or both ear to bilaterally stimulate the nerves in the wrist and/or arm and/or the ears. In some embodiments, the two bilateral neuromodulation devices (e.g., in both of the ears and/or on both of the wrists) can be operated simultaneously to stimulate target nerves at the same time. The stimulation parameters for each device may be the same, or may differ. The two devices may be in communication wirelessly to synchronize or offset the waveforms between to devices. In some embodiments, the two bilateral neuromodulation devices can be operated in an alternating fashion such that only one device delivers stimulation at a time. The alternating devices can alternate stimulation on an hourly, daily, weekly, or monthly basis; and the frequency of the alternation can be modified based on sensor measures.

Therapy Via an Acute Relief Pathway and/or a Preventative Therapy Pathway

In some embodiments, a method framework for treating rheumatoid arthritis, atrial fibrillation or migraine with peripheral nerve stimulation comprises a plurality of therapy pathways (e.g., two or more pathways): (a) an acute relief pathway, and/or (b) a preventative therapy pathway. In one embodiment, the therapy framework and each therapy pathway may be executed by applying peripheral nerve stimulation via a wrist worn neuromodulation device, an auricular neuromodulation device, or any combination of wrist worn devices and auricular devices.

In one embodiment, a first therapy pathway is an acute relief pathway comprising of two phases: a detection phase and a therapy delivery phase. In one embodiment, the detection phase is a phase of therapy in which this method identifies an incident of rheumatoid arthritis or an acute incident of atrial fibrillation or migraine. These acute occurrences may be detected by some sensor measuring biomarkers indicative of the condition in the body or may be self-reported by a user. After an acute episode is detected, the therapy delivery phase is initiated through the neuromodulation device 800. In one embodiment, the therapy delivery phase may be activated by a prompt on the neuromodulation device for the user to initiate therapy delivery, or it may be activated automatically by a processor 808 and controller or stimulation circuitry 804 within the neuromodulation device 800 once a certain sensor threshold is achieved. In one embodiment, this can also be determined by a machine learning algorithm, an evolutional algorithm, or some other form of artificial intelligence. The biomarkers measured might include any one or more of: heart rate, heart rate variability, heart rhythm, skin sympathetic nerve activity, electrodermal activity, body temperature as measured on the wrist or ear, the respiratory cycle, brain electrical activity, and/or cytokine levels.

In one embodiment, the threshold values for heartrate can be as low as at least 90 beats per minute. In one embodiment, the threshold values for heartrate variability can be as low as 1. However, these and any threshold values are likely to be patient-specific and will vary. In one embodiment, electrical stimulation for therapy delivery may be delivered in bursts of pulses. These bursts may range in frequency of 0-150 Hz (e.g., 1, 10, 20, 25, 40, 50, 60, 75, 90, 100, 110, 120, 125, 140, 150 Hz and values and ranges therein) and the pulses may range in frequency of 0-15 Hz (e.g., 1, 2, 4, 6, 8, 10, 12, 13, 15 Hz and values and ranges therein).

In one embodiment, as illustrated in FIG. 10, a framework involves a cardiac measurement task, an acute relief therapy, and a preventative therapy. In one embodiment, the cardiac measurement task comprises the user performing a 90 second heart rate and HRV measurement task via PPG while remaining still. These heart rate or HRV measurement tasks may range in time of 0-10 minutes (e.g., 0.5, 1.0, 1.5, 2.0, 2.5, 3.0, 3.5, 4.0, 4.5, 5.0, 5.5, 6.0, 6.5, 7.0, 7.5, 8.0, 8.5, 9.0. 9.5, 10 minutes and values and ranges therein). The tasks are performed when the user is most susceptible to atrial fibrillation onset (e.g., after waking in the morning). No vigorous physical activity is conducted for 10 or more minutes prior to measurement. A PPG sensor can optionally be integrated into the stimulator 800, base station or accessory device.

In one embodiment, the acute relief therapy activates if a heart rate is abnormally high (e.g., over 100 bpm) to consider if user is likely in atrial fibrillation. User delivers one or more 15-minute stimulation sessions, interleaved with cardiac measurement tasks, until the heart rate is normalized. These stimulation sessions may range in time of 0-120 minutes (e.g., 1.0, 5.0, 10, 15, 20, 25, 30, 35, 40, 45, 50, 55, 60, 65, 70, 75, 80, 85, 90, 95, 100, 105, 110, 115, 120 minutes and values and ranges therein). During initial usage, amplitude escalation is performed from one session to the next (between sensory and max tolerable amplitudes). In some embodiments, relatively low stimulation amplitudes are employed.

In one embodiment, the preventative therapy activates if HRV (LF:HF ratio) is abnormally high (e.g., ratio greater than or equal to 2, or a patient-specific measurement, the user may be pre-atrial fibrillation. User delivers one or more stimulation sessions, interleaved with cardiac measurement tasks, until the heart rate (LF:HF) is normalized. During initial usage, amplitude escalation is performed from one session to the next (between sensory and max tolerable amplitudes). In some embodiments, relatively high stimulation amplitudes are employed.

In one embodiment, a method senses biomarkers associated with rheumatoid arthritis, atrial fibrillation or migraine and provides electrical stimulation depending on the sensed biomarker levels. In various embodiments, the method incorporates various types of devices and sensors, including one or more of: wrist worn devices, auricular devices, other peripheral nerve stimulation devices, in-ear photoplethysmography sensors, electrocardiograms, temperature sensors, acoustic sensors (e.g., microphones), thermal cameras, infrared reflective or transmissive light monitors, in-ear electroencephalograms, or other sensors.

### Respiratory-Gated Auricular Vagal Afferent Nerve Stimulation

In various embodiments as illustrated at FIGS. 11-15C, a neuromodulation device 800 and system comprises an auricular device 100 that delivers electrical stimulation to the auricular branch of the vagus nerve. This system is comprised of an electrical stimulation pulse generator (stimulator) that delivers pulses of electrical stimulation to an earpiece and electrode, and in turn, to the desired nerve. In one embodiment, as illustrated at FIGS. 11 and 12, the auricular device 100 comprises an earpiece resembles an earbud with two electrodes protruding from the top of the boot 165 of the earbud/earpiece. In one embodiment, the electrodes can stimulate the auricular branch of the vagus nerve. In one embodiment, the earpiece also includes at least one sensor for measuring biomarkers of a user's physiological state and a controller that receives this information to adjust stimulation parameters.

In one embodiment, the system is configured to deliver stimulation continuously or in pulses at a pulse frequency that may range from 1-100 Hz. In one embodiment, the stimulator can have a round “puck-like” form factor and may be attached via a clip to a belt, shirt, or some other article of clothing. In one embodiment, the stimulator may also be integrated into the earpiece, so the earpiece resembles a hearing aid.

In one embodiment, as illustrated at FIG. 13, an additional stimulation device may be added to this system. This device may be a watch-like device that is worn on the wrist as is disclosed in PCT/US2022/074376 and is incorporated by reference in its entirety as part of the disclosure. This watch-like device delivers electrical stimulation to peripheral nerves in the wrist and contains a band with integrated dry electrodes. The electrodes in this band comprise two rows of three electrodes, where the center electrode in each row is a charge balance electrode and the electrodes on either side of the center electrode are stimulating electrodes. The device will deliver bursts of electrical stimulation from a stimulator integrated into the device. This device may include a user interface comprising an e-ink display on the face of the watch-like device. It may also include a base station to charge and house the device.

FIGS. 14A-14B illustrate neuromodulation devices that deliver electrical stimulation to the auricular branch of the vagus nerve according to embodiments of the disclosure. FIGS. 15A-15C illustrate neuromodulation devices that deliver electrical stimulation to the auricular branch of the vagus nerve according to embodiments of the disclosure. In one embodiment, various sensors 812 are incorporated into this system to measure various biomarkers. In one embodiment, one such sensor may be a photoplethysmography sensor and the biomarker measured is heart rate or heart rate variability. Another sensor may be an electrocardiogram and the biomarker heart rhythm. This EKG may be integrated into the watch-like device or into the base station or into a patch worn on a user's body. Another sensor may be a sensor for detecting the biomarker of skin sympathetic nerve activity. One of the previously mentioned sensors may also potentially detect electrodermal activity or skin temperature. Another potential sensor may be a mechanical sensor integrated into a belt worn around the chest that detects changes in the respiratory cycle. Another sensor may be a microphone that is worn in the ear to detect changes in the user's respiratory cycle. Another sensor may measure the temperature in the ear. Another sensor may be an infrared reflective light monitor integrated into the earpiece to detect changes in the user's respiratory cycle. Another potential sensor might be an electroencephalogram integrated into the earpiece to measure brain activity. Another potential sensor might measure cytokine levels in the body, and is integrated into the stimulator device, a skin patch, or a belt.

In one embodiment, the electrical stimulator device may communicates wirelessly to the belt respiration sensor described previously and deliver electrical stimulation to the earpiece via a conduit (wire). In this system, the earpiece may be a silicone boot 165 that rests at the entrance of the ear canal or it may be a clip that attaches to the helix of the ear and comprises a reflective or transmissive photoplethysmography sensor.

In one embodiment, the system may be used to provide acute therapy for users suffering from migraine, colitis, irritable bowel disease, rheumatoid arthritis, hypertension, an episode of atrial fibrillation, or other cardiac arrhythmias or pathologies. It may also be used to prevent future episodes of atrial fibrillation, or episodes of other cardiac arrhythmias. In one embodiment, the device comprises an earpiece containing electrodes, an electrical pulse generator, and at least one sensor. The pulse generator delivers electrical pulses to the electrodes to stimulate the auricular branch of the vagus nerve. Stimulating this nerve can deliver various therapeutic benefits, including treatment of atrial fibrillation or other cardiac arrhythmias, colitis, rheumatoid arthritis, migraine, irritable bowel disease, hypertension. This invention may be used in tandem with other neuromodulation devices (for example, those worn on the wrist) to enhance therapeutic benefits.

### Algorithms for Detecting Inspiration and Expiration Phases of Respiration

In various embodiments, an algorithm for determining a person's current respiratory phase and when a respiratory phase begins or ends is illustrated at FIGS. 16-17. In various embodiments, algorithms can be used in various applications, including, but not limited to: respiratory-gated peripheral nerve stimulation, underwater breathing apparatuses, aeronautics training, athletic training, sleep apnea, diagnosis of irregular breathing conditions, meditation, and treatment of anxiety conditions.

In one embodiment, a method of determining the respiratory phase of a user includes the use of a sensor for detecting and measuring respiration. This sensor produces some quantitative measure relating to the respiration state of the user. A controller then receives this value from the sensor and applies an algorithm that uses various parameters to determine whether a person is inhaling or exhaling.

In one embodiment, one such parameter that the algorithm uses is a respiration threshold. This threshold value is the minimum difference in amplitude between two sample values obtained from the sensor.

In one embodiment, a second potential type of parameter may be a sample check count. This sample check count is the minimum number of samples in a row required to be checked in order to consider whether an user has switched from one respiratory phase to another (e.g. inhalation to exhalation).

In one embodiment, a third potential type of parameter might be the respiration slope threshold. The respiration slope threshold is the minimum slope value to assign a change from one respiratory phase to another (e.g. inhalation to exhalation).

In one embodiment, a fourth potential parameter could be a lockout length. The lockout length is a minimum amount of time in which the algorithm is paused. The algorithm might also include a genetic evolutionary algorithm, a machine learning algorithm, or some other algorithm based in artificial intelligence. In these cases, the algorithms might rely on as few as zero parameters to determine the respiratory phase of a user.

In one embodiment, a respiratory state is determined from respiration data via a respiration threshold. The amplitude distance between samples is measure to consider a change in states. Optionally adaptive or constant respiration is considered.

In one embodiment, a respiratory state is determined from respiration data via a sample check count. The number of samples in a row, increasing or decreasing by a response threshold that are used to trigger a respiration state change.

In one embodiment, a respiratory state is determined from respiration data via a respiration slope threshold. The absolute amplitude change between the first and last samples of the sample check count in order to trigger a respiration state change.

In one embodiment, a respiratory state is determined from respiration data via a lockout length. The number of samples after a respiration state change before another respiration stated change can occur.

FIG. 17 illustrates a flow chart diagram of an embodiment of this algorithm. In various embodiments, a method of determining the respiratory phase of a user includes a sensor for detecting and measuring respiration, producing a sample value; and a controller that receives a value from the sensor and applies an algorithm that uses various parameters to determine whether a person is exhaling or inhaling.

In one embodiment, one of the parameters used by the algorithm is a respiration threshold, wherein the respiration threshold is the minimum difference in amplitude between two sample values. In one embodiment, one of the parameters used by the algorithm is a sample check count, wherein the sample check count is the minimum number of samples in a row required to be checked in order to consider whether a user has switched from one respiratory phase to another. In one embodiment, one of the parameters used by the algorithm is a respiration slope threshold, wherein the respiration slope threshold is the minimum slope value required to assign a change from one respiratory phase to another. In one embodiment, one of the parameters used by the algorithm is a lockout length, wherein the lockout length is a minimum amount of time in which the algorithm is paused. In one embodiment, the one respiratory phase to another is an inhalation to an exhalation.

### Additional Considerations and Terminology

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree. The terms “approximately”, “about”, “generally,” and “substantially” include the number after such term. For example, “about 10 mm” includes support for the value 10 mm.

Although certain embodiments and examples have been described herein, it will be understood by those skilled in the art that many aspects of the systems and devices shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (for example, not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. A processor device can include an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1.-102. (canceled)

103. A neuromodulation system comprising:
an electrical stimulation pulse generator that delivers electrical stimulation pulses to an earpiece that is configured to be placed inside an ear, the earpiece comprising two electrodes protruding from a top of a boot that rest on a concha of the ear to stimulate an auricular branch of a nerve;
one or more sensors that measure data from one or more biomarkers of a user's physiological state; and
a controller configured to receive and use the measured data to adjust one or more stimulation parameters of the electrical stimulation pulses.

104. The neuromodulation system of claim 103, wherein one of the one or more sensors is a microphone that is worn in the ear to detect changes in the user's respiratory cycle.

105. The neuromodulation system of claim 103, wherein one of the one or more sensors is an electroencephalogram sensor integrated into the earpiece to measure brain activity.

106. The neuromodulation system of claim 103, wherein one of the one or more sensors is configured for detecting at least one of skin sympathetic nerve activity, electrodermal activity, or skin temperature.

107. The neuromodulation system of claim 103, wherein one of one or more sensors is configured for measuring cytokine levels.

108. The neuromodulation system of claim 103, wherein the electrical stimulation pulse generator communicates wirelessly to a belt respiration sensor and delivers the electrical stimulation pulses to the earpiece via a conduit.

109. The neuromodulation system of claim 103, wherein:
the one or more sensors comprises a sensor configured for detecting and measuring a quantitative value generally relating to a respiratory phase of the user, wherein the quantitative value is one or more of a respiration threshold, a sample check count, a respiration slope threshold, and a lockout length; and
the controller is configured to:
apply an algorithm to the quantitative value; and
determine the respiratory phase of the user based on an application of the algorithm to the quantitative value,
wherein the determined respiratory phase is an inhalation phase or an exhalation phase.

110. The neuromodulation system of claim 109, wherein:
the quantitative value is the respiration threshold, and wherein the respiration threshold is a minimum difference in amplitude between two sample values; or
the quantitative value is the sample check count, and wherein the sample check count is a minimum number of samples in a row required to be checked in order to consider whether the user has switched from one respiratory phase to another; or
the quantitative value is the respiration slope, and wherein the respiration slope threshold is a minimum slope value required to assign a change from one respiratory phase to another; or
the quantitative value is the lockout length, and wherein the lockout length is a minimum amount of time in which the algorithm is paused.

111. The neuromodulation system of claim 103, wherein the electrical stimulation pulses are delivered at a pulse frequency of 1 Hz to 100 Hz.

112. The neuromodulation system of claim 103, wherein the electrical stimulation pulse generator is integrated into the earpiece.

113. A neuromodulation system comprising:
an electrical stimulation generator that delivers electrical stimulation to an earpiece that is configured to be placed inside an ear, the earpiece comprising one or more electrodes configured to stimulate one or more nerves that innervate at least a portion of the ear;
one or more sensors that measure data of a user's physiological state; and
a controller configured to receive and use the measured data to adjust one or more stimulation parameters of the electrical stimulation.

114. The neuromodulation system of claim 113, wherein one of the one or more sensors is a microphone that is worn in the ear to detect changes in the user's respiratory cycle.

115. The neuromodulation system of claim 113, wherein the earpiece comprises an infrared reflective light monitor configured to detect changes in the user's respiratory cycle.

116. The neuromodulation system of claim 113, wherein the electrical stimulation generator communicates wirelessly to a belt respiration sensor and delivers the electrical stimulation to the earpiece via a conduit.

117. The neuromodulation system of claim 113, wherein:
the one or more sensors comprises a sensor configured for detecting and measuring a quantitative value generally relating to a respiratory phase of the user, wherein the quantitative value is one or more of a respiration threshold, a sample check count, a respiration slope threshold, and a lockout length; and
the controller is configured to:
apply an algorithm to the quantitative value; and
determine the respiratory phase of the user based on an application of the algorithm to the quantitative value,
wherein the respiratory phase determined is an inhalation phase or an exhalation phase.

118. The neuromodulation system of claim 117, wherein:
the quantitative value is the respiration threshold, and wherein the respiration threshold is a minimum difference in amplitude between two sample values; or
the quantitative value is the sample check count, and wherein the sample check count is a minimum number of samples in a row required to be checked in order to consider whether the user has switched from one respiratory phase to another; or
the quantitative value is the respiration slope, and wherein the respiration slope threshold is a minimum slope value required to assign a change from one respiratory phase to another; or
the quantitative value is the lockout length, and wherein the lockout length is a minimum amount of time in which the algorithm is paused.

119. The neuromodulation system of claim 113, wherein the electrical stimulation is delivered at a pulse frequency of 1 Hz to 100 Hz.

120. The neuromodulation system of claim 113, wherein the electrical stimulation generator is integrated into the earpiece.

121. The neuromodulation system of claim 113, wherein one of the one or more sensors is configured to detect at least one of heart rhythm, heart rate or heart rate variability.

122. The neuromodulation system of claim 103, wherein one of one or more sensors is configured for measuring cytokine levels.
